(19) United States
(12) Patent Application Publication
(10) Pub. No.: US 2012/0035217 A1
(43) Pub. Date: Feb. 9, 2012

(54) Bis Aromatic Compounds for Use as LTC4 Synthase Inhibitors

(75) Inventors: Peter Nilsson, Uppsala (SE); Benjamin Pelcman, Uppsala (SE); Martins Katkevics, Riga (LV); Edgars Suna, Riga (LV); Ilya Popovs, Riga (LV)

(73) Assignee: BIOLIPOX AB, Uppsala (SW)

(21) Appl. No.: 13/256,041

(22) PCT Filed: Mar. 12, 2010
(86) PCT No.: PCT/GB10/00446

§ 371 Date: Oct. 20, 2011

Related U.S. Application Data
(60) Provisional application No. 61/202,547, filed on Mar. 12, 2009.

(51) Int. Cl. A61K 31/444; A61K 31/4184; C07D 249/04; A61K 31/4192; C07D 401/10; A61K 31/4439; C07D 401/14; C07D 213/74; A61K 31/44; A61P 11/00; A61P 29/00; A61P 11/08; A61P 11/02; A61P 27/02; A61P 17/00; A61P 9/00; A61P 1/00; A61P 13/00; A61P 25/00; A61P 5/00; A61P 43/00; A61P 31/04; A61P 31/10; A61P 31/12; A61P 7/06; A61P 35/00; A61P 11/06; A61P 19/02; C07D 235/06

(52) U.S. Cl. 514/333; 548/304.4; 514/394; 548/255; 514/359; 546/268.4; 514/340; 546/256; 546/275.4; 514/341; 546/272.7; 546/276.4; 514/343; 546/310; 514/352

(57) ABSTRACT

There is provided compounds of formula I,

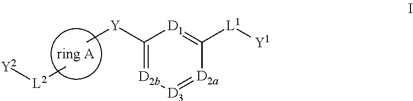

wherein Y, ring A, D₁, D₂ₐ, D_{2b}, D₃, L¹, Y¹, L² and Y² have meanings given in the description, and pharmaceutically-acceptable salts thereof, which compounds are useful in the treatment of diseases in which inhibition of leukotriene C₄ synthase is desired and/or required, and particularly in the treatment of a respiratory disorder and/or inflammation.

### FIELD OF THE INVENTION

This invention relates to novel pharmaceutically-useful compounds, which compounds are useful as inhibitors of the production of leukotrienes, such as leukotriene C₄. The compounds are of potential utility in the treatment of respiratory and/or inflammatory diseases. The invention also relates to the use of such compounds as medicaments, to pharmaceutical compositions containing them, and to synthetic routes for their production.

### BACKGROUND OF THE INVENTION

Arachidonic acid is a fatty acid that is essential in the body and is stored in cell membranes. They may be converted, e.g. in the event of inflammation, into mediators, some of which are known to have beneficial properties and others that are harmful. Such mediators include leukotrienes (formed by the action of 5-lipoxygenase (5-LO), which acts by catalysing the insertion of molecular oxygen into carbon position 5) and prostaglandins (which are formed by the action of cyclooxygenases (COXs)). Huge efforts have been devoted towards the development of drugs that inhibit the action of these metabolites as well as the biological processes that form them.

Of the leukotrienes, leukotriene (LT) B₄ is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. It has also been suggested that the CysLTs play a role in inflammatory mechanisms. The biological activities of the CysLTs are mediated through two receptors designated CysLT₁ and CysLT₂, but the existence of additional CysLT receptors has also been proposed. Leukotriene receptor antagonists (LTRas) have been developed for the treatment of asthma, but they are often highly selective for CysLT₁. It may be hypothesised that better control of asthma, and possibly also COPD, may be attained if the activity of both of the CysLT receptors could be reduced. This may be achieved by developing unselective LTRas, but also by inhibiting the activity of proteins, e.g. enzymes, involved in the synthesis of the CysLTs; 5-LO, 5-lipoxygenase-activating protein (FLAP), and leukotriene C₄ synthase may be mentioned. However, a 5-LO or a FLAP inhibitor would also decrease the formation of LTB₄. For a review on leukotrienes in asthma, see H.-E Claesson and S.-E. Dahlén J. Internal Med. 245, 205 (1999).

There are many diseases/disorders that are inflammatory in their nature or have an inflammatory component. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, LTRas have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomatology of the patients.

Thus, new and/or alternative treatments for respiratory and/or inflammatory disorders would be of benefit to all of the above-mentioned patient groups. In particular, there is a real and substantial unmet clinical need for an effective anti-inflammatory drug capable of treating inflammatory disorders, in particular asthma and COPD, with no real or perceived side effects.

The listing or discussion of an apparently prior-published document in this specification should not necessarily be taken as an acknowledgement that the document is part of the state of the art or is common general knowledge.

International patent application WO 2008/107661 discloses various biphenyl/diphenyl compounds that may be useful as LTC₄ synthase inhibitors, and of use therefore in the treatment of inflammation. However, the two phenyl rings are linked together with via a methylene group. Further, international patent application WO 2009/030887 discloses, for that same use, various biaryl compounds linked together with a carbonyl group (i.e. diarylketones). However, there is no specific disclosure in that application of a biaryl/diaryl compound in which one of the requisite aromatic rings is directly linked with a certain aromatic or vinylic group.

### DISCLOSURE OF THE INVENTION

According to the invention, there is provided a compound of formula I,

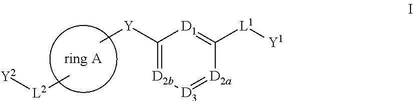

wherein
Y represents —C(O)— or —C(═N—OR²⁸)—;
R²⁸ represents hydrogen or C₁₋₆ alkyl optionally substituted by one or more fluoro atoms;
either one of D₂ₐ and D_{2b} represents D₂, and the other represents —C(T)═;
D₁, D₂ and D₃ respectively represent —C(R^{1a})═, —C(R^{1b})═ and —C(R^{1c})═, or, each of these may alternatively and independently represent —N═;
ring A represents:

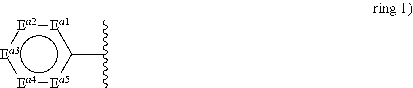

each of E^{a1}, E^{a2}, E^{a3}, E^{a4} and E^{a5} respectively represent —C(H)═, —C(R^{2b})═, —C(R^{2c})═, —C(R^{2d})═ and —C(H)═, or, each of E^{al}, E^{a2}, E^{a3}, E^{a4} and E^{a5} may alternatively and independently represent —N═;
one of R^{2b}, R^{2c} and R^{2d} represents the requisite -L²-Y² group, and the others independently represent hydrogen, -L^{1a}-Y^{1a} or a substituent selected from X¹;

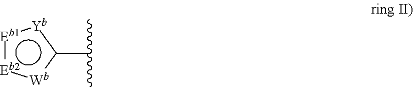

E^{b1} and E^{b2} respectively represent —C(R^{3a})═ and —C(R^{3b})═;
Y^{b} represents —C(R^{3c})═ or —N═;
W^{b} represents —N(R^{3d})—, —O— or —S—;
one of R^{3a}, R^{3b} and, if present, R^{3c} and R^{3d}, represents the requisite -L²-Y² group, and the remaining R^{3a}, R^{3b} and (if present) R^{3c} substituents represents hydrogen, -L^{1a}-Y^{1a} or a substituent selected from X², and the remaining R^{3d} substituent (if present) represents hydrogen or a substituent selected from R^{z1}; or

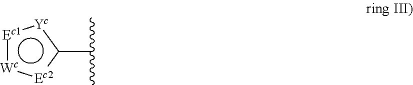

E^{c1} and E^{c2} each respectively represent —C(R^{4a})═ and —C(R^{4b})═;
Y^{c} represents —C(R^{4c})═ or —N═;
W′ represents —N(R^{4d})—, —O— or —S—;
one of R^{4a}, R^{4b} and, if present, R^{4c} and R^{4d} represents the requisite -L²-Y² group, and the remaining R^{4a}, R^{4b} and (if present) R^{4c} substituents represent hydrogen, -L^{1a}-Y^{1a} or a substituent selected from X³, and the remaining R^{4d} substituent (if present) represents hydrogen or a substituent selected from R^{z2};
R^{z1} and R^{z2} independently represent a group selected from Z^{1a};
R^{1a}, R^{1b}, R^{1c}, independently represent hydrogen, a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5b}, —OC(O)N(R^{6g})R^{7g}, —OS(O)₂R⁵ⁱ, —N(R^{5k})S(O)₂R^{5m}, —OC(O)R⁵ⁿ, —OC(O)OR⁵ or —OS(O)₂N(R⁶ⁱ)R⁷ⁱ;
X¹, X² and X³ independently represent a group selected from Z^{2a}, or, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5b}, —OC(O)N(R^{6g})R^{7g}, —OS(O)₂R⁵ⁱ, —N(R^{5k})S(O)₂R^{5m}, —OC(O)R⁵ⁿ, —OC(O)OR⁵ or —OS(O)₂N(R⁶ⁱ)R⁷ⁱ;
Z^{1a} and Z^{2a} independently represent —R^{5a}, —C(O)R^{5b}, —C(O)OR⁵⁰, —C(O)N(R^{6a})R^{7a}, —S(O)ₘR^{5j} or —S(O)₂N(R^{6h})R^{7h};
R^{5b} to R^{5h}, R^{5j}R^{5k}, R⁵ⁿ, R^{6a} to R⁶ⁱ, R^{7a}, R^{7b}, R^{7d} and R^{7f} to R⁷ⁱ independently represent, on each occasion when used herein, H or R^{5a}; or
any of the pairs R^{6a} and R^{7a}, R^{6b} and R^{7b}, R^{6d} and R^{7d}, R^{6f} and R^{7f}, R^{6g} and R^{7g}, R^{6h} and R^{7b} or R⁶ⁱ and R⁷ⁱ may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from F, Cl, ═O, —OR^{5h} and/or R^{5a};
R⁵ⁱ, R^{5m} and R^{5p} independently represent R^{5a};
R^{5a} represents, on each occasion when used herein, C₁₋₆ alkyl optionally substituted by one or more substituents selected from halo, —CN, —N₃, ═O, —OR^{8a}, —N(R^{8b})R^{8c}, —S(O)ₙR^{8d}, —S(O)₂N(R^{8e})R^{8f} and/or —OS(O)₂N(R^{8g})R^{8h};
n represents 0, 1 or 2;
R^{8a}, R^{8b}, R^{8d}, R^{8e} and R^{8g} independently represent H or C₁₋₆ alkyl optionally substituted by one or more substituents selected from halo, ═O, —OR^{11a}, —N(R^{12a})R^{12b} and/or —S(O)₂-M¹;
R^{8c}, R^{8f} and R^{8h} independently represent H, —S(O)₂CH₃, —S(O)₂CF₃ or C₁₋₆ alkyl optionally substituted by one or more substituents selected from F, Cl, ═O, —OR^{13a}, —N(R^{14a})R^{14b} and/or —S(O)₂-M²; or
R^{8b} and R^{8c}, R^{8e} and R^{8f} or R^{8g} and R^{8h} may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from F, Cl, ═O and/or C₁₋₃ alkyl optionally substituted by one or more substituents selected from ═O and fluoro;
M¹ and M² independently represent N(R^{15a})R^{15b} or C₁₋₃ alkyl optionally substituted by one or more fluoro atoms;
R^{11a} and R^{13a} independently represent H or C₁₋₃ alkyl optionally substituted by one or more fluoro atoms;
R^{12a}, R^{12b}, R^{14a}, R^{14b}, R^{15a} and R^{15b} independently represent H, —CH₃ or —CH₂CH₃,
T represents T¹, T², T³, T⁴, T⁵, T⁶ or T⁷;
T¹ is an aromatic ring represented by the following substructure:

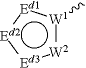

in which:
(i) W¹ represents —C═;
W² represents —C(R¹)═ or —C(Y³)═; and
one of E^{d1}, E^{d2} or E^{d3} represents —N(R²)—, —N(Y⁴)—, —O— or —S—, and the other two independently represent —C(R¹)═, —C(Y³)═ or —N═,
wherein there is one Y³ or Y⁴ group present;
   (ii) W¹ represents —C═;
W² represents —N(Y⁴)—;
E^{d1}, E^{d2} and E^{d3} independently represent —C(R¹)═ or —N═; or
   iii) W¹ represents —N—;
W² represents —C(R¹)═ or —C(Y³)═;
E^{d1}, E^{d2} and E^{d3} independently represent —C(R¹)═, —C(Y³)═ or —N═,
wherein there is one Y³ group present;
   T² is an aromatic ring represented by the following substructure:

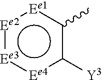

in which:
one of E^{e1}, E^{e2} E^{e3} or E^{e4} represents —C(R¹)═ and the others independently represent —C(R¹)═ or —N═;
T³ is an aromatic bicyclic ring represented by the following substructure:

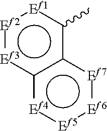

any three of E^{f1} to E^{f7} represent —C(R¹)═, and the others represent —C(R¹)═ or —N═;
T⁴ is an aromatic bicyclic ring represented by the following substructure:

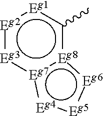

E^{g1}, E^{g2}, E^{g3} and E^{g5} independently represent —C(R¹)═ or —N═;
one of E^{g4} and E^{g6} represents —N(R²)—, —O— or —S— and the other represents —C(R¹)═ or —N═;
E^{g7} and E^{g8} both represent a carbon atom, or one of E^{g7} and E^{g8} represents a carbon atom and the other represents a nitrogen atom,
but wherein in the bicyclic ring, no more than four nitrogen atoms are present (i.e. no more than four of E^{g1} to E^{g8} may represent a nitrogen atom represented by —N═ or —N(R²)—);
T⁵ is an aromatic bicyclic ring represented by the following substructure:

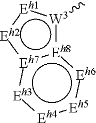

E^{h3}, E^{h4}, E^{h5} and E^{h6} independently represent —C(R¹)═ or —N═;
(i) W³ represents —C═;
E^{h1} represents —C(R¹)═ or —N═;
E^{h2} represents —N(R²)—, —O— or —S—, or, provided that E^{h7} or E^{h8} is a nitrogen atom, —C(R¹)═;
E^{h7} and E^{h8} both represent a carbon atom, or one of E^{h7} and E^{h8} represents a carbon atom and the other represents a nitrogen atom;
   (ii) W³ represents —N—;
E^{h1} and E^{h2} independently represent —C(R¹)═ or —N═;
E^{h7} and E^{h8} both represent a carbon atom,
   but wherein in the bicyclic ring, no more than four nitrogen atoms are present (i.e. no more than four of W³, and E^{h1} to E^{h8} may represent a nitrogen atom represented by —N═ or —N(R²)—);
   T⁶ is a ring represented by the following substructure:

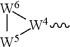

W⁴ represents —C(R³)— or —N—;
when W⁴ represents —C(R³)—, then W⁵ represents —C(R³)(Y⁵)— or —N(Y⁶)—;
when W⁴ represents —N—, then W⁵ represents —C(R³)(Y⁵)—;
W⁶ represents an C₁₋₅ alkylene or C₁₋₅ heteroalkylene chain both of which are optionally substituted by one or more substituents selected from G^{x};
T⁷ is a cyclic or acyclic alkene represented by the following substructure:

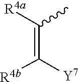

R^{4a} and R^{4b} independently represent G^{w}, or R^{4a} and R^{4a} are connected together to form, along with the two alkene carbons to which they are necessarily attached, a C₃₋₇ cycloalkylene or 3- to 8-membered heterocycloalkylene ring both optionally substituted by one or more substituents selected from G^{x};
each R¹ represents, on each occasion when used herein, hydrogen, halo, —R^{25a}, —C(O)R^{25b}, —CN, —C(O)N(R^{26a})R^{27a}, —N(R^{26b})R^{27b}, —N(R^{25c})C(O)R^{26c}, —N(R^{25d})C(O)OR^{26d}, —OR^{25e}, —OS(O)₂R^{25f}, —S(O)ₘ₁R^{25g}, —OC(O)R^{25h} or —S(O)₂N(R^{26e})R^{27e};
each R² represents, on each occasion when used herein, hydrogen, —R^{25a}, —C(O)R^{25b} or —C(O)N(R^{26a})R^{27a};
each R³ represents, on each occasion when used herein, hydrogen, —R^{35a}, —CN, —N(R^{36b})R^{37b} or —OR^{35d};
G^{w} represents hydrogen, halo, —R^{45a}, —C(O)R^{45b}, —CN, —C(O)N(R^{46a})R^{47a}, —N(R^{46b})R^{47b}, —N(R^{46c})C(O)R^{46c}, —N(R^{45d})C(O)OR^{46d}, —OR^{45e}, —OS(O)₂R^{45f}, —S(O)ₘ₁R^{45g}, —OC(O)R^{45h} or —S(O)₂N(R^{46e})R^{47e};
G^{x} represents F, —R^{55a}, —C(O)R^{55b}, —CN, —C(O)N(R^{56a})R^{57a}, —N(R^{56b})R^{57b}, —N(R^{55c})C(O)R^{56c}, —N(R^{55d})C(O)OR^{56d}, —OR^{55e}, —OS(O)₂R^{55f}, —S(O)ₘ₁R^{55g}, —OC(O)R^{55h}, —S(O)₂N(R^{56e})R^{57e} or ═O;
m1 represents, on each occasion when used herein, 0, 1 or 2;
R^{25b} to R^{25e}, R^{25g}, R^{25h}, R^{26a} to R^{26e}, R^{27a}, R^{27b}, R^{27e}, independently represent, on each occasion when used herein, H or R^{25a}; or
any of the pairs R^{26a} and R^{27a}, R^{26b} and R^{27b}, R^{26e} and R^{27e}, may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from fluoro, ═O, —O—C₁₋₄ alkyl and/or C₁₋₄ alkyl;
R^{25f} represents R^{25a};
R^{36b}, R^{37b} and R^{35d} independently represent hydrogen or R^{35a}; or
R^{36b} and R^{37b} may be linked together to form a 3- to 6-membered ring optionally containing one further heteroatom, and which ring is optionally substituted by one or more substituents selected from F and methyl;
R^{45b}, R^{46a}, R^{47a}, R^{46c}, R^{46d}, R^{45h}, R^{46e} and R^{47e} independently represent hydrogen or R^{45a};
R^{45g}, R^{46b}, R^{47b}, R^{45c}, R^{45d}, R^{45e} and R^{45f} independently represent R^{45a}; or
any of the pairs R^{46a} and R^{47a}, R^{46b} and R^{47b}, and R^{46e} and R^{47e} may be linked together to form a 3- to 6-membered ring optionally containing one further heteroatom, and which ring is optionally substituted by one or more substituents selected from F and methyl;
R^{55b} to R^{55e}, R^{55g}, R^{55h}, R^{56a} to R^{56e}, R^{57a}, R^{57b}, R^{57e}, independently represent, on each occasion when used herein, H or R^{55a}; or
any of the pairs R^{56a} and R^{57a}, R^{56b} and R^{57b}, R^{56e} and R^{57e}, may be linked together to form a 3- to 6-membered ring optionally containing one further heteroatom, and which ring is optionally substituted by one or more substituents selected from F and methyl;
R^{25a} represents, on each occasion when used herein, C₁₋₆ alkyl optionally substituted by one or more substituents selected from fluoro, —CN, ═O and —O—C₁₋₄ alkyl;
R^{35a}, R^{45a} and R^{55a} independently represent C₁₋₄ alkyl optionally substituted by one or more fluoro atoms;
Y², Y³, Y⁴, Y⁵, Y⁶ and Y⁷ independently represent, on each occasion when used herein:
(a) an aryl group or a heteroaryl group (both of which groups are optionally substituted by one or more substituents selected from A); or
(b) C₁₋₁₂ alkyl or a heterocycloalkyl group, both of which are optionally substituted by one or more substituents selected from G¹ and/or Z¹,
but wherein at least one Y², Y³, Y⁴, Y⁵, Y⁶, Y⁷ group is present that represents an aryl group or a heteroaryl group (both of which groups are optionally substituted by one or more substituents selected from A);
Y¹ and Y^{1a} independently represent —C(O)OR^{9a} or 5-tetrazolyl;
R^{9a} represents:
(i) hydrogen; or
(ii) C₁₋₈ alkyl or a heterocycloalkyl group, both of which are optionally substituted by one or more substituents selected from G¹ and/or Z¹;
A represents, on each occasion when used herein:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents, on each occasion when used herein, halo, cyano, —N₃, —NO₂, —ONO₂ or -A¹-R^{16a};
wherein A¹ represents a single bond or a spacer group selected from —C(O)A²-, —S—, —S(O)ₘₓA³-, —N(R^{17a})A⁴- or —OA⁵-, in which:
A² represents a single bond, —O—, —N(R^{17b})— or —C(O)—;
A³ represents a single bond, —O— or —N(R^{17c})—;
A⁴ and A⁵ independently represent a single bond, —C(O)—, —C(O)N(R^{17d})—, —C(O)O—, —S(O)₂— or —S(O)₂N(R^{17e})—;
Z¹ represents, on each occasion when used herein, ═O, ═S, ═NOR^{16b}, ═NS(O)₂N(R^{17f})R^{16c}, ═NCN or ═C(H)NO₂;
B represents, on each occasion when used herein:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents, on each occasion when used herein, halo, cyano, —N₃, —NO₂, —ONO₂ or -A⁶-R^{18a};
wherein A⁶ represents a single bond or a spacer group selected from —C(O)A⁷-, —S—, —S(O)ₘₓA⁹-, —N(R^{19a})A⁹- or —OA¹⁰-, in which:
A⁷ represents a single bond, —O—, —N(R^{19b})— or —C(O)—;
A⁸ represents a single bond, —O— or —N(R^{19c})—;
A⁹ and A¹⁰ independently represent a single bond, —C(O)—, —C(O)N(R^{19d})—, —C(O)O—, —S(O)₂— or —S(O)₂N(R^{19e})—;
Z² represents, on each occasion when used herein, ═O, ═S, ═NOR^{19b}, ═NS(O)₂N(R^{19f})R^{18c}, ═NCN or ═C(H)NO₂;
R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by one or more substituents selected from G³ and/or Z³; or
any pair of R^{16a} to R^{16c} and R^{17a} to R^{17f}, and/or R^{18a} to R^{18c} and R^{18a} to R^{18f}, may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents, on each occasion when used herein, halo, cyano, —N₃, —NO₂, —ONO₂ or -A¹¹-R^{20a};
wherein A¹¹ represents a single bond or a spacer group selected from —C(O)A¹²-, —S—, —S(O)ₘₓA¹³-, —N(R^{21a})A¹⁴- or —OA¹⁵-, in which:
A¹² represents a single bond, —O—, —N(R^{21b})— or —C(O)—;
A¹³ represents a single bond, —O— or —N(R^{21c})—;
A¹⁴ and A¹⁵ independently represent a single bond, —C(O)—, —C(O)N(R^{21d})—, —C(O)O—, —S(O)₂— or —S(O)₂N(R^{21e})—;
Z³ represents, on each occasion when used herein, ═O, ═S, ═NOR^{2m}, ═NS(O)₂N(R^{21f})R^{20c}, ═NCN or ═C(H)NO₂;
R^{20a}, R^{20b}, R^{20c}, R^{21a}, R^{21b}, R^{21c}, R^{21d}, R^{21e} and R^{21f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{22a})R^{23a}, —OR^{22b} and ═O; and
iii) an aryl or heteroaryl group, both of which are optionally substituted by one or more substituents selected from halo, C₁₋₄ alkyl (optionally substituted by one or more substituents selected from ═O, fluoro and chloro), —N(R^{22c})R^{23b} and —OR^{22d}; or
any pair of R^{20a} to R^{20c} and R^{21a} to R^{21f} may, for example when present on the same or on adjacent atoms, be linked together to form with those, or other relevant, atoms a further 3- to 8-membered ring, optionally containing 1 to 3 heteroatoms and/or 1 or 2 double bonds, which ring is optionally substituted by one or more substituents selected from halo, C₁₋₄ alkyl, —N(R^{22e})R^{23c}, —OR^{22f} and ═O;
L¹ and L^{1a} independently represent a single bond or —(CH₂)ₚ-Q-(CH₂)_{q}—;
Q represents —C(R^{y1})(R^{y2})—, —C(O)—, —N(R^{y3})— or —O—;
p and q independently represent 0, 1 or 2, but wherein the sum of p and q does not exceed 2;
L² represents a single bond or a spacer group selected from —C(R^{y4})(R^{y5})—, —N(R^{17a})-A¹⁶-, and —OA¹⁷-;
A¹⁶ represents a direct (i.e. a single) bond, —C(O)—, —C(O)N(R^{17b})—, —C(O)C(R^{y6})(R^{y7})— or —S(O)₂—;
A¹⁷ represents a direct bond or —C(R^{y8})(R^{y9})—;
R^{y1}, R^{y2}, R^{y4}, R^{y5}, R^{y6}, R^{y7}, R^{y8} and R^{y9} independently represent H, fluoro or C₁₋₃ alkyl optionally substituted by one or more fluoro atoms; or
R^{y1} and R^{y2}, R^{y4} and R^{y5}, R^{y6} and R^{y7} and R^{y8} and R^{y9} may be linked together to form a 3- to 6-membered ring optionally substituted by one or more substituents selected from fluoro and C₁₋₂ alkyl;
R^{y3} represents hydrogen or C₁₋₃ alkyl;
R^{17a} and R^{17b} independently represent hydrogen, C₁₋₆ alkyl (optionally substituted by one or more substituents selected from fluoro, —CN, —OH, —OCH₃, —OCH₂CH₃ and/or ═O), aryl or heteroaryl (both of which latter two groups are optionally substituted by one or more substituents selected from halo, —R^{18a}, —C(O)R^{18b}, —CN, —C(O)N(R^{18c})R^{18d}, —N(R^{18e})R^{18f}, —N)R^{18g})C(O)R^{18h}, —N(R¹⁸ⁱ)C(O)OR^{18j}, —OR^{18k}, —OS(O)₂R^{18m}, —S(O)ₘR¹⁸ⁿ, —OC(O)R^{18p} or —S(O)₂N(R^{18q})R^{18r});
m represents, on each occasion when used herein, 0, 1 or 2;
mx represents, on each occasion when used herein, 1 or 2;
R^{18a}, R^{18b}, R^{18c}, R^{18d}, R^{18e}, R^{18f}, R^{18g}, R^{18h}, R¹⁸ⁱ, R^{18j}, R^{18k}, R¹⁸ⁿ, R^{18p}, R^{18q} and R^{18r} independently represent hydrogen or C₁₋₃ alkyl optionally substituted by one or more fluoro atoms;
R^{18m} represents C₁₋₃ alkyl optionally substituted by one or more fluoro atoms;
or a pharmaceutically-acceptable salt thereof,
which compounds and salts are referred to hereinafter as “the compounds of the invention”. Such compounds are characterised in that there is present a —C(T)=moiety, in which T is a specific aromatic moiety, a specific 3-membered ring or a specific alkene as defined herein.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl, or, C_{1-q} alkylene, groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group, or, if there is an alkene group present, a C_{3-q} cycloalkenyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group). Further, heteroalkylene groups may be mentioned, by which we mean C_{1-q} alkylene groups, but in which at least one of the carbon atoms is replaced with a heteroatom (e.g. nitrogen, oxygen or sulfur). Where the number of carbon atoms permits, C_{1-q} alkyl groups may also be spiro-groups (i.e. two cycloalkyl rings linked together by a single common carbon atom), although they are preferably not so.

The term “halo”, when used herein, includes fluoro, chloro, bromo and iodo.

Heterocycloalkyl groups that may be mentioned include non-aromatic monocyclic and bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten). Further, such heterocycloalkyl groups may be saturated or unsaturated containing one or more double and/or triple bonds, forming for example a C_{2-q} (e.g. C_{4-q}) heterocycloalkenyl (where q is the upper limit of the range) or a C_{7-q} heterocycloalkynyl group. C_{2-q} heterocycloalkyl groups that may be mentioned include 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form.

For the avoidance of doubt, the term “bicyclic” (e.g. when employed in the context of heterocycloalkyl groups) refers to groups in which the second ring of a two-ring system is formed between two adjacent atoms of the first ring. The term “bridged” (e.g. when employed in the context of heterocycloalkyl groups) refers to monocyclic or bicyclic groups in which two non-adjacent atoms are linked by either an alkylene or heteroalkylene chain (as appropriate).

Aryl groups that may be mentioned include C₆₋₁₄ (such as C₆₋₁₃ (e.g. C₆₋₁₀)) aryl groups. Such groups may be monocyclic or bicyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are preferably linked to the rest of the molecule via an aromatic ring.

Heteroaryl groups that may be mentioned include those which have between 5 and 14 (e.g. 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heteroaryl groups that may be mentioned include oxazolopyridyl (including oxazolo[4,5-b]pyridyl, oxazolo[5,4-b]pyridyl and, in particular, oxazolo[4,5-c]pyridyl and oxazolo[5,4-c]pyridyl), thiazolopyridyl (including thiazolo[4,5-b]pyridyl, thiazolo[5,4-b]pyridyl and, in particular, thiazolo[4,5-c]pyridyl and thiazolo[5,4-c]pyridyl) and, more preferably, benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselena-diazolyl (including 2,1,3-benzoselenadiazolyl), benzothienyl, carbazolyl, chromanyl, cinnolinyl, furanyl (i.e. furyl), imidazolyl, imidazopyridyl (such as imidazo[4,5-b]pyridyl, imidazo[5,4-b]pyridyl and, preferably, imidazo[1,2-a]pyridyl), indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. However, when heteroaryl groups are polycyclic, they are preferably linked to the rest of the molecule via an aromatic ring. Heteroaryl groups may also be in the N- or S-oxidised form.

Heteroatoms that may be mentioned include phosphorus, silicon, boron, tellurium, selenium and, preferably, oxygen, nitrogen and sulphur.

For the avoidance of doubt, in cases in which the identity of two or more substituents in a compound of the invention may be the same, the actual identities of the respective substituents are not in any way interdependent. For example, in the situation in which X¹ and X² both represent R^{5a}, i.e. a C₁₋₆ alkyl group optionally substituted as hereinbefore defined, the alkyl groups in question may be the same or different. Similarly, when groups are substituted by more than one substituent as defined herein, the identities of those individual substituents are not to be regarded as being interdependent. For example, when there are two X¹ substituents present, which represent —R^{5a} and —C(O)R^{5b} in which R^{5b} represents R^{5a}, then the identities of the two R^{5a} groups are not to be regarded as being interdependent. Likewise, when Y² or Y³ represent e.g. an aryl group substituted by G¹ in addition to, for example, C₁₋₈ alkyl, which latter group is substituted by G¹, the identities of the two G¹ groups are not to be regarded as being interdependent.

For the avoidance of doubt, when a term such as “R^{5a} to R^{5h}” is employed herein, this will be understood by the skilled person to mean R^{5a}, R^{5b}, R^{5c}, R^{5d}, R^{5e}, R^{5f}, R^{5g} and R^{5h} inclusively.

For the avoidance of doubt, when the term “an R⁵ group” is referred to herein, we mean any one of R^{5a} to R^{5k}, R^{5m}, R⁵ⁿ or R^{5p}.

For the avoidance of doubt, where it is stated herein that “any pair of R^{16a} to R^{16c} and R^{17a} to R^{17f} . . . may . . . be linked together”, we mean that any one of R^{16a}, R^{16b} or R^{16c} may be linked with any one of R^{17a}, R^{17b}, R^{17c}, R^{17d}, R^{17e} or R^{17f} to form a ring as hereinbefore defined. For example, R^{16a} and R^{17b} (i.e. when a G¹ group is present in which G¹ represents -A¹-R^{16a}, A¹ represents —C(O)A² and A² represents —N(R^{17b})—) or R^{16c} and R^{17f} may be linked together with the nitrogen atom to which they are necessarily attached to form a ring as hereinbefore defined.

The skilled person will appreciate that, given that there is an essential ‘-L²-Y²’ group present in the compound of formula I, then when, for example, ring A represents ring I), then at least one of —C(R^{2b})═, —C(R^{2c})═ and —C(R^{2d})═ must be present, in which the any one of the relevant R^{2b}, R^{2c} and R^{2d} groups represents the essential -L²-Y² group.

For the avoidance of doubt, the following compounds of formula I are included within the scope of the invention:

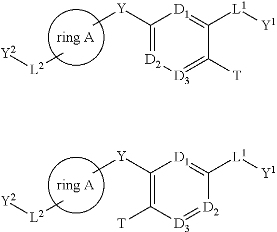

wherein the integers are as hereinbefore defined.

Compounds of the invention that may be mentioned include those in which, for example, when D₂ₐ represents T, then:

the D₁ to D₃-containing ring does not contain —N═ (i.e. D₁, D₂ and D₃ respectively represent —C(R^{1a})═, —C(R^{1b})═ and —C(R^{1c})═);
ring A (e.g. when it represents ring (I)) does not contain —N═ (e.g. E^{a1}, E^{a2}, E^{a3}, E^{a4} and E^{a5} respectively represent —C(H)═, —C(R^{2b})═, —C(R^{2c})═, —C(R^{2d})═ and —C(H)═);
Z^{1a} and Z^{2a} do not represent —C(O)OR^{5c} (i.e. each independently represent —R^{5a}, —C(O)R^{5b}, —C(O)N(R^{6a})R^{7a}, —S(O)ₘR^{5j} or —S(O)₂N(R^{6h})R^{7h}), especially when D₃ represents —C(R^{1c})═ and R^{1c} represents Z^{2a}.

Further compounds of the invention that may be mentioned include those in which:

when D₃ represents —C(R^{1c})═, then R^{1c} preferably represents hydrogen or a substituent selected from R^{5a} and, preferably, halo and —CN (most preferably, R^{1c} represents hydrogen);
when D₁ and D₂ respectively represent —C(R^{1a})═ and —C(R^{1b})═, then R^{1a} and R^{1b} preferably (and independently) represent hydrogen or a substituent selected from R^{5a} and, preferably, halo and —CN (most preferably, they represent hydrogen);
one of R^{2b}, R^{2c} and R^{2d} (preferably R^{2c}) represents the requistite -L²-Y² group and the others (e.g. R^{2b} and R^{2d}) independently represent hydrogen or a substituent selected from R^{5a} and, preferably, halo and —CN (most preferably, they represent hydrogen).

Compounds of the invention that may be mentioned include those in which T represents any one of T¹, T², T³, T⁴, T⁵, T⁶ or T⁷. Further compounds that may be mentioned include those in which T represents any two or more of T¹, T², T³, T⁴, T⁵, T⁶ and T⁷.

Preferred compounds of the invention include those in which:

Y represents —C(O)— or —C(═N—OR²⁸)— (preferably —C(O)—);
D₂ₐ represents D₂, and D_{2b} represents T;
when T represents T², then two, preferably, one, or more preferably, none of E^{e1}, E^{e3} or E^{e4} represents —N═;
when T represents T³ then, preferably, the total number of nitrogen atoms is preferably less than 4, more preferably less than 3 (especially less than 2, particularly 1 and more particularly there are no nitrogen atoms);
when T represents T³ then, preferably, each ring has 2, preferably 1 or more preferably no nitrogen atoms;
when T represents T⁴ then, the total number of nitrogen atoms is preferably less than 4, more preferably less than 3 (especially less than 2, particularly 1 and more particularly there are no nitrogen atoms);
when T represents T⁵ then, preferably, each ring has 2, preferably 1 or more preferably no nitrogen atoms;
the pairs R^{26a} and R^{27e}, R^{26b} and R^{27b}, R^{26e} and R^{27e}, when linked form a 5- to 6-membered ring, which ring optionally contains a further heteroatom (such as nitrogen or oxygen) in addition to the nitrogen atom to which these substituents are necessarily attached, and which ring is optionally substituted by one or more substituents selected from fluoro and methyl;
the pairs R^{26a} and R^{27e}, R^{26b} and R^{27b}, R^{26e} and R^{27e}, are preferably not linked together;
R^{36b} and R^{37b} when linked together form a 5- to 6-membered ring optionally containing one further heteroatom (e.g. nitrogen or oxygen), and which ring is optionally substituted by one or more substituents selected from F and methyl;
R^{36b} and R^{37b} are preferably not linked together;
the pairs R^{36b} and R^{37b}, R^{46e} and R^{47e}, R^{46b} and R^{47b}, R^{46e} and R^{47e}, R^{56e} and R^{57e}, R^{56b} and R^{57b}, and R^{56e} and R^{57e}, when linked together, form a 5- to 6-membered ring optionally containing one further heteroatom (e.g. nitrogen or oxygen), and which ring is optionally substituted by one or more substituents selected from F and methyl;
the pairs R^{36b} and R^{37b}, R^{46e} and R^{47e}, R^{46b} and R^{47b}, R^{46e} and R^{47e}, R^{56e} and R^{57e}, R^{56b} and R^{57b}, and R^{56e} and R^{57e}, are preferably not linked together;
R^{25a} represents, on each occasion when used herein, C₁₋₄ alkyl optionally substituted by one or more substituents selected from fluoro and ═O;
R^{35a}, R^{45a} and R^{55a} independently represent C₁₋₄ (e.g. C₁₋₂) alkyl (e.g. methyl) optionally substituted by one or more fluoro atoms (so forming for example a trifluoromethyl group).

Compounds of the invention that may be mentioned include those in which:

when R^{5a} represents C₁₋₆ alkyl, then that alkyl group may not be substituted at a terminal position of the alkyl group by both ═O and —OR^{8a} (hence, when R^{5a} represents C₁₋₆ alkyl, then it may not be substituted by a —C(O)OR^{8a} group);
when R^{5a} represents C₁₋₆ alkyl, then that alkyl group may not be substituted at a terminal position of the alkyl group by both ═O and —N(R^{8b})R^{8c} (hence, when R^{5a} represents C₁₋₆ alkyl, then it may not be substituted by a —C(O)N(R^{8b})R^{8c} group);
when any of R^{8a}, R^{8b}, R^{8d} and R^{8e} represent C₁₋₆ alkyl, then that alkyl group may not be substituted at a terminal position of the alkyl group by both ═O and —OR^{11a} (hence, when such groups represent C₁₋₆ alkyl, then it may not be substituted by a —C(O)OR^{11a} group);
when any of R^{8a}, R^{8b}, R^{8d} and R^{8e} represent C₁₋₆ alkyl, then that alkyl group may not be substituted at a terminal position of the alkyl group by both ═O and —N(R^{12a})R^{12b} (hence, when such groups represent C₁₋₆ alkyl, then it may not be substituted by a —C(O)N(R^{12a})R^{12b} group);
when any of R^{8c} and/or R^{8f} represent C₁₋₃ alkyl, then that alkyl group may not be substituted at a terminal position of the alkyl group by both ═O and —OR^{13a} (hence, when such groups represent C₁₋₃ alkyl, then it may not be substituted by a —C(O)OR^{13a} group);
when any of R^{8c} and/or R^{8f} represent C₁₋₃ alkyl, then that alkyl group may not be substituted at a terminal position of the alkyl group by both ═O and —N(R^{14a})R^{14b} (hence, when such groups represent C₁₋₃ alkyl, then it may not be substituted by a —C(O)N(R^{14a})R^{14b} group);
when R^{25a} represents a C₁₋₆ alkyl group, then that alkyl group may not be substituted at a terminal position by both a ═O and a —OC₁₋₄ alkyl group, i.e. it may not be substituted by a —COOC₁₋₄ alkyl;
M¹ and M² independently represent —CH₂CH₃, or, preferably, —CH₃, —CF₃ or —N(R^{15a})R^{15b};
R^{11a} and R^{13a} independently represent —CHF₂ or, preferably H, —CH₃, —CH₂CH₃ or —CF₃.

Preferred compounds of the invention include those in which:

when there is a Y³, Y⁴, Y⁵, Y⁶ or Y⁷ group present, then it preferably represents a cyclic group optionally substituted as herein defined (e.g. a cycloalkyl or heterocycloalkyl group (both of which are optionally substituted by one or more substituents selected from G¹ and/or Z¹) or, preferably, aryl or heteroaryl (both of which are optionally substituted by one or more substituents selected from A));
Y² preferably represents a cyclic group optionally substituted as herein defined (e.g. a cycloalkyl or heterocycloalkyl group (both of which are optionally substituted by one or more substituents selected from G¹ and/or Z¹) or, preferably, aryl or heteroaryl (both of which are optionally substituted by one or more substituents selected from A));
both Y² and, if present, Y³ to Y⁷ represent aryl or heteroaryl (both of which are optionally substituted by one or more substituents selected from A).

Preferred compounds of the invention include those in which:

one (e.g. D₁ or D₃) or none of D₁, D₂ and D₃ represent —N═;
D₁, D₂ and D₃ respectively represent —C(R^{1a})═, —C(R^{1b})═ and —C(R^{1c})═;
R^{1a} and R^{1c} independently represent hydrogen;
when ring A represents ring (I), then two, preferably, one or, more preferably, none of E^{a1}, E^{a2}, E^{a3}, E^{a4} and E^{a5} represent —N═;
E^{a1}, E^{a2}, E^{a3}, E^{a4} and E^{a5} respectively represent —C(H)═, —C(R^{2b})═, —C(R^{2c})═, —C(R^{2d})═ and —C(H)═;
R² represents the requisite -L²-Y² group;
only one of R^{2b}, R^{2c} and R^{2d} (e.g. R^{2b}) may represent -L^{1a}-Y^{1a};
one of R^{2b} and R^{2d} (e.g. R^{2b}) represents hydrogen or -L^{1a}-Y^{1a} and the other represents hydrogen or a substituent selected from X¹;
when one of R^{2b}, R^{2c} and R^{2d} represents -L^{1a}-Y^{1a}, then it is preferably tetrazolyl or, more preferably, —COOR^{9a}, in which R^{9a} is preferably H;
R³ and R^{3d} independently represent unsubstituted C₁₋₆ (e.g. C₁₋₃) alkyl, or, preferably, hydrogen;
for example when ring A represents ring (II) then, one of R^{3a} and R^{3b} represents a substituent X² or, more preferably, H or -L^{1a}-Y^{1a}, and the other represents the requisite -L²-Y² group;
R^{4b} and R^{4c} independently represent unsubstituted C₁₋₆ (e.g. C₁₋₃) alkyl, or, preferably, hydrogen;
for example when ring A represents ring (III) then, one of R^{4a} and, if present, R^{4d} represents a substituent X³ or, more preferably, H or -L^{1a}-Y^{1a}, and the other represents the requisite -L²-Y² group;
when any one of R^{3a}, R^{3b}, R^{3c}, R^{3d}, R^{4a}, R^{4b}, R^{4c} or R^{4d} (e.g. R^{3a}, R^{3b}, R^{3a} or R^{4d}), represents -L^{1a}-Y^{1a}, then it is preferably a 5-tetrazolyl group or —COOR^{9a}, in which R^{9a} is preferably H;
X¹, X² and X³ independently represent halo (e.g. chloro or fluoro), —R^{5a}, —CN and —OR^{5h};
Z^{1a} and Z^{2a} independently represent —R^{5a};
when any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ are linked together, they form a 5- or 6-membered ring optionally substituted by F, —OCH₃ or, preferably, ═O or R^{5a}, and which ring optionally contains an oxygen or nitrogen heteroatom (which nitrogen heteroatom may be optionally substituted, for example with a methyl group, so forming e.g. —N(H)— or —N(CH₃)—);
R^{5c}, R^{5j} and R^{6e} independently represent R^{5a};
when R^{5a}, R^{8a}, R^{8b}, R^{8d}, R^{8e} and R^{8g} represent C₁₋₆ alkyl optionally substituted by one or more halo substituents, then those halo substituents are preferably Cl or, more preferably, F;
R^{5a} represents C₁₋₆ (e.g. C₁₋₄) alkyl optionally substituted by one or more substituents selected from Cl, ═O, —N(R^{8b})R^{8c} and, preferably, F and —OR^{8a};
m and n independently represent 2;
when any one of R^{8a}, R^{8b}, R^{8d}, R^{8e} and R^{8g} represents C₁₋₆ alkyl substituted by halo, then preferred halo groups are chloro and, preferably, fluoro;
R^{8a}, R^{8b}, R^{8d}, R^{8e} and R^{8g} independently represent H or C₁₋₃ alkyl optionally substituted by one or more fluoro atoms;
R^{8c}, R^{8f} and R^{8h} independently represent H, —S(O)₂CH₃, —S(O)₂CF₃ or C₁₋₃ alkyl optionally substituted by one or more fluoro atoms, or the relevant pairs (i.e. R^{8b} and R^{8c}, R^{8e} and R^{8f} or R^{8g} and R^{8h}) are linked together as defined herein;
when R^{8b} and R^{8c}, R^{8e} and R^{8f} or R^{8g} and R^{8h} are linked together, they form a 5- or 6-membered ring, optionally substituted by F, ═O or —CH₃;
M¹ and M² independently represent —CH₃ or —CF₃;
R^{11a}, R^{12a}, R^{12b}, R^{13a}, R^{14a}, R^{14b}, R^{15a} and R^{15b} independently represent H or —CH₃;
R^{9a} represents hydrogen or C₁₋₄ (e.g. C₁₋₃) alkyl optionally substituted by one or more halo (e.g. fluoro) atoms;
A represents aryl (e.g. phenyl) optionally substituted by B; C₁₋₆ alkyl optionally substituted by G¹ and/or Z¹; or G¹;
G¹ represents halo, cyano, or -A¹-R^{16a};
A¹ represents —C(O)A², —N(R^{17a})A⁴- or —OA⁵-;
A² represents a single bond or —O—;
A⁴ represents —C(O)N(R^{17d})—, —C(O)O— or, more preferably, a single bond or —C(O)—;
A⁶ represents —C(O)— or, preferably, a single bond;
Z¹ represents ═NCN, preferably, ═NOR^{16b} or, more preferably, ═O;
B represents heteroaryl (e.g. oxazolyl, thiazolyl, thienyl or, preferably, pyridyl) or, more preferably, aryl (e.g. phenyl) optionally substituted by G²; C₁₋₆ alkyl optionally substituted by G² and/or Z²; or, preferably G²,
G² represents cyano or, more preferably, halo or -A⁶-R^{18a};
A⁶ represents a single bond, —N(R^{19a})A⁹- or —OA¹⁰-;
A⁹ represents —C(O)N(R^{19d})—, —C(O)O— or, more preferably, a single bond or —C(O)—;
A¹⁹ represents a single bond;
Z² represents ═NCN, preferably, ═NOR^{18b} or, more preferably, ═O;
R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are independently selected from hydrogen, aryl (e.g. phenyl) or heteroaryl (which latter two groups are optionally substituted by G³) or C₁₋₆ (e.g. C₁₋₄) alkyl (optionally substituted by G³ and/or Z³), or the relevant pairs are linked together as hereinbefore defined;
when any pair of R^{16a} to R^{16c} and R^{17a} to R^{17f}, or R^{18a} to R^{18c} and R^{19a} to R^{19f} are linked together, they form a 5- or 6-membered ring, optionally substituted by one or more (e.g. one or two) substituents selected from G³ and/or Z³;
G³ represents halo or -A¹¹-R^{20a};
-A¹¹ represents a single bond or —O—;
Z³ represents ═O;
R^{20a}, R^{20b}, R^{20c}, R^{21a}, R^{21b}, R^{21c}, R^{21d}, R^{21e} and R^{21f} are independently selected from H, C₁₋₃ (e.g. C₁₋₂) alkyl (e.g. methyl) optionally substituted by one or more halo (e.g. fluoro) atoms, or optionally substituted aryl (e.g. phenyl), or the relevant pairs are linked together as defined herein;
when any pair of R^{20a} to R^{20c} and R^{21a} to R^{21f} are linked together, they form a 5- or 6-membered ring, optionally substituted by one or more (e.g. one or two) substituents selected from halo (e.g. fluoro) and C₁₋₂ alkyl (e.g. methyl);
R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a} and R^{25b} independently represent hydrogen or C₁₋₂ alkyl optionally substituted by ═O or, more preferably, one or more fluoro atoms;
when alkyl groups mentioned herein are substituted by halo, then that halo group is preferably fluoro.

More preferred compounds of the invention include those in which:

when ring A represents ring (I), in which there is one —N═ group present, then E^{e1}, E^{a3} or E^{a5} represents such a moiety;
when ring A represents ring (II), then W^{b} may represent —N(R^{3d})— (so forming a pyrrolyl or imidazolyl ring) or, more preferably, when Y^{b} represents —C(R^{3c})═, then W^{b} preferably represents —O— or, particularly, —S— (so forming a furanyl or, particularly, a thienyl ring) or when Y^{b} represents —N═, then W^{b} preferably represents —O— or —S— (so forming, for example, an oxazolyl or thiazolyl ring);
R^{3c} and R^{3d} independently represent H;
when ring A represents ring (III), then W^{c} preferably represents —N(R^{4d})—;
R^{4d} represents H;
R^{8c}, R^{8f} and R^{8h} independently represent H or C₁₋₃ alkyl optionally substituted by one or more fluoro atoms;
X¹, X² and X³ independently represent fluoro, chloro, —CN, methyl, ethyl, isopropyl, difluoromethyl, trifluoromethyl, methoxy, ethoxy, difluoromethoxy and/or trifluoromethoxy.

Preferred rings that ring A may represents include furyl (e.g. 2-furyl), thienyl (e.g. 2-thienyl), oxazolyl (e.g. 2-oxazolyl), thiazolyl (e.g. 2-thiazolyl), pyridyl (e.g. 2- or 4-pyridyl), pyrrolyl (e.g. 3-pyrrolyl), imidazolyl (e.g. 4-imidazolyl) or phenyl. Most preferred are phenyl and pyridyl (especially 2-pyridyl).

Preferred rings that the D₁ to D₃-containing ring may represent include 2- or 4-pyridyl (relative to the point of attachment to the —C(O)— moiety) or, preferably, phenyl.

Preferred aryl and heteroaryl groups that Y² to Y⁷ may represent include optionally substituted (i.e. by A) phenyl, naphthyl (e.g. 5,6,7,8-tetrahydronaphthyl), pyrrolyl, furyl, thienyl (e.g. 2-thienyl or 3-thienyl), imidazolyl (e.g. 2-imidazolyl or 4-imidazolyl), oxazolyl, isoxazolyl, thiazolyl, pyrazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, group. Preferred values include benzothienyl (e.g. 7-benzothienyl), 1,3-benzodioxolyl, particularly, naphthyl (e.g. 5,6,7,8-tetrahydronaphthyl or, preferably, 1-naphthyl or 2-naphthyl), more particularly, 2-benzoxazolyl, 2-benzimidazolyl, 2-benzothiazolyl, thienyl, oxazolyl, thiazolyl, pyridyl (e.g. 2- or 3-pyridyl), and, most preferably, phenyl.

Preferred substituents on Y² to Y⁷ groups include:

halo (e.g. fluoro, chloro or bromo);
cyano;
C₁₋₆ alkyl, which alkyl group may be cyclic, part-cyclic, unsaturated or, preferably, linear or branched (e.g. C₁₋₄ alkyl (such as ethyl, n-propyl, isopropyl, t-butyl or, preferably, n-butyl or methyl), all of which are optionally substituted with one or more halo (e.g. fluoro) groups (so forming, for example, fluoromethyl, difluoromethyl or, preferably, trifluoromethyl);
heterocycloalkyl, such as a 5- or 6-membered heterocycloalkyl group, preferably containing a nitrogen atom and, optionally, a further nitrogen or oxygen atom, so forming for example morpholinyl (e.g. 4-morpholinyl), piperazinyl (e.g. 4-piperazinyl) or piperidinyl (e.g. 1-piperidinyl and 4-piperidinyl) or pyrrolidinyl (e.g. 1-pyrrolidinyl), which heterocycloalkyl group is optionally substituted by one or more (e.g. one or two) substituents selected from C₁₋₃ alkyl (e.g. methyl) and ═O;

### —OR²⁶;

### —C(O)R²⁶;

### —C(O)OR²⁶;

### —N(R²⁶)R²⁷;

—S(O)ₘR²⁶ (in which m is 0 or, preferably, 1 or 2);
wherein R²⁶ and R²⁷ independently represent, on each occasion when used herein, H, C₁₋₆ alkyl, such as C₁₋₄ alkyl (e.g. ethyl, n-propyl, t-butyl or, preferably, n-butyl, methyl or isopropyl) optionally substituted by one or more halo (e.g. fluoro) groups (so forming e.g. a perfluoroethyl or, preferably, a trifluoromethyl group) or aryl (e.g. phenyl) optionally substituted by one or more halo or C₁₋₃ (e.g. C₁₋₂) alkyl groups (which alkyl group is optionally substituted by one or more halo (e.g. fluoro) atoms). Preferably, when the substituent is —S(O)R²⁶ or —S(O)₂R²⁶, then R²⁶ does not represent hydrogen.

Preferred compounds of the invention include those in which:

D₁, D₂ and D₃ respectively represent —C(R^{1a})═, —C(R^{1b})═ and —C(R^{1c})═;
R^{1a}, R^{1b} and R^{1c} independently represent hydrogen;
ring A represents ring (I);
E^{a1} and E^{a5} independently represent —C(H)═ or either one of these represents —N═;
E^{a1} and E^{a5} independently represent —C(H)═;
E^{2a}, E^{a3} and E^{a4} respectively represent —C(R^{2b})═, —C(R^{2c})═ and —C(R^{2d})═;
R^{2b} represents -L^{1a}-Y^{1a} or, preferably, H;
R^{2c} represents the requisite -L²-Y² group;
R^{2d} represents H;
T represents T², preferably, T⁴ and, especially, T¹ or T⁵;
W¹ represents —N—;
E^{d1} represents —C(R¹)═ or, preferably, —N═;
E^{d1} represents —N═;
E^{d3} represents —C(R¹)═ or —C(Y³)═;
W² represents —C(R¹)═ or —C(Y³)═;
one of E^{d3} and W² (preferably E^{d3}) represents —C(R¹)═ and the other represents —C(Y³)═;
when T represents T¹, then the requisite Y³ or Y⁴ group is preferably in the ortho position, i.e. W² preferably represents —C(Y³)═;
Y² and Y³, Y⁴, Y⁵, Y⁶ or Y⁷ (preferably Y² and Y³) both represent aryl or heteroaryl optionally substituted as defined herein;
preferred T¹ groups include 1-triazolyl groups, substituted with a Y³ or Y⁴ (as appropriate) group at the 4- or, preferably, 5-position;
W³ represents —N—;
E^{h2} represents —N═;
E^{h1}, E^{h3}, E^{h4}, E^{h5}, E^{h6}, E^{h7} and E^{h8} independently represent —C(R¹)═;
preferred T⁵ groups include 1-benzimidazolyl groups;
R¹ represents hydrogen or R^{25a};
R^{25a} represents C₁₋₃ (e.g. C₁₋₂) alkyl (e.g. methyl);
Y represents —C(O)—;
there are no -L^{1a}-Y^{1a} groups present;
L¹ and L^{1a} independently represent a single bond;
Y¹ represents —C(O)OR^{6a};
R^{6a} represents hydrogen;
L² represents —OA¹⁷- or, preferably, —N(R^{17a})-A¹⁶-;
A¹⁶ represents a direct bond, —C(O)— or —S(O)₂—;
when L² represents —N(R^{17a})-A¹⁶-, then A¹⁶ preferably represents a direct bond;
A¹⁷ represents a direct bond;
R^{17a} represents hydrogen or C₁₋₆ alkyl optionally substituted by one or more (e.g. one) substituent(s) selected from —OCH₃ and —CN (preferably R^{17a} is methyl);
when R^{17a} represents optionally substituted C₁₋₆ alkyl, then that group may represent: a linear unsaturated C₁₋₆ (e.g. C₁₋₄, such as C₁₋₃) alkyl group (e.g. methyl, ethyl or propyl) optionally substituted by —OCH₃ and/or —CN, so forming for example a methoxyethyl (i.e. —(CH₂)₂—OCH₃), ethoxyethyl or cyanopropyl (i.e. —(CH₂)₃—CN); a part cyclic C₁₋₆ alkyl group (for example C₁₋₂ alkyl (e.g. methyl) substituted by C₃₋₅ cycloalkyl), such as cyclopropylmethyl (i.e. —CH₂-cyclopropyl), cyclobutylmethyl or cyclopentylmethyl; a linear saturated C₁₋₆ (e.g. C₁₋₄, such as C₁₋₃) alkyl group (in which the unsaturation is preferably one double or one triple bond), such as allyl (i.e. —CH₂—CH═CH) or propynyl (i.e. —CH₂—CH≡CH);
A represents G¹ or C₁₋₆ (e.g. C₁₋₄) alkyl (e.g. butyl (such as n-butyl) or methyl) optionally substituted by one or more substituents selected from G¹;
G¹ represents halo (e.g. chloro or fluoro; for example, when attached to an aromatic ring, the halo group may be chloro or fluoro, and when attached to a non-aromatic group, such as alkyl, then the halo group is preferably fluoro) or -A¹-R^{16a};
A¹ represents a single bond or, preferably, —OA⁵-;
A⁵ represents a single bond;
R^{16a} represents hydrogen or C₁₋₆ (e.g. C₁₋₄) alkyl optionally substituted by one or more substituents selected from G³ (e.g. R^{16a} may represent ethyl or, preferably, butyl (such as tert-butyl or, preferably n-butyl), propyl (such as isopropyl) or methyl);
G³ represents halo (e.g. fluoro; and hence e.g. R^{16a} may represent trifluoromethyl or perfluoroethyl);
when Y² represents an optionally substituted phenyl group, then that phenyl group may be substituted with two substituents (e.g. with one at the para-position and the other at the meta- or ortho- (3- or 2-) position, so forming for example a 3,4-substituted, 2,4-substituted or 2,5-substituted phenyl group) or, preferably, with a single substituent (e.g. at the para- (or 4-) position);
R²⁸ represents hydrogen or unsubstituted C₁₋₃ (e.g. C₁₋₂) alkyl (e.g. methyl).

Preferred substituents on Y² to Y⁷ groups (for instance, when they represent heteroaryl groups or, preferably, aryl group, such as phenyl) include halo (e.g. chloro) atoms.

Particularly preferred compounds of the invention include those of the following formula:

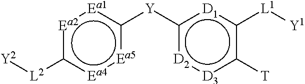

wherein
Y represents —C(O)— or —C(═N—OR²⁸)—;
R²⁸ represents hydrogen or C₁₋₄ (e.g. C₁₋₂) alkyl;
D₁, D₂ and D₃ respectively represent —C(R^{1a})═, —C(R^{1b})═ and —C(R^{1c})═;
R^{1b} and R^{1c} independently represent R^{5a}, halo, —CN or, preferably, hydrogen;
at least one (e.g. at least two, and preferably all) of R^{1a}, R^{1b} and R^{1c} represent hydrogen;
each of E^{a1}, E^{a2}, E^{a4} and E^{a5} respectively represent —C(H)═, —C(R^{2b})═, —C(R^{2d})═ and —C(H)═, or, one or two (e.g. one) of E^{a1}, E^{a2}, E^{a4} and E^{a5} (e.g. E^{a1} and/or E^{a5}; preferably either E^{a1} or E^{a5}) may alternatively and independently represent —N═ (hence, this ring is preferably phenyl, pyridyl, such as 2-pyridyl, or pyrimidinyl, such as 2-pyrimidinyl; most preferably the E^{a1} to E^{a5}-containing ring represents phenyl or 2-pyridyl);
R^{2b} and R^{2d} independently represent a substituent selected from X¹ or, more preferably, hydrogen;
preferably, R^{2b}, R^{2c} and R^{2d} do not represent a substituent -L^{1a}-Y^{1a} (e.g. a carboxylic acid or ester thereof);
X¹, X² and X³ independently represent a group selected from R^{5a}, halo or —CN;
R^{5a} represents, on each occasion when used herein, C₁₋₆ (e.g. C₁₋₄) alkyl optionally substituted by one or more substituents selected from ═O and, preferably, halo, —CN and —N₃ (e.g. halo and —CN);
Y¹ and Y^{1a} independently represent, on each occasion when used herein, —C(O)OR^{9a};
R^{9a} represents hydrogen or C₁₋₆ (e.g. C₁₋₄) alkyl;
T represents one of the following structures (i.e. T may represent T⁷ or, preferably, T¹, T² or T⁵):

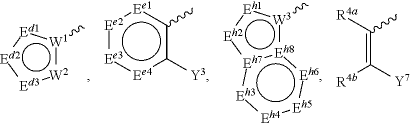

W¹ represents —N— or —C═;
E^{d1} represents —N═ or —C(R¹)═;
E^{d2} represents —N═, —C(R¹)═ or —C(Y³)═;
E^{d3} represents —N═, —C(Y³)═ (e.g. —C(phenyl)═ or —C(pyridyl)═) or —C(R¹)═(e.g. —C(H)═);
W² represents —C(Y³)═ (e.g. —C(phenyl)═ or —C(pyridyl)═) or —C(R¹)═(e.g. —C(H)═) or, when W¹ represents —C—, then W² may represent —N(Y⁴)═;
when E^{d2} represents —C(Y³)=(and e.g. W¹ and E^{d1} represent —N— and —N═, respectively), then preferably E^{d3} and W² independently represent —C(R¹)═;
when W¹ represents —N— and E^{d3} represents —N═, then W² preferably represents —C(Y³)=(and preferably, E^{d1} and E^{d2} independently represent —C(R¹)═);
when W¹ represents —N—, then preferably:
(i) E^{d1} represents —N═;
E^{d2} represents —N═ or —C(R¹)═; and
one of E^{d3} and W² represents —C(Y³)═ (e.g. —C(phenyl)═ or —C(pyridyl)═) and the other represents —C(R¹)═(e.g. —C(H)═); or
(ii) E^{d1} represents —N═;
E^{d2} represents —C(Y³)═; and
E^{d3} and W² independently represent —C(R¹)═; or
(iii) E^{d1} represents —C(R¹)═;
E^{d2} represents —C(R¹)═;
E^{d3} represents —N═ or —C(R¹)═;
W² represents —C(Y³)═;
when W¹ represents —C═, then preferably:
E^{d1} represents —N═;
E^{d2} and E^{d3} independently represent —C(R¹)═;
W² represents —C(Y⁴)═;
E^{e1}, E^{e2}, E^{e3} and E^{e4} independently represent —C(R¹)═(in which each R¹ is preferably hydrogen);
W³ represents —N—;
E^{h2} represents —N—;
E^{h1} represents —C(R¹)═;
E^{h3}, E^{h4}, E^{h5} and E^{h6} independently represent —C(R¹)═;
R^{4a} and R^{4b} independently represent hydrogen;
Y², Y³, Y⁴ and Y⁷ independently represent aryl (e.g. phenyl) or pyridyl (e.g. 2-pyridyl), both of which are optionally substituted by one or more substituents selected from A (e.g. G¹ or C₁₋₆ alkyl optionally substituted by one or more substituents selected from G¹);
Y⁴ most preferably represents aryl (e.g. phenyl);
Y⁷ represents aryl (e.g. phenyl);
for instance when W¹ represents —N—, T¹ preferably represent:

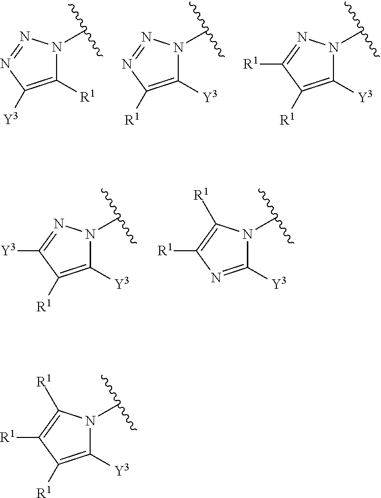

for instance when W¹ represents —C═, T¹ preferably represents:

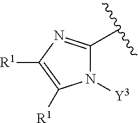

for instance, T² preferably represents:

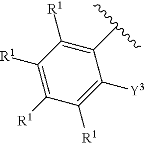

for instance, T⁵ preferably represents:

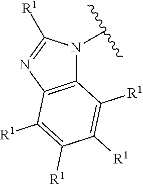

each R¹ independently represents halo, —CN or, preferably hydrogen or R^{25a};
R^{25a} represents C₁₋₆ (e.g. C₁₋₄) alkyl (e.g. methyl) optionally substituted by one or more fluoro substituents (so forming e.g. a trifluoromethyl group);
Y² represents aryl (e.g. phenyl) optionally substituted by one or more substituents selected from A;
A represents G¹ or C₁₋₆ alkyl optionally substituted by one or more substituents selected from G¹ (A more preferably represents G¹);
G¹ represents halo (e.g. chloro or fluoro);
Y¹ and Y^{1a} independently represent —C(O)OR^{9a};
R^{9a} represents hydrogen or C₁₋₆ (e.g. C₁₋₄) alkyl;
L¹ and L^{1a} independently represent a single bond;
L² represents —N(R^{17a})-A¹⁶-;
A¹⁶ represents a direct bond;
R^{17a} represents hydrogen or C₁₋₆ (e.g. C₁₋₄, such as C₁₋₂) alkyl (e.g. methyl or cyclopropylmethyl).

For the avoidance of doubt, all individual features (e.g. preferred features) mentioned herein may be taken in isolation or in combination with any other feature (including preferred feature) mentioned herein (hence, preferred features may be taken in conjunction with other preferred features, or independently of them).

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) for compounds of formula I in which Y represents —C(O)—, oxidation of a compound of formula II,

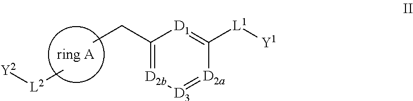

wherein ring A, D₁, D₂ₐ, D_{2b}, D₃, L¹, Y¹, L² and Y² are as hereinbefore defined, in the presence of a suitable oxidising agent, for example, KMnO₄, optionally in the presence of a suitable solvent, such as acetone, and an additive such as magnesium sulfate;
(ia) for compounds of formula I in which Y represents —C(O)—, oxidation of a compound of formula IIA,

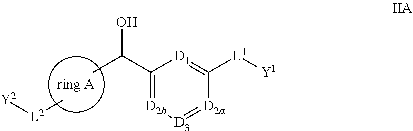

wherein ring A, D₁, D₂ₐ, D_{2b}, D₃, L¹, Y¹, L² and Y² are as hereinbefore defined, in the presence of a suitable oxidising agent, for example, pyridinium chlorochromate (PCC) or the like (e.g. pyridinium dichromate; PDC);
(ii) for compounds of formula I in which L² represents —N(R^{17a})A¹⁶- in which R^{17a} represents H (and, preferably, Y is —C(O)— or R²⁸ is C₁₋₆ alkyl optionally substituted by one or more halo atoms), reaction of a compound of formula III,

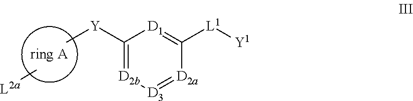

or a protected derivative thereof (e.g. an amino-protected derivative or a keto-protecting group, such as a ketal or thioketal) wherein L^{2a} represents —NH₂, and Y, ring A, D₁, D₂ₐ, D_{2b}, D₃, L¹ and Y¹ are as hereinbefore defined, with:
(A) when A¹⁶ represents —C(O)N(R^{17b})—, in which R^{17b} represents H:
(a) a compound of formula IV,

Y^{a}—N═C═O IV;
or
(b) with CO (or a reagent that is a suitable source of CO (e.g. Mo(CO)₆ or CO₂(CO)₈)) or a reagent such as phosgene or triphosgene in the presence of a compound of formula V,

Y²—NH₂ V

wherein, Y² is as hereinbefore defined. For example, in the case of (a) above, in the presence of a suitable solvent (e.g. THF, dioxane or diethyl ether) under reaction conditions known to those skilled in the art (e.g. at room temperature). In the case of (b), suitable conditions will be known to the skilled person, for example the reactions may be carried out in the presence of an appropriate catalyst system (e.g. a palladium catalyst), preferably under pressure and/or under microwave irradiation conditions. The skilled person will appreciate that the compound so formed may be isolated by precipitation or crystallisation (from e.g. n-hexane) and purified by recrystallisation techniques (e.g. from a suitable solvent such as THF, hexane (e.g. n-hexane), methanol, dioxane, water, or mixtures thereof). Protection (at e.g. an amino group) followed by deprotection may be necessary, or the reaction may be performed with less than 2 equivalents of the compound of formula IV or V (as appropriate);
(B) when A¹⁶ represents a single bond, with a compound of formula VI,

Y²-L^{a} VI

wherein L^{a} represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂ (or a protected derivative thereof, e.g. an alkyl protected derivative, so forming, for example a 4,4,5,5-tetramethyl-1,3,2-dioxaborolan-2-yl group) and Y² is as hereinbefore defined, for example optionally in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent (e.g. when Y² represents phenyl and L^{a} represents bromo, i.e. bromobenzene). This reaction may be carried out at room temperature or above (e.g. at a high temperature, such as the reflux temperature of the solvent system that is employed) or using microwave irradiation;
(C) when A¹⁶ represents —S(O)₂—, —C(O)— or —C(O)—C(R^{y6})(R^{y7})—, with a compound of formula VII,

Y²-A^{16a}-L^{a} VII

wherein A^{16a} represents —S(O)₂—, —C(O)— or —C(O)—C(R^{y6})(R^{y7})—, and Y² and L^{a} are as hereinbefore defined, and L^{a} is preferably, bromo or chloro, under reaction conditions known to those skilled in the art, the reaction may be performed at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile, dimethylformamide, trifluoromethylbenzene, dioxane or triethylamine);
(iii) for compounds of formula I in which one of L² represents —N(R^{17a})C(O)N(R^{17b})—, in which R^{17a} and R^{17b} both represent H, and, preferably, Y is —C(O)— or R²⁸ is C₁₋₆ alkyl optionally substituted by one or more halo atoms, reaction of a compound of formula VIII,

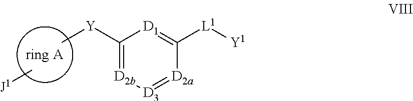

wherein J¹ represents —N═C═O, and Y, ring A, D₁, D₂ₐ, D_{2b}, D₃, L¹ and Y¹ are as hereinbefore defined, with a compound of formula V as hereinbefore defined, under reaction conditions known to those skilled in the art, such as those described hereinbefore in respect of process step (ii)(A)(b) above;
(iv) for compounds of formula I in which, preferably, Y is —C(O)— or R²⁸ is C₁₋₆ alkyl optionally substituted by one or more halo atoms, reaction of a compound of formula IX,

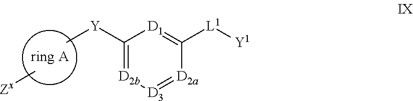

wherein Z^{x} represents a suitable leaving group, in which the suitable leaving group may be fluoro or, preferably, chloro, bromo, iodo, a sulfonate group (e.g. —OS(O)₂CF₃, —OS(O)₂CH₃, —OS(O)₂PhMe or a nonaflate), —B(OH)₂, —B(OR^{wx})₂, —Sn(R^{wx})₃ or diazonium salts, in which each R^{wx} independently represents a C₁₋₆ alkyl group, or, in the case of —B(OR^{wx})₂, the respective R^{wx} groups may be linked together to form a 4- to 6-membered cyclic group (such as a 4,4,5,5-tetramethyl-1,3,2-dioxaborolan-2-yl group), or, alternatively, Z^{x} may represent —N₃, and Y, ring A, D₁, D₂ₐ, D_{2b}, D₃, L¹ and Y¹ are as hereinbefore defined, with a (or two separate) compound(s) (as appropriate/required) of formula X,

Y²-L²-H X

wherein L² and Y² is as hereinbefore defined, under suitable reaction conditions known to those skilled in the art, for example such as those hereinbefore described in respect of process (ii) above (e.g. (II)(B)), for example optionally in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof). Alternatively, for example, when L² represents —O— or —S— (and hence the compound of formula X is an alcohol, e.g. a phenol or a thiol, e.g. thiophenol), or, L² represent a single bond, and Y² is to be attached to the requisite biaryl moiety (of the compounds of the invention, which may alternatively be termed the diaryl; for the purposes herein both terms may be interchangeably employed) via a heteroatom, e.g. nitrogen), the reaction may be performed in the presence of a mixture of KF/Al₂O₃ (e.g. in the presence of a suitable solvent such as acetonitrile, at elevated temperature, e.g. at about 100° C.; in this instance the leaving group that Z^{x} may represent in the compound of formula IX is preferably fluoro). Alternatively, when Z^{x} represents —N₃, and the compound to be formed is one in which L² is a single bond and Y² is a triazolyl group, then the reaction is performed in the presence of an appropriate alkynyl compound (optionally in the presence of Cp*RuClCOD; depending on the regioselectivity of the compound to be prepared), for example, when a 4-phenyl- or 5-phenyl-1-triazolyl group is required, then the appropriate alkynyl compound is 1-ethynylbenzene (which may result in the formation of the 4-phenyl-1-triazolyl in the absence of Cp*RuClCOD, but in the presence thereof may result in the formation of the 5-phenyl-1-triazolyl);
(iva) for compounds of formula I in which, preferably, Y is —C(O)— or R²⁸ is C₁₋₆ alkyl optionally substituted by one or more halo atoms, reaction of a compound of formula IXA,

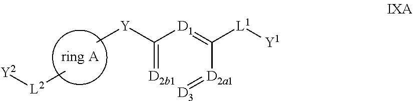

wherein one of D^{2b1} and D^{2a1} represents —C(—Z^{y})═, and the other represents D₂, Z^{y} represents (independently) a group defined in respect of Z^{x} or, alternatively, Z^{y} may represent —N₃, and Y, ring A, D₁, D₂ₐ, D_{2b}, D₃, L¹, Y¹, L² and Y² are as hereinbefore defined, with a compound of formula XA,

T-H XA

wherein T is as hereinbefore defined, under suitable reaction conditions known to those skilled in the art, for example such as those hereinbefore described in respect of process (iv) above. Preferably, the T group to be attached to the D₁ to D₃-containing ring is to be attached via a heteroatom, e.g. nitrogen), in which case the reaction is preferably performed in the presence of a mixture of KF/Al₂O₃ (e.g. in the presence of a suitable solvent such as acetonitrile, at elevated temperature, e.g. at about 100° C.; in this instance the leaving group that Z^{y} may represent in the compound of formula IXA is preferably fluoro). Alternatively, when Z^{y} represents —N₃, and the compound to be formed is one in which T is a triazolyl group, then the reaction is performed in the presence of an appropriate alkynyl compound (optionally in the presence of Cp*RuClCOD; depending on the regioselectivity of the compound to be prepared), for example, when a 4-phenyl- or 5-phenyl-1-triazolyl group is required, then the appropriate alkynyl compound is 1-ethynylbenzene (which may result in the formation of the 4-phenyl-1-triazolyl in the absence of Cp*RuClCOD, but in the presence thereof may result in the formation of the 5-phenyl-1-triazolyl);
(v) compounds of formula I in which there is a R^{17a} or R^{17b} group present that does not represent hydrogen (or if there is R⁵, R⁶, R⁷, R⁸, R⁹, R¹¹, R¹², R¹³, R¹⁴, R¹⁶, R¹⁷ or R¹⁸ group present, which is attached to a heteroatom such as nitrogen or oxygen, and which does/do not represent hydrogen), may be prepared by reaction of a corresponding compound of formula I in which such a group is present that does represent hydrogen with a compound of formula XI,

R^{wy}-L^{b} XI

wherein R^{wy} represents either R^{17a} or R^{17b} (as appropriate) as hereinbefore defined provided that it does not represent hydrogen (or R^{wy} represents a R⁵ to R¹⁸ group in which those groups do not represent hydrogen), and L^{b} represents a suitable leaving group such as one hereinbefore defined in respect of L^{a} or —Sn(alkyl)₃ (e.g. —SnMe₃ or —SnBu₃), or a similar group known to the skilled person, under reaction conditions known to those skilled in the art, for example such as those described in respect of process step (ii)(C) above. The skilled person will appreciate that various groups (e.g. primary amino groups) may need to be mono-protected and then subsequently deprotected following reaction with the compound of formula XI;
(vi) for compounds of formula I that contain only saturated alkyl groups, reduction of a corresponding compound of formula I that contains an unsaturation, such as a double or triple bond, in the presence of suitable reducing conditions, for example by catalytic (e.g. employing Pd) hydrogenation;
(vii) for compounds of formula I in which Y¹ and/or, if present, Y^{1a} represents —C(O)OR^{9a}, in which R^{9a} represent hydrogen (or other carboxylic acid or ester protected derivatives (e.g. amide derivatives)), hydrolysis of a corresponding compound of formula I in which R^{9a} does not represent H, under standard conditions, for example in the presence of an aqueous solution of base (e.g. aqueous 2M NaOH) optionally in the presence of an (additional) organic solvent (such as dioxane or diethyl ether), which reaction mixture may be stirred at room or, preferably, elevated temperature (e.g. about 120° C.) for a period of time until hydrolysis is complete (e.g. 5 hours). Alternatively, non-hydrolytic means may be employed to convert esters to acids e.g. by hydrogentation or oxidation (e.g. for certain benzylic groups) known to those skilled in the art;
(viii) for compounds of formula I in which Y¹ and/or, if present, Y^{1a} represents —C(O)OR^{9a} and R^{9a} does not represent H:
(A) esterification (or the like) of a corresponding compound of formula I in which R^{9a} represents H; or
(B) trans-esterification (or the like) of a corresponding compound of formula I in which R^{9a} does not represent H (and does not represent the same value of the corresponding R^{9a} group in the compound of formula I to be prepared),
   under standard conditions in the presence of the appropriate alcohol of formula XII,

R^{9za}OH XII

in which R^{9za} represents R^{a} provided that it does not represent H, for example further in the presence of acid (e.g. concentrated H₂SO₄) at elevated temperature, such as at the reflux temperature of the alcohol of formula XII;
(ix) for compounds of formula I in which Y¹ and/or, if present, Y^{1a} represents —C(O)OR^{9a}, in which R^{9a} is other than H, and L¹ is as hereinbefore defined, provided that it does not represent —(CH₂)ₚ-Q-(CH₂)_{q}— in which p represents 0 and Q represents —O—, and, preferably, Y is —C(O)— or R²⁸ is C₁₋₆ alkyl optionally substituted by one or more halo atoms, reaction of a compound of formula XIII,

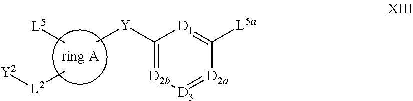

wherein at least one of L⁵ and L^{5a} represents an appropriate alkali metal group (e.g. sodium, potassium or, especially, lithium), a —Mg-halide, a zinc-based group or a suitable leaving group such as halo or —B(OH)₂, or a protected derivative thereof (e.g. an alkyl protected derivative, so forming for example a 4,4,5,5-tetramethyl-1,3,2-dioxaborolan-2-yl group), and the other may represent -L¹-Y¹ or -L^{1a}-Y^{1a} (as appropriate), and Y, ring A, D₁, D₂ₐ, D_{2b}, D₃, L² and Y² are as hereinbefore defined (the skilled person will appreciate that the compound of formula XIII in which L⁵ and/or L^{5a} represents an alkali metal (e.g. lithium), a Mg-halide or a zinc-based group may be prepared from a corresponding compound of formula XIII in which L⁵ and/or L^{5a} represents halo, for example under conditions such as Grignard reaction conditions, halogen-lithium exchange reaction conditions, which latter two may be followed by transmetallation, all of which reaction conditions are known to those skilled in the art), with a compound of formula XIV,

L⁶-L^{xy}-Y^{b} XIV

wherein L^{xy} represents L¹ or L^{1a} as hereinbefore defined (provided that it does not represent —(CH₂)ₚ-Q-(CH₂)_{q}— in which p represents 0 and Q represents —O—) and Y^{b} represents —C(O)OR^{9a}, in which R^{9a} is other than H, and L⁶ represents a suitable leaving group known to those skilled in the art, such as C₁₋₃ alkoxy and, preferably, halo (especially chloro or bromo). For example, the compound of formula XIV may be Cl—C(O)OR^{9a}. The reaction may be performed under standard reaction conditions, for example in the presence of a polar aprotic solvent (e.g. THF or diethyl ether);
(x) compounds of formula I in which L¹ preferably represents a single bond, and Y¹ represents 5-tetrazolyl (and, preferably, Y is —C(O)— or R²⁸ is C₁₋₆ alkyl optionally substituted by one or more halo atoms), or, L^{1a} and Y^{1a}, if present, represent those groups defined by L¹ and Y¹, may be prepared in accordance with the procedures described in international patent application WO 2006/077366;
(xi) for compounds of formula I in which L¹ and/or, if present, L^{1a} represent a single bond, and Y¹ and/or, if present, Y^{1a} represent —C(O)OR^{9a} in which R^{9a} is H, (and, preferably, Y is —C(O)— or R²⁸ is C₁₋₆ alkyl optionally substituted by one or more halo atoms), reaction of a compound of formula XIII as hereinbefore defined but in which L^{6a} represents either:
(I) an alkali metal (for example, such as one defined in respect of process step (ix) above); or
(II) —Mg-halide,
   with carbon dioxide, followed by acidification under standard conditions known to those skilled in the art, for example, in the presence of aqueous hydrochloric acid;
   (xii) for compounds of formula I in which L¹ and/or, if present, L^{1a} represent a single bond, and Y¹ and/or, if present, Y^{1a} represent —C(O)OR^{9a} (and, preferably, Y is —C(O)— or R²⁸ is C₁₋₆ alkyl optionally substituted by one or more halo atoms), reaction of a corresponding compound of formula XIII as hereinbefore defined but in which L^{5a} is a suitable leaving group known to those skilled in the art (such as a sulfonate group (e.g. a triflate) or, preferably, a halo (e.g. bromo or iodo) group) with CO (or a reagent that is a suitable source of CO (e.g. Mo(CO)₆ or CO₂(CO)₈)), in the presence of a compound of formula XV,

R^{9a}OH XV

wherein R^{9a} is as hereinbefore defined, and an appropriate catalyst system (e.g. a palladium catalyst, such as PdCl₂, Pd(OAc)₂, Pd(Ph₃P)₂Cl₂, Pd(Ph₃P)₄, Pd₂(dba)₃ or the like) under conditions known to those skilled in the art;
(xiii) for compounds of formula I in which Y represents —C(O)—, reaction of either a compound of formula XVI or XVII,

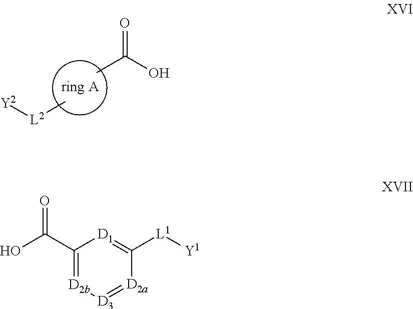

respectively with a compound of formula XVIII or XIX,

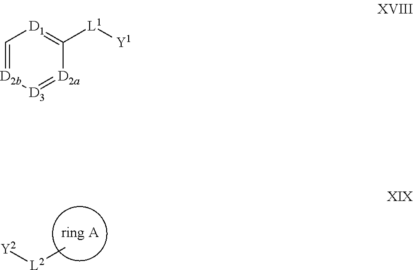

wherein (in all cases) ring A, D₁, D₂ₐ, D_{2b}, D₃, L¹, Y¹, L² and Y² are as hereinbefore defined, in the presence of a suitable reagent that converts the carboxylic acid group of the compound of formula XVI or XVII to a more reactive derivative (e.g. an acid chloride or acid anhydride, or the like; which reactive derivative may itself be separately prepared and/or isolated, or where such a reactive derivative may be prepared in situ) such as POCl₃, in the presence of ZnCl₂, for example as described in Organic and Biomolecular Chemistry (2007), 5(3), 494-500 or, more preferably, PCl₅, PCl₅, SOCl₂ or (COCl)₂. Alternatively, such a reaction may be performed in the presence of a suitable catalyst (for example a Lewis acid catalyst such as SnCl₄), for example as described in Journal of Molecular Catalysis A: Chemical (2006), 256(1-2), 242-246 or under alternative Friedel-crafts acylation reaction conditions (or variations thereupon) such as those described in Tetrahedron Letters (2006), 47(34), 6063-6066; Synthesis (2006), (21), 3547-3574; Tetrahedron Letters (2006), 62(50), 11675-11678; Synthesis (2006), (15), 2618-2623; Pharmazie (2006), 61(6), 505-510; and Synthetic Communications (2006), 36(10), 1405-1411. Alternatively, such a reaction between the two relevant compounds may be performed under coupling reaction conditions (e.g. Stille coupling conditions), for example as described in Bioorganic and Medicinal Chemistry Letters (2004), 14(4), 1023-1026;
(xiv) for compounds of formula I in which Y represents —C(O)—, reaction of either a compound of formula XX or XXI,

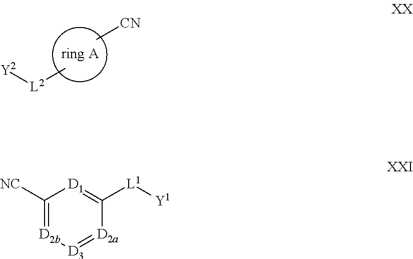

with a compound of formula XXII or XXIII,

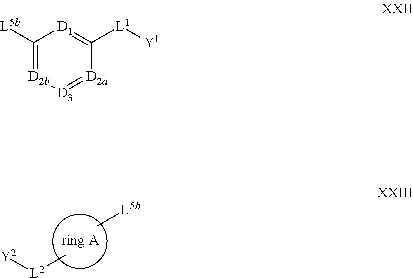

respectively, wherein L^{5b} represents L⁵ as hereinbefore defined provided that it does not represent -L¹-Y¹, and which L^{5b} group may therefore represents —B(OH)₂ (or a protected derivative thereof), an alkali metal (such as lithium) or a —Mg-halide (such as —MgI or, preferably, —MgBr), and (in all cases) ring A, D₁, D₂ₐ, D_{2b}, D₃, L¹, Y¹, C and Y² are as hereinbefore defined, and (in the case of compounds of formulae XXII and XXIII), for example in the presence of a suitable solvent, optionally in the presence of a catalyst, for example, as described in Organic Letters (2006), 8(26), 5987-5990. Compounds of formula I may also be obtained by performing variations of such a reaction, for example by performing a reaction of a compound of formula XX or XXI respectively with a compound of formula XVIII or XIX as hereinbefore defined, for example under conditions described in Journal of Organic Chemistry (2006), 71(9), 3551-3558 or US patent application US 2005/256102;
(xv) for compounds of formula I in which Y represents —C(O)—, reaction of an activated derivative of a compound of formula XVI or XVII as hereinbefore defined (for example an acid chloride; the preparation of which is hereinbefore described in process step (xiii) above), with a compound of formula XXII or XXIII (as hereinbefore defined), respectively, for example under reaction conditions such as those hereinbefore described in respect of process step (xiv) above;
(xvi) for compounds of formula I in which Y represents —C(═N—OR²⁸)—, reaction of a corresponding compound of formula I, with a compound of formula XXIIIA,

H₂N—O—R²⁸ XXIIIA

wherein R²⁸ is represents hydrogen or C₁₋₆ alkyl optionally substituted by one or more halo atoms, under standard condensation reaction conditions, for example in the presence of an anhydrous solvent (e.g. dry pyridine, ethanol and/or another suitable solvent);
(xvii) for compounds of formula I in which Y represents —C(═N—OR²⁸)— and R²⁸ represents C₁₋₆ alkyl optionally substituted by one or more halo atoms, reaction of a corresponding compound of formula I, in which R²⁸ represents hydrogen, with a compound of formula XXIIIB,

R^{28a}-L⁷ XXIIIB

wherein R^{28a} represents R²⁸, provided that it does not represent hydrogen and L⁷ represents a suitable leaving group, such as one hereinbefore defined in respect of L^{a} (e.g. bromo or iodo), under standard alkylation reaction conditions, such as those hereinbefore described in respect of process step (ii).

Compounds of formula II may be prepared by reaction of a compound of formula XVIII with a compound of formula XIX, both as hereinbefore defined, with formaldehyde (e.g. in the form of paraformaldehyde or an aqueous solution of formaldehyde such as a 3% aqueous solution), for example under acidic conditions (e.g. in the presence of aqueous HCl) at or above room temperature (e.g. at between 50° C. and 70° C.). Preferably, the formaldehyde is added (e.g. slowly) to an acidic solution of the compound of formula XVIII at about 50° C., with the reaction temperature rising to about 70° C. after addition is complete. When acidic conditions are employed, precipitation of the compound of formula II may be effected by the neutralisation (for example by the addition of a base such as ammonia). Compounds of formula I may also be prepared in accordance with such a procedure, for example under similar reaction conditions, employing similar reagents and reactants.

Compounds of formula IIA may be prepared by reaction of a compound of formula XXIIIC or XXIIID,

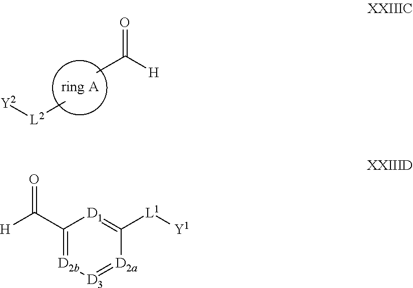

wherein ring A, D₁, D₂ₐ, D_{2b}, D₃, L¹, L², Y¹ and Y² are as hereinbefore defined, with a compound of formula XXII or XXIII, respectively, for example under reaction conditions such as those hereinbefore described in respect of preparation of compounds of formula I (process step (xiii)).

Compounds of formulae III, VIII, IX and XIII in which Y represents —C(O)—, may be prepared by oxidation of a compound of formulae XXIV, XXV, XXVI and XXVII, respectively,

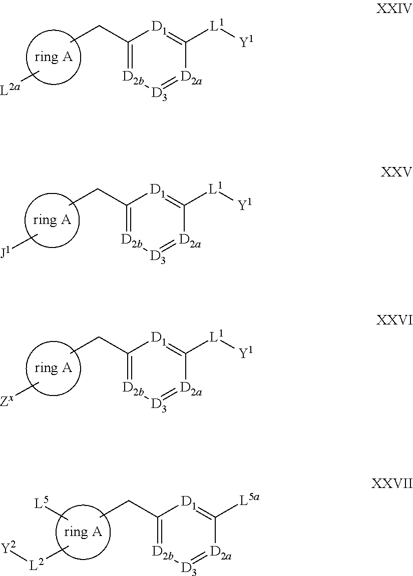

wherein ring A, D₁, D₂ₐ, D_{2b}, D₃, L¹, Y¹, Z^{x}, L², Y², J¹, L⁵ and L^{5a} are as hereinbefore defined, under standard oxidation conditions known to those skilled in the art, for example such as those hereinbefore described in respect of preparation of compounds of formula I (process step (i) above). The skilled person will appreciate that, similarly, compounds of formulae XXIV, XXV, XXVI and XXVII may be prepared by reduction of corresponding compounds of formulae III, VIII, IX and XIII, under standard reaction conditions, such as those described herein.

Compounds of formula III in which Y represents —C(O)—, or, preferably, XXIV (or protected, e.g. mono-protected derivatives thereof) may be prepared by reduction of a compound of formula XXVIII,

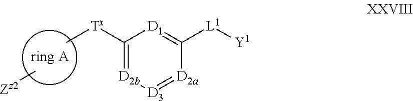

wherein T^{x} represents —C(O)— (in the case where compounds of formula III are to be prepared) or, preferably, —CH₂— (in the case where compounds of formula XXIV are to be prepared), in which Z^{z2} represents —N₃ or —NO₂, under standard reaction conditions known to those skilled in the art, in the presence of a suitable reducing agent, for example reduction by catalytic hydrogenation (e.g. in the presence of a palladium catalyst in a source of hydrogen) or employing an appropriate reducing agent (such as trialkylsilane, e.g. triethylsilane). The skilled person will appreciate that where the reduction is performed in the presence of a —C(O)— group (e.g. when T^{x} represents —C(O)—), a chemoselective reducing agent may need to be employed.

Compounds of formula III in which L^{2a} represent —NH₂ (or protected derivatives thereof) may also be prepared by reaction of a compound of formula IX as defined above, with ammonia, or preferably with a protected derivative thereof (e.g. benzylamine or Ph₂C═NH), under conditions such as those described hereinbefore in respect of preparation of compounds of formula I (process step (iv) above).

Compounds of formulae III, IX, XXIV or XXVI in which L¹ represents a single bond, and Y¹ represents —C(O)OR^{9a}, may be prepared by:

(I) reaction of a compound of formula XXIX,

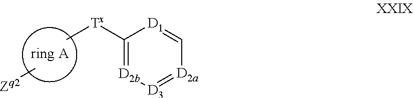

wherein Z^{q2} represent Z^{x} (in the case of preparation of compounds of formulae IX or XXVI) or L^{2a} (in the case of preparation of compounds of formulae III or XXIV), and ring A, D₁, D₂ₐ, D_{2b}, D₃, Z^{x}, L^{2a} and T^{x} are as hereinbefore defined, with a suitable reagent such as phosgene or triphosgene in the presence of a Lewis acid, followed by reaction in the presence of a compound of formula XV as hereinbefore defined, hence undergoing a hydrolysis or alcoholysis reaction step;
(II) for such compounds in which R^{9a} represents hydrogen, formylation of a compound of formula XXIX as hereinbefore defined, for example in the presence of suitable reagents such as P(O)Cl₃ and DMF, followed by oxidation under standard conditions;
(III) reaction of a compound of formula XXX,

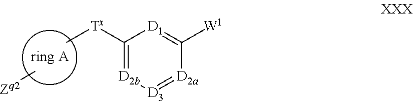

wherein W¹ represents a suitable leaving group such as one defined by Z^{x} above, and ring A, D₁, D₂ₐ, D_{2b}, D₃, Z^{q2} and T^{x} are as hereinbefore defined, are as hereinbefore defined, with CO (or a reagent that is a suitable source of CO (e.g. Mo(CO)₆ or CO₂(CO)₈) followed by reaction in the presence of a compound of formula XV as hereinbefore defined, under reaction conditions known to those skilled in the art, for example such as those hereinbefore described in respect of preparation of compounds of formula I (process step (ii), e.g. (ii)(A)(b), above), e.g. the carbonylation step being performed in the presence of an appropriate precious metal (e.g. palladium) catalyst;
(IV) reaction of a compound of formula XXXI,

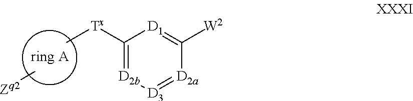

wherein W² represents a suitable group such as an appropriate alkali metal group (e.g. sodium, potassium or, especially, lithium), a —Mg-halide or a zinc-based group, and ring A, D₁, D₂ₐ, D_{2b}, D₃, Z^{q2} and T^{x} are as hereinbefore defined, with e.g. CO₂ (in the case where R^{9b} in the compounds to be prepared represents hydrogen) or a compound of formula XIV in which L^{xy} represents a single bond, Y^{b} represents —C(O)OR^{9a}, in which R^{9a} is other than hydrogen, and L⁶ represents a suitable leaving group, such as chloro or bromo or a C₁₋₁₄ (such as C₁₋₆ (e.g. C₁₋₃) alkoxy group), under reaction conditions known to those skilled in the art. The skilled person will appreciate that this reaction step may be performed directly after (i.e. in the same reaction pot) the preparation of compounds of formula XXXI (which is described hereinafter).

Compounds of formula IX in which Z^{x} represents a sulfonate group may be prepared from corresponding compounds in which the Z^{x} group represents a hydroxy group, with an appropriate reagent for the conversion of the hydroxy group to the sulfonate group (e.g. tosyl chloride, mesyl chloride, triflic anhydride and the like) under conditions known to those skilled in the art, for example in the presence of a suitable base and solvent (such as those described above in respect of process step (i), e.g. an aqueous solution of K₃PO₄ in toluene) preferably at or below room temperature (e.g. at about 10° C.).

Compounds of formulae XX and XXI may be prepared, for example, by reaction of a corresponding compound of formula XXIII or XXII, respectively (all of which are as hereinbefore defined, e.g. in which L^{5b} represents bromo or, preferably, iodo), for example, in the presence of a nucleophile that is a source of cyano ions, e.g. potassium or, preferably, copper cyanide.

Compounds of formulae XXII and XXIII in which L^{5b} represents a —Mg-halide may be prepared by reaction of a compound corresponding to a compound of formula XXII or XXIII but in which L^{5b} represents a halo group (e.g. bromo or iodo), under standard Grignard formation conditions, for example in the presence of i-PrMgCl (or the like) in the presence of a polar aprotic solvent (such as THF) under inert reaction condition, and preferably at low temperature (such as at below 0° C., e.g. at about 30° C.). The skilled person will appreciate that these compounds may be prepared in situ (see e.g. the process for the preparation of compounds of formula I (process steps (xvi) and (xvii)).

Compounds of formulae XXIIIC or XXIIID may be prepared by reaction of a corresponding compound of formula XXIII or XXII, as hereinbefore defined (and preferably one in which L^{5b} is a —Mg-halide, such as —Mg—I), with dimethylformamide (or a similar reagent for the introduction of the aldehyde group), under standard Grignard reaction conditions known to those skilled in the art (for example those described herein).

Compounds of formulae XXIX or XXX in which T^{x} represents —CH₂— may be prepared by reduction of a corresponding compound of formulae XXIX or XXX in which T^{x} represents —C(O)— (or from compounds corresponding to compounds of formulae XXIX or XXX but in which T^{x} represents —CH(OH)—), for example under standard reaction conditions known to those skilled in the art, for example reduction in the presence of a suitable reducing reagent such as LiAlH₄, NaBH₄ or trialkylsilane (e.g. triethylsilane) or reduction by hydrogenation (e.g. in the presence of Pd/C).

Alternatively, compounds of formulae XXIX or XXX in which T^{x} represents —CH₂— may be prepared by reaction of a compound of formula XXXII,

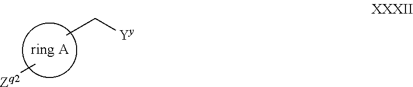

wherein Y^{y} represents a suitable group such as —OH, bromo, chloro or iodo, and ring A and Z^{q2} are as hereinbefore defined, with a compound of formula XXXIII,

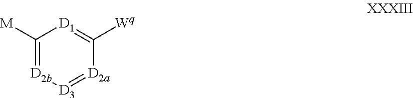

wherein M represents hydrogen and W^{q} represents hydrogen (for compounds of formula XXIX) or W¹ (for compounds of formula XXX) and D₁, D₂ₐ, D_{2b} and D₃ are as hereinbefore defined, under standard conditions, for example in the presence of a Lewis or Brønsted acid. Alternatively, such compounds may be prepared from reaction of a compound of formula XXXII in which Y represents bromo or chloro with a compound corresponding to a compound of formula XXXIII but in which M represents —BF₃K (or the like), for example in accordance with the procedures described in Molander et al, J. Org. Chem. 71, 9198 (2006).

Compounds of formulae XXIX or XXX in which T^{x} represents —C(O)— may be prepared by reaction of a compound of formula XXXIV,

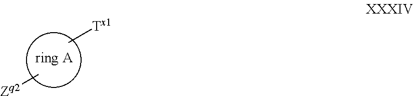

wherein T^{x1} represents —C(O)Cl or —C═N—NH(t-butyl) (or the like) and ring A and Z^{q2} are as hereinbefore defined, with a compound of formula XXXIII in which M represents hydrogen or an appropriate alkali metal group (e.g. sodium, potassium or, especially, lithium), a —Mg-halide or a zinc-based group, or, a bromo group, and D₁, D₂ₐ, D_{2b}, D₃ and W^{q} are as hereinbefore defined, under reaction conditions known to those skilled in the art. For example in the case of reaction of a compound of formula XXXIV in which T^{x1} represents —C(O)Cl with a compound of formula XXXIII in which M represents hydrogen, in the presence of an appropriate Lewis acid. In the case where M represents an appropriate alkali metal group, a —Mg-halide or a zinc-based group, under reaction conditions such as those hereinbefore described in respect of preparation of compounds of formulae III, IX, XXIV or XXVI (process step (IV) above) and preparation of compounds of formula XXXI (see below). In the case of a reaction of a compound of formula XXXIV in which T^{x1} represents —C═N—NH(t-butyl) (or the like) with a compound of formula XXXIII in which M represents bromo, under reaction conditions such as those described in Takemiya et al, J. Am. Chem. Soc. 128, 14800 (2006).

For compounds corresponding to compounds of formula XXIX or XXX in which TX represents —CH(OH)—, reaction of a compound corresponding to a compound of formula XXXIV, but in which T^{x1} represents —C(O)H, with a compound of formula XXXIII as defined above, under reaction conditions such as those hereinbefore described in respect of preparation of compounds of formulae XXIX or XXX in which T^{x} represents —C(O)—.

Compounds of formula XXXI may be prepared in several ways. For example, compounds of formula XXXI in which W² represents an alkali metal such as lithium, may be prepared from a corresponding compound of formula XXIX (in particular those in which Z^{q2} represents a chloro or sulfonate group or, especially, a protected —NH₂ group, wherein the protecting group is preferably a lithiation-directing group, e.g. an amido group, such as a pivaloylamido group, or a sulfonamido group, such as an arylsulfonamido group, e.g. phenylsulfonamide), by reaction with an organolithium base, such as n-BuLi, s-BuLi, t-BuLi, lithium diisopropylamide or lithium 2,2,6,6-tetramethylpiperidine (which organolithium base is optionally in the presence of an additive (for example, a lithium co-ordinating agent such as an ether (e.g. dimethoxyethane) or an amine (e.g. tetramethylethylenediamine (TMEDA), (−)sparteine or 1,3-dimethyl-3,4,5,6-tetrahydro-2(1H)-pyrimidinone (DMPU) and the like)), for example in the presence of a suitable solvent, such as a polar aprotic solvent (e.g. tetrahydrofuran or diethyl ether), at sub-ambient temperatures (e.g. 0° C. to −78° C.) under an inert atmosphere. Alternatively, such compounds of formula XXXI may be prepared by reaction of a compound of formula XXX in which W¹ represents chloro, bromo or iodo by a halogen-lithium reaction in the presence of an organolithium base such as t- or n-butyllithium under reaction conditions such as those described above. Compounds of formula XXXI in which W² represents —Mg-halide may be prepared from a corresponding compound of formula XXX in which W¹ represents halo (e.g. bromo), for example optionally in the presence of a catalyst (e.g. FeCl₃) under standard Grignard conditions known to those skilled in the art. The skilled person will also appreciate that the magnesium of the Grignard reagent or the lithium of the lithiated species may be exchanged to a different metal (i.e. a transmetallation reaction may be performed), for example to form compounds of formula XXXI in which W² represents a zinc-based group (e.g. using ZnCl₂).

Compounds mentioned herein (e.g. those of formulae IV, V, VA, VI, VII, X, XA, XI, XII, XIII, XIV, XIVa, XIVb, XV, XVI, XVII, XVIII, XIX, XX, XXI, XXII, XXIII, XXIIIA, XXIIIB, XXV, XXVII, XXVIII, XXXII, XXXIII and XXXIV) are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further, the compounds described herein may also be prepared in accordance with synthetic routes and techniques described in international patent application WO 2006/077366.

The substituents D₁, D₂ₐ, D_{2b}, D₃, L¹, Y¹, L² and Y² in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, etherifications, halogenations or nitrations. Such reactions may result in the formation of a symmetric or asymmetric final compound of the invention or intermediate. The precursor groups can be changed to a different such group, or to the groups defined in formula I, at any time during the reaction sequence. For example, in cases where Y¹ (or, if present, Y^{1a}) represents —C(O)OR^{9a} in which R^{9a} does not initially represent hydrogen (so providing at least one ester functional group), the skilled person will appreciate that at any stage during the synthesis (e.g. the final step), the relevant R^{9b}-containing group may be hydrolysed to form a carboxylic acid functional group (i.e. a group in which R^{9b} represents hydrogen). In this respect, the skilled person may also refer to “Comprehensive Organic Functional Group Transformations” by A. R. Katritzky, O. Meth-Cohn and C. W. Rees, Pergamon Press, 1995. Other specific transformation steps include the reduction of a nitro group to an amino group, the hydrolysis of a nitrile group to a carboxylic acid group, and standard nucleophilic aromatic substitution reactions, for example in which an iodo-, preferably, fluoro- or bromo-phenyl group is converted into a cyanophenyl group by employing a source of cyanide ions (e.g. by reaction with a compound which is a source of cyano anions, e.g. sodium, copper (I), zinc or, preferably, potassium cyanide) as a reagent (alternatively, in this case, palladium catalysed cyanation reaction conditions may also be employed).

Other transformations that may be mentioned include: the conversion of a halo group (preferably iodo or bromo) to a 1-alkynyl group (e.g. by reaction with a 1-alkyne), which latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine); the introduction of amino groups and hydroxy groups in accordance with standard conditions using reagents known to those skilled in the art; the conversion of an amino group to a halo, azido or a cyano group, for example via diazotisation (e.g. generated in situ by reaction with NaNO₂ and a strong acid, such as HCl or H₂SO₄, at low temperature such as at 0° C. or below, e.g. at about −5° C.) followed by reaction with the appropriate reagent/nucleophile e.g. a source of the relevant reagent/anion, for example by reaction in the presence of a reagent that is a source of halogen (e.g. CuCl, CuBr or NaI), or a reagent that is a source of azido or cyanide anions, such as NaN₃, CuCN or NaCN; the conversion of —C(O)OH to a —NH₂ group, under Schmidt reaction conditions, or variants thereof, for example in the presence of HN₃ (which may be formed in by contacting NaN₃ with a strong acid such as H₂SO₄), or, for variants, by reaction with diphenyl phosphoryl azide ((PhO)₂P(O)N₃) in the presence of an alcohol, such as tert-butanol, which may result in the formation of a carbamate intermediate; the conversion of —C(O)NH₂ to —NH₂, for example under Hofmann rearrangement reaction conditions, for example in the presence of NaOBr (which may be formed by contacting NaOH and Br₂) which may result in the formation of a carbamate intermediate; the conversion of —C(O)N₃ (which compound itself may be prepared from the corresponding acyl hydrazide under standard diazotisation reaction conditions, e.g. in the presence of NaNO₂ and a strong acid such as H₂SO₄ or HCl) to —NH₂, for example under Curtius rearrangement reaction conditions, which may result in the formation of an intermediate isocyanate (or a carbamate if treated with an alcohol); the conversion of an alkyl carbamate to —NH₂, by hydrolysis, for example in the presence of water and base or under acidic conditions, or, when a benzyl carbamate intermediate is formed, under hydrogenation reaction conditions (e.g. catalytic hydrogenation reaction conditions in the presence of a precious metal catalyst such as Pd); halogenation of an aromatic ring, for example by an electrophilic aromatic substitution reaction in the presence of halogen atoms (e.g. chlorine, bromine, etc, or an equivalent source thereof) and, if necessary an appropriate catalyst/Lewis acid (e.g. AlCl₃ or FeCl₃).

Further, the skilled person will appreciate that the D₁ to D₃-containing ring, as well as the A ring may be heterocycles, which moieties may be prepared with reference to a standard heterocyclic chemistry textbook (e.g. “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 or “Science of Synthesis”, Volumes 9-17 (Hetarenes and Related Ring Systems), Georg Thieme Verlag, 2006). Hence, the reactions disclosed herein that relate to compounds containing hetereocycles may also be performed with compounds that are pre-cursors to heterocycles, and which pre-cursors may be converted to those heterocycles at a later stage in the synthesis.

Compounds of the invention may be isolated (or purified) from their reaction mixtures using conventional techniques (e.g. crystallisations, recrystallisations or chromatographic techniques).

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques. By ‘protecting group’ we also include suitable alternative groups that are precursors to the actual group that it is desired to protect. For example, instead of a ‘standard’ amino protecting group, a nitro or azido group may be employed to effectively serve as an amino protecting group, which groups may be later converted (having served the purpose of acting as a protecting group) to the amino group, for example under standard reduction conditions described herein. Protecting groups that may be mentioned include lactone protecting groups (or derivatives thereof), which may serve to protect both a hydroxy group and an α-carboxy group (i.e. such that the cyclic moiety is formed between the two functional groups.

The type of chemistry involved will dictate the need, and type, of protecting groups as well as the sequence for accomplishing the synthesis.

The use of protecting groups is described in e.g. “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined, for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention, including, but not limited to:
(a) compounds of formula I in which Y¹ (or, if present, Y^{1a}) represents —C(O)OR^{9a} in which R^{9a} is/are other than hydrogen, so forming an ester group; and/or
(b) compounds of formula I in which Y represents —C(═N—OR²⁹)—, i.e. the following compound of formula Ia,

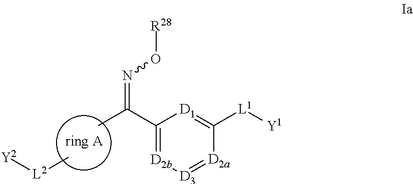

in which the integers are as hereinbefore defined (and the squiggly line indicates that the oxime may exist as a cis or trans isomer, as is apparent to the skilled person),
   may possess no or minimal pharmacological activity as such, but may be administered parenterally or orally, and thereafter be metabolised in the body to form compounds of the invention that possess pharmacological activity as such, including, but not limited to:
(A) corresponding compounds of formula I, in which Y¹ (or, if present, Y^{1a}) represents —C(O)OR^{9a} in which R^{9a} represent hydrogen (see (a) above); and/or
(B) corresponding compounds of formula I in which Y represents —C(O)—, for example in the case where the oxime or oxime ether of the compound of formula Ia (see (b) above) is hydrolysed to the corresponding carbonyl moiety.

Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention may inhibit leukotriene (LT) C₄ synthase, for example as may be shown in the test described below, and may thus be useful in the treatment of those conditions in which it is required that the formation of e.g. LTC₄, LTD₄ or LTE₄ is inhibited or decreased, or where it is required that the activation of a Cys-LT receptor (e.g. Cys-LT₁ or Cys-LT₂) is inhibited or attenuated. The compounds of the invention may also inhibit microsomal glutathione S-transferases (MGSTs), such as MGST-I, MGST-II and/or MGST-III (preferably, MGST-II), thereby inhibiting or decreasing the formation of LTD₄, LTE₄ or, especially, LTC₄.

Compounds of the invention may also inhibit the activity of 5-lipoxygenase-activating protein (FLAP), for example as may be shown in a test such as that described in Mol. Pharmacol., 41, 873-879 (1992). Hence, compounds of the invention may also be useful in inhibiting or decreasing the formation of LTC₄ and/or LTB₄.

Compounds of the invention are thus expected to be useful in the treatment of disorders that may benefit from inhibition of production (i.e. synthesis and/or biosynthesis) of leukotrienes (such as LTC₄), for example a respiratory disorder and/or inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compounds of the invention may be useful in the treatment of allergic disorders, asthma, childhood wheezing, chronic obstructive pulmonary disease, bronchopulmonary dysplasia, cystic fibrosis, interstitial lung disease (e.g. sarcoidosis, pulmonary fibrosis, scleroderma lung disease, and usual interstitial in pneumonia), ear nose and throat diseases (e.g. rhinitis, nasal polyposis, and otitis media), eye diseases (e.g. conjunctivitis and giant papillary conjunctivitis), skin diseases (e.g. psoriasis, dermatitis, and eczema), rheumatic diseases (e.g. rheumatoid arthritis, arthrosis, psoriasis arthritis, osteoarthritis, systemic lupus erythematosus, systemic sclerosis), vasculitis (e.g. Henoch-Schonlein purpura, Löffler's syndrome and Kawasaki disease), cardiovascular diseases (e.g. atherosclerosis), gastrointestinal diseases (e.g. eosinophilic diseases in the gastrointestinal system, inflammatory bowel disease, irritable bowel syndrome, colitis, celiaci and gastric haemorrhagia), urologic diseases (e.g. glomerulonephritis, interstitial cystitis, nephritis, nephropathy, nephrotic syndrome, hepatorenal syndrome, and nephrotoxicity), diseases of the central nervous system (e.g. cerebral ischemia, spinal cord injury, migraine, multiple sclerosis, and sleep-disordered breathing), endocrine diseases (e.g. autoimmune thyreoiditis, diabetes-related inflammation), urticaria, anaphylaxis, angioedema, oedema in Kwashiorkor, dysmenorrhoea, burn-induced oxidative injury, multiple trauma, pain, toxic oil syndrome, endotoxin chock, sepsis, bacterial infections (e.g. from Helicobacter pylori, Pseudomonas aerugiosa or Shigella dysenteriae), fungal infections (e.g. vulvovaginal candidasis), viral infections (e.g. hepatitis, meningitis, parainfluenza and respiratory syncytial virus), sickle cell anemia, hypereosinofilic syndrome, and malignancies (e.g. Hodgkins lymphoma, leukemia (e.g. eosinophil leukemia and chronic myelogenous leukemia), mastocytos, polycytemi vera, and ovarian carcinoma). In particular, compounds of the invention may be useful in treating allergic disorders, asthma, rhinitis, conjunctivitis, COPD, cystic fibrosis, dermatitis, urticaria, eosinophilic gastrointestinal diseases, inflammatory bowel disease, rheumatoid arthritis, osteoarthritis and pain.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, LTC₄ synthase and/or a method of treatment of a disease in which inhibition of the synthesis of LTC₄ is desired and/or required (e.g. respiratory disorders and/or inflammation), which method comprises administration of a therapeutically effective amount of a compound of the invention, as hereinbefore defined, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined, in admixture with a pharmaceutically acceptable adjuvant, diluent or carrier.

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The invention further provides a process for the preparation of a pharmaceutical formulation, as hereinbefore defined, which process comprises bringing into association a compound of the invention, as hereinbefore defined, or a pharmaceutically acceptable salt thereof with a pharmaceutically-acceptable adjuvant, diluent or carrier.

Compounds of the invention may also be combined with other therapeutic agents that are useful in the treatment of a respiratory disorder (e.g. leukotriene receptor antagonists (LTRas), glucocorticoids, antihistamines, beta-adrenergic drugs, anticholinergic drugs and PDE₄ inhibitors and/or other therapeutic agents that are useful in the treatment of a respiratory disorder) and/or other therapeutic agents that are useful in the treatment of inflammation and disorders with an inflammatory component (e.g. NSAIDs, coxibs, corticosteroids, analgesics, inhibitors of 5-lipoxygenase, inhibitors of FLAP (5-lipoxygenase activating protein), immunosuppressants and sulphasalazine and related compounds and/or other therapeutic agents that are useful in the treatment of inflammation).

According to a further aspect of the invention, there is provided a combination product comprising:
(A) a compound of the invention, as hereinbefore defined; and
(B) another therapeutic agent that is useful in the treatment of a respiratory disorder and/or inflammation,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined, another therapeutic agent that is useful in the treatment of a respiratory disorder and/or inflammation, and a pharmaceutically-acceptable adjuvant, diluent or carrier; and
(2) a kit of parts comprising components:
(a) a pharmaceutical formulation including a compound of the invention, as hereinbefore defined, in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of the invention, as hereinbefore defined, or a pharmaceutically acceptable salt thereof with the other therapeutic agent that is useful in the treatment of a respiratory disorder and/or inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Aqueous solubility is a fundamental molecular property that governs a large range of physical phenomena related to the specific chemical compound including e.g. environmental fate, human intestinal absorption, effectiveness of in vitro screening assays, and product qualities of water-soluble chemicals. By definition, the solubility of a compound is the maximum quantity of compound that can dissolve in a certain quantity of solvent at a specified temperature. Knowledge of a compound's aqueous solubility can lead to an understanding of its pharmacokinetics, as well as an appropriate means of formulation.

Compounds of the invention may exhibit improved solubility properties. Greater aqueous solubility (or greater aqueous thermodynamic solubility) may have advantages related to the effectiveness of the compounds of the invention, for instance improved absorption in vivo (e.g. in the human intestine) or the compounds may have other advantages associated with the physical phenomena related to improved aqueous stability (see above). Good (e.g. improved) aqueous solubility may aid the formulation of compounds of the invention, i.e. it may be easier and/or less expensive to manufacture tablets which will dissolve more readily in the stomach as potentially one can avoid esoteric and/or expensive additives and be less dependent on particle-size (e.g. micronization or grinding may be avoided) of the crystals, etc, and it may be easier to prepare formulations intended for intravenous administration.

Compounds of the invention may have the advantage that they are effective inhibitors of LTC₄ synthase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### Biological Test

### In Vitro Assay

In the assay, LTC₄ synthase catalyses the reaction where the substrate LTA₄ is converted to LTC₄. Recombinant human LTC₄ synthase is expressed in Piccia pastoralis and the purified enzyme is dissolved in 25 mM tris-buffer pH 7.8 supplemented with 0.1 mM glutathione (GSH) and stored at −80° C. The assay is performed in phosphate buffered saline (PBS) pH 7.4 and 5 mM GSH in 384-well plates.

The following is added chronologically to each well:

1. 48 μL LTC₄ synthase in PBS with 5 mM GSH. The total protein concentration in this solution is 0.5 μg/mL.
2. 1 μL inhibitor in DMSO (final concentration 10 μM).
3. Incubation of the plate at room temperature for 10 min.
4. 1 μL LTA₄ (final concentration 2.5 μM).
5. Incubation of the plate at room temperature for 5 min.
6. 10 μL of the incubation mixture is analysed using homogenous time resolved fluorescent (HTRF) detection.

### EXAMPLES

In the event that there is a discrepancy between nomenclature and any compounds depicted graphically, then it is the latter that presides (unless contradicted by any experimental details that may be given or unless it is clear from the context).

The invention is illustrated by way of the following examples, in which the following abbreviations may be employed:

aq aqueous
BINAP 2,2′-bis(diphenylphosphino)-1,1′-binaphthyl
brine saturated aqueous solution of NaCl
Cp*RuClCOD chloro(pentamethylcyclopentadienyl)(cyclooctadiene)ruthenium(II)
DMF dimethylformamide
DMSO dimethylsulfoxide
EtOAc ethyl acetate
EtOH ethanol
MeCN acetonitrile
MeOH methanol
NMR nuclear magnetic resonance
rt room temperature
Tx reflux
sat saturated
THF tetrahydrofuran

### Example 1

### 5-{4-[(4-Chlorophenyl)(methyl)amino]benzoyl}-2-(5,6-dimethyl-1-benzimidazolyl)-benzoic acid

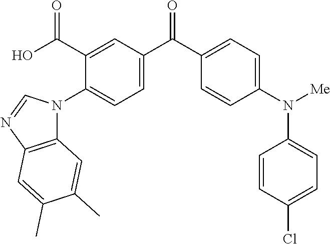

### (a) 5-(4-Bromobenzoyl)-2-fluorobenzoic acid methyl ester

i-PrMgCl.LiCl in THF (0.78 M, 33.5 mL, 26.2 mmol) was added to 2-fluoro-5-iodo-benzoic acid methyl ester (5.23 g, 18.7 mmol) in THF (20 mL) at −30° C. After 2 h at that temperature, the mixture was cooled to −65° C. and 4-bromobenzoyl chloride (9.02 g, 41.1 mmol) in THF (25 mL) was added. The mixture was stirred at −40° C. for 4 h, the temperature was allowed to reach rt and NH₄Cl (aq, sat) was added. Extractive workup (EtOAc, K₂CO₃ (aq, sat), H₂O, brine), concentration and purification by chromatography gave the sub-title compound. Yield: 3.43 g (53%).

### (b) 5-{4-[(4-Chlorophenyl)amino]benzoyl}-2-fluorobenzoic acid methyl ester

A mixture of 5-(4-bromobenzoyl)-2-fluorobenzoic acid methyl ester (3.27 g, 9.70 mmol), Pd(OAc)₂ (109 mg, 0.48 mmol), BINAP (453 mg, 0.73 mmol), Cs₂CO₃ (4.42 g, 13.6 mmol) and toluene (35 mL) was stirred at rt for 10 min. 4-Chloro-N-methylaniline (1.42 mL, 11.64 mmol) was added and the mixture was heated at 110° C. for 20 h. The mixture was diluted with EtOAc and filtered through Celite. Concentration and purification by chromatography gave the sub-title compound. Yield: 2.22 g (57%).

### (c) 5-{4-[(4-Chlorophenyl)(methyl)amino]benzoyl}-2-(5,6-dimethyl-1-benzimidazolyl)benzoic acid methyl ester

A mixture of 5-{5-[(4-chlorophenyl)amino]benzoyl}-2-fluorobenzoic acid methyl ester (0.15 g, 0.38 mmol), 5,6-dimethylbenzimidazole (56 mg, 0.38 mmol), KF/Al₂O₃ (138 mg), 18-crown-6 (10 mg, 0.04 mmol) and MeCN (3 mL) was heated at rx for 18 h. Extractive workup (EtOAc, HCl (1 M), H₂O, brine), drying (Na₂SO₄), concentration and purification by chromatography gave the sub-title compound. Yield: 144 mg (72%).

### (d) 5-{4-[(4-Chlorophenyl)(methyl)amino]benzoyl}-2-(5,6-dimethyl-1-benzimidazolyl)benzoic acid

A mixture of 5-{4-[(4-chlorophenyl)(methyl)amino]benzoyl}-2-(5,6-dimethyl-1-benzimidazolyl)benzoic acid methyl ester (139 mg, 0.27 mmol), NaOH (53 mg, 1.33 mmol), EtOH (4 mL) and H₂O (2 mL) was heated at 80° C. for 30 min. The pH was adjusted to ˜5 with HCl (1 M, aq). The precipitate was collected, washed with H₂O and recrystallised from EtOH/THF/H₂O to give the title compound.

Yield: 114 mg (83%).

¹H NMR (DMSO-d₆) δ: 13.4-13.1 (1H, br s) 8.25 (1H, s) 8.20 (1H, d, J=1.5 Hz) 8.00 (1H, dd, J=8.0, 1.5 Hz) 7.78-7.67 (3H, m) 7.56-7.44 (3H, m) 7.39-7.28 (2H, m) 7.06 (1H, s) 6.95-6.82 (2H, m) 3.35 (3H, s) 2.30 (3H, s) 2.26 (3H, s).

### Example 2:1

### 5-{4-[(4-Chlorophenyl)(methyl)amino]benzoyl}-2-(4-phenyl-1,2,3-triazol-1-yl)-benzoic acid

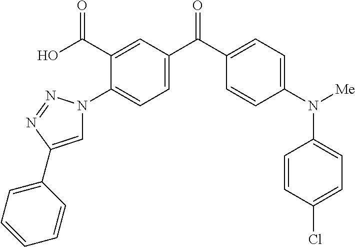

### (a) 2-Azido-5-{4-[(4-chlorophenyl)(methyl)amino]benzoyl}benzoic acid methyl ester

A mixture of 5-{4-[(4-chlorophenyl)amino]benzoyl}-2-fluorobenzoic acid methyl ester (8.13 g, 20.4 mmol, see Example 1, step (b)) and DMSO (20 mL) was added to NaN₃ (2.07 g, 41 mmol) in DMSO (250 mL) at 80° C. and stirred at that temperature for 3 h. The mixture was poured into ice-water and the precipitate was collected. Recrystallisation from EtOH gave the sub-title compound.

Yield: 8.14 g (94%).

### (b) 5-{4-[(4-Chlorophenyl)(methyl)amino]benzoyl}-2-(4-phenyl[1,2,3]triazol-1-yl)benzoic acid methyl ester

Ethynylbenzene (54 mg, 0.52 mmol) was added to 2-azido-5-{4-[(4-chlorophenyl)-(methyl)amino]benzoyl}benzoic acid methyl ester (0.21 g, 0.5 mmol) in dioxane (2.5 mL). A copper-complex prepared by stirring CuI (19 mg, 0.1 mmol), N,N′-di-methylethylenediamine (500 μL) and dioxane (5 mL) at 70° C. for 5 min and at rt for 3 h, was added. The mixture was stirred at rt for 2 h. Concentration and purification by chromatography gave the sub-title compound.

Yield: 161 mg (62%).

### (c) 5-{4-[(4-Chlorophenyl)(methyl)amino]benzoyl}-2-(4-phenyl-1,2,3-triazol-1-yl)benzoic acid

The title compound was prepared from 5-{4-[(4-chlorophenyl)(methyl)amino]-benzoyl}-2-(4-phenyl[1,2,3]triazol-1-yl)benzoic acid methyl ester (161 mg) by hydrolysis in accordance with Example 1, step (d). Yield: 115 mg (73%).

¹H NMR (DMSO-d₆) δ: 13.5-13.4 (1H, br s) 9.10 (1H, s) 8.13 (1H, d, J=1.8 Hz) 8.03 (1H, dd, J=8.2, 1.8 Hz) 7.98-7.92 (2H, m) 7.87 (1H, d, J=8.2 Hz) 7.76-7.68 (2H, m) 7.55-7.45 (4H, m) 7.42-7.30 (3H, m) 6.95-6.86 (2H, m) 3.37 (3H, s).

### Example 2:2

### 5-{4-[(4-Chlorophenyl)(methyl)amino]benzoyl}-2-[4-(3-chlorophenyl)-1,2,3-triazol-1-yl]benzoic acid

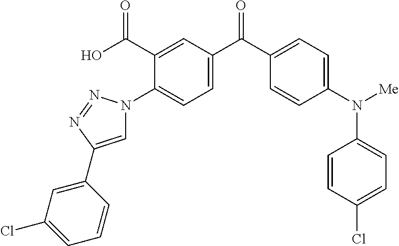

The title compound was prepared from 2-azido-5-{4-[(4-chlorophenyl)(methyl)-amino]benzoyl}benzoic acid methyl ester and 1-chloro-3-ethynylbenzene in accordance with Example 2:1.

¹H NMR (DMSO-d₆) δ: 13.51 (1H, s) 9.21 (1H, s) 8.14 (1H, d, J=1.8 Hz) 8.06-7.98 (2H, m) 7.96-7.90 (1H, m) 7.87 (1H, d, J=8.2 Hz) 7.76-7.68 (2H, m) 7.58-7.49 (3H, m) 7.48-7.42 (1H, m) 7.38-7.30 (2H, m) 6.94-6.86 (2H, m) 3.37 (3H, s).

### Example 3:1

### 5-{4-[(4-Chlorophenyl)(methyl)amino]benzoyl}-2-(5-phenyl-1,2,3-triazol-1-yl)-benzoic acid

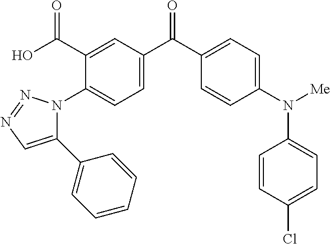

### (a) 5-{4-[(4-Chlorophenyl)(methyl)amino]benzoyl}-2-(5-phenyl-1,2,3-triazol-1-yl)benzoic acid methyl ester

Cp*RuClCOD (83.1 mg, 0.22 mmol) was added to a mixture of 2-azido-5-{4-[(4-chlorophenyl)(methyl)amino]benzoyl}benzoic acid methyl ester (463 mg, 1.1 mmol, see Example 2:1, step (a)), 1-ethynylbenzene (102 mg, 1 mmol) and DMF (6 mL). The mixture was heated at 110° C. for 20 min in a sealed vessel using microwave irradiation. Extractive workup (EtOAc, H₂O, brine), drying (Na₂SO₄), concentration and purification by chromatography gave the sub-title compound. Yield: 138 mg (26%).

### (b) 5-{4-[(4-Chlorophenyl)(methyl)amino]benzoyl}-2-(5-phenyl-1,2,3-triazol-1-yl)benzoic acid

The title compound was prepared from 5-{4-[(4-chlorophenyl)(methyl)amino]-benzoyl}-2-(5-phenyl[1,2,3]triazol-1-yl)benzoic acid methyl ester (138 mg) by hydrolysis in accordance with Example 1, step (d). Yield: 67 mg (50%).

¹H NMR (DMSO-d₆) δ: 13.40 (1H, s) 8.17-8.11 (2H, m) 7.94 (1H, dd, J=8.2, 1.8 Hz) 7.74-7.64 (2H, m) 7.60 (1H, d, J=8.2 Hz) 7.54-7.47 (2H, m) 7.44-7.25 (7H, m) 6.92-6.85 (2H, m) 3.36 (3H, s)

### Example 3:2

5-{4-[(4-Chlorophenyl)(methyl)amino]benzoyl}-2-[5-(3-chlorophenyl)-1,2,3-triazol-1-yl]benzoic acid

The title compound was prepared from 2-azido-5-{4-[(4-chlorophenyl)(methyl)-amino]benzoyl}benzoic acid methyl ester and 1-chloro-3-ethynylbenzene in accordance with Example 3:1.

¹H NMR (DMSO-d₆) δ: 13.48 (1H, s) 8.25 (1H, s) 8.14 (1H, d, J=1.8 Hz) 7.96 (1H, dd, J=8.1, 1.8 Hz) 7.74-7.64 (3H, m) 7.54-7.30 (7H, m) 7.18-7.13 (1H, m) 6.92-6.84 (2H, m) 3.36 (3H, s).

### Example 4:1

### 5-{5-[(4-Chlorophenyl)(methyl)amino]picolinoyl}-2-(5-phenyl-1,2,3-triazol-1-yl)-benzoic acid

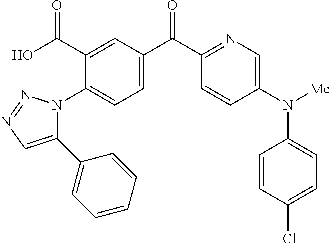

### (a) 2-Fluoro-5-formylbenzoic acid methyl ester

i-PrMgCl.LiCl complex in THF (1.0 M, 70 mL, 70.0 mmol) was added to 2-fluoro-5-iodobenzoic acid methyl ester (13.0 g, 46.4 mmol) in THF (80 mL) at −45° C. After stirring at −40° C. for 1 h, DMF (2.7 mL, 35.7 mmol) was added. The temperature was allowed to reach rt over 1 h and HCl (1 M, aq, 300 mL) was added. Extractive workup (EtOAc, water, brine) and concentration gave the sub-title product. Yield: 8.95 g (98%).

### (b) 5-[(5-Bromo-2-pyridyl)hydroxymethyl]-2-fluorobenzoic acid methyl ester

i-PrMgCl in THF (2.0M, 24 mL, 48.9 mmol) was added to 5-bromo-2-iodopyridine (13.2 g, 46.6 mmol) in THF (50 mL) at −15° C. After stirring at −15° C. for 1 h, 2-fluoro-5-formylbenzoic acid methyl ester (8.50 g, 48.9 mmol) in THF (50 mL) was added at −45° C. The mixture was stirred at rt for 6 h and quenched with NH₄Cl (aq, sat). Extractive workup (EtOAc, water, brine) and purification by chromatography gave the sub-title compound. Yield: 13.4 g (85%).

### (c) 5-(5-Bromopyridine-2-carbonyl)-2-fluoro-benzoic acid methyl ester

Pyridinium chlorochromate (8.94 g, 41.5 mmol) was added to 5-[(5-bromo-2-pyridinyl)hydroxymethyl]-2-fluorobenzoic acid methyl ester (13.4 g, 39.5 mmol) in CH₂Cl₂ (400 mL) at rt. After 1 h the mixture was filtered though Celite and concentrated. The residue was treated with EtOAc and hexane (1:2) and filtered through silica gel. Concentration of the combined filtrates gave the sub-title compound. Yield: 10.7 g (80%).

### (d) 5-{5-[(4-Chlorophenyl)methylamino]pyridine-2-carbonyl}-2-fluorobenzoic acid methyl ester

The sub-title compound was prepared from 5-(5-bromopyridine-2-carbonyl)-2-fluorobenzoic acid methyl ester and 4-chloro-N-methylaniline in accordance with Example 1, step (b).

### (e) 2-Azido-5-(5-((4-chlorophenyl)(methyl)amino)picolinoyl)benzoic acid methyl ester

5-{5-[(4-Chlorophenyl)methylamino]pyridine-2-carbonyl}-2-fluoro-benzoic acid methyl ester (5.45 g, 13.66 mmol) was added to NaN₃ (2.54 g, 39 mmol) in DMSO (200 mL). The mixture was stirred at 80° C. for 2 h, cooled to rt and pored into ice-water. The solid was collected and crystallised from EtOH to give the sub-title compound. Yield: 4.20 g (75%).

### (f) 5-{5-[(4-Chlorophenyl)(methyl)amino]picolinoyl}-2-(5-phenyl-1,2,3-triazol-1-yl)benzoic acid methyl ester

Cp*RuClCOD (76 mg, 0.20 mmol) was added to a mixture of 2-azido-5-(5-((4-chlorophenyl)(methyl)amino)picolinoyl)benzoic acid methyl ester (422 mg, 1.0 mmol), 1-ethynylbenzene (0.12 mL, 1.1 mmol) and DMF (6 mL). The mixture was heated at 110° C. for 20 min in a sealed vessel using microwave irradiation. Extractive workup (EtOAc, H₂O, brine), drying (Na₂SO₄), concentration and purification by chromatography gave the sub-title compound. Yield: 100 mg (19%).

### (g) 5-{5-[(4-Chlorophenyl)(methyl)amino]picolinoyl}-2-(5-phenyl[-2,3-triazol-1-yl)benzoic acid

The title compound was prepared from 5-{5-[(4-chlorophenyl)(methyl)amino]-picolinoyl}-2-(5-phenyl-1,2,3-triazol-1-yl)benzoic acid methyl ester by hydrolysis in accordance with Example 1, step (d). Yield: 93 mg (96%).

¹H NMR (DMSO-d₆) δ: 13.5-13.2 (1H, br s) 8.55 (1H, d, J=2.0 Hz) 8.28 (1H, dd, J=8.2, 1.9 Hz) 8.24 (1H, d, J=2.7 Hz) 8.16 (1H, s) 8.06 (1H, d, J=8.9 Hz) 7.59 (1H, d, J=8.2 Hz) 7.59-7.51 (2H, m) 7.47-7.36 (5H, m) 7.36-7.26 (3H, m) 3.43 (3H, s).

### Example 4:2

### 5-{4-[(4-Chlorophenyl)(methyl)amino]picolinoyl}-2-[5-(3-chlorophenyl)-1,2,3-triazol-1-yl]benzoic acid

The title compound was synthesized in accordance with example 4:1, using 1-chloro-4-ethynylbenzene in step (f), see Table 1.

### Example 4:3

### 5-{5-[(4-Chlorophenyl)(cyclopropylmethyl)amino]picolinoyl}-2-(5-phenyl[1,2,3]-triazol-1-yl)benzoic acid

### (a) 5-[5-(4-Chlorophenylamino)picolinoyl]-2-fluorobenzoic acid methyl ester

The sub-title compound was prepared from 5-(5-bromopyridine-2-carbonyl)-2-fluorobenzoic acid methyl ester (see example 4:1 step (c)) and 4-chloroaniline in accordance with Example 1, step (b).

### (b) 5-{5-[(4-Chlorophenyl)(cyclopropylmethylamino]picolinoyl}-2-fluorobenzoic acid methyl ester

NaH (60% in mineral oil, 0.329 g, 8.25 mmol) was added to a mixture of 5-[5-(4-chlorophenylamino)picolinoyl]-2-fluorobenzoic acid methyl ester (2.86 g, 7.71 mmol), bromomethylcyclopropane (3.12 g, 23.13 mmol) and DMF (58 mL) at 0° C. The mixture was stirred at rt for 5 h. Extractive workup (EtOAc, water, brine), concentration and purification by chromatography gave the sub-title compound. Yield: 2.32 g, 69%.

### (c) 5-{5-[(4-Chlorophenyl)(cyclopropylmethyl)amino]picolinoyl}-2-(5-phenyl-1,2,3-triazol-1-yl)benzoic acid

The title compound was synthesized from 5-{5-[(4-chlorophenyl)(cyclopropyl-methyl)amino]picolinoyl}-2-fluorobenzoic acid methyl ester and ethynylbenzene in accordance with Example 4:1 steps (e), (f) and (g), see Table 1.

### Examples 4:4-4:6

The title compounds were synthesized in accordance with example 4:3, using the appropriate alkyne in step (c), see Table 1.

TABLE 1

| | Chemical structure |
|---|---|
| Ex- | Name |
| ample | ¹H-NMR (DMSO-d₆, δ) |
| 4:2 | 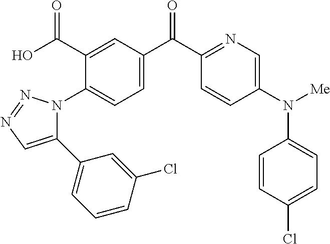 |
| | 5-{4-[(4-Chlorophenyl)(methyl)amino]picolinoyl}-2-[5- |
| | (3-chlorophenyl)-1,2,3-triazol-1-yl]benzoic acid |
| | 3.6-13.2 (1H, br s) 8.54 (1H, d, J = 1.8 Hz) 8.29 (1H, dd, J = 8.0, |
| | 1.6 Hz) 8.27-8.20 (2H, m) 8.07 (1H, d, J = 8.9) 7.64 (1H, d, J = |
| | 8.2 Hz) 7.60-7.52 (2H, m) 7.52-7.45 (2H, m) 7.45-7.36 (3H, m) 7.32 |
| | (1H, dd, J = 9.0, 2.8 Hz) 7.22-7.13 (1H, m) 3.43 (3H, s) |
| 4:3 | 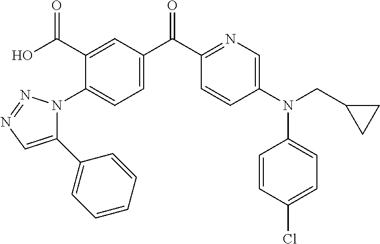 |
| | 5-{5-[(4-Chlorophenyl)(cyclopropylmethyl)amino]picolinoyl}-2- |
| | (5-phenyl-1,2,3-triazol-1-yl)benzoic acid |
| | 8.56-8.46 (1H, m) 8.26-8.14 (2H, m) 8.12 (1H, s) 8.03 (1H, d, J = |
| | 9.0 Hz) 7.63-7.52 (2H, m) 7.52-7.31 (8H, m) 7.28 (1H, dd, J = 9.0, |
| | 2.8 Hz) 3.74 (2H, d, J = 6.8 Hz) 1.17-1.04 (1H, m) 0.53-0.38 (2H, m) |
| | 0.24-0.08 (2H, m) |
| 4:4 | 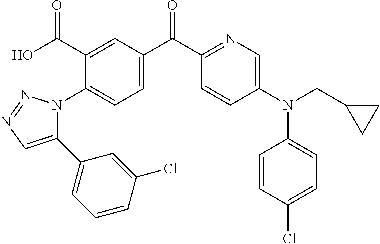 |
| | 5-{5-[(4-Chlorophenyl)(cyclopropylmethyl)amino]picolinoyl}-2- |
| | (5-(3-chlorophenyl)-1,2,3-triazol-1-yl)benzoic acid |
| | 8.49 (1H, s) 8.24-8.06 (3H, m) 8.03 (1H, d, J = 8.9 Hz) 7.63-7.49 |
| | (3H, m) 7.49-7.18 (7H, m) 3.74 (2H, d, J = 6.8 Hz) 1.16-1.06 |
| | (1H, m) 0.53-0.37 (2H, m) 0.24-0.09 (2H, m) |
| 4:5 | 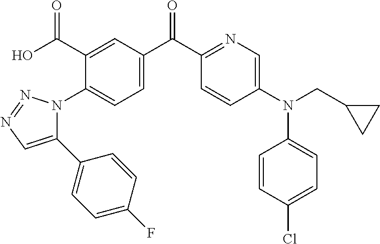 |
| | 5-{5-[(4-Chlorophenyl)(cyclopropylmethyl)amino]picolinoyl}-2- |
| | (5-(4-fluorophenyl)-1,2,3-triazol-1-yl)benzoic acid |
| | 8.52-8.44 (1H, m) 8.19-8.07 (3H, m) 8.07-7.97 (1H, d, J = 9.0 Hz) |
| | 7.62-7.51 (2H, m) 7.49-7.34 (5H, m) 7.33-7.17 (3H, m) 3.74 (2H, d, |
| | J = 6.4 Hz) 1.20-1.01 (1H, m) 0.52-0.39 (2H, m) 0.26-0.10 (2H, m) |
| 4:6 | 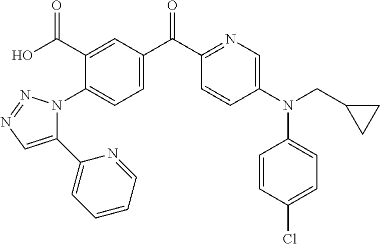 |
| | 5-{5-[(4-Chlorophenyl)(cyclopropylmethyl)amino]picolinoyl}-2- |
| | (5-(pyridin-2-yl)-1,2,3-triazol-1-yl)benzoic acid |
| | 8.55-8.47 (1H, m) 8.44 (1H, d, J = 4.1 Hz) 8.37 (1H, s) 8.21 (1H, |
| | s) 8.19 (1H, d, J = 2.8 Hz) 8.04 (1H, d, J = 9.0 Hz) 7.90-7.78 (1H, m) |
| | 7.68-7.61 (1H, m) 7.61-7.46 (3H, m) 7.46-7.37 (2H, m) 7.37-7.31 |
| | (1H, m) 7.29 (1H, dd, J = 9.0; 2.8 Hz) 3.75 (2H, d, J = 6.6 Hz) 1.17- |
| | 1.06 (1H, m) 0.54-0.39 (2H, m) 0.24-0.11 (2H, m) |

### Example 5:1

### 5-{5-[(4-Chlorophenyl)(cyclopropylmethyl)amino]picolinoyl}-2-(5-phenyl-3-trifluoro-methylpyrazol-1-yl)benzoic acid

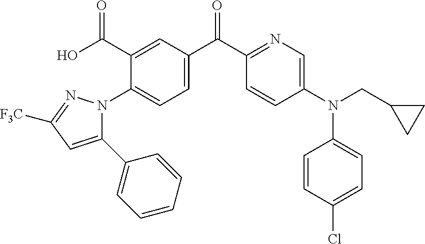

NaH (60% in mineral oil, 76 mg, 1.9 mmol) was added to 3-phenyl-5-trifluoromethyl-pyrazole (385 mg, 1.81 mmol) in DMSO (2 mL), and the mixture was stirred at rt for 20 min. 5-{5-[(4-Chlorophenyl)(cyclopropylmethyl)amino]picolinoyl}-2-fluorobenzoic acid methyl ester (700 mg, 1.65 mmol) (see Example 4:3, step (b)) in DMSO (5 mL) was added and the mixture was heated at 120° C. for 4 h. The mixture was poured into ice-water and extracted with EtOAc. The combined extracts were, washed with brine, dried (Na₂SO₄) and concentrated. Purification of the residue by chromatography and hydrolysis in accordance with Example 1, step (d)) gave the title compound.

¹H NMR (DMSO-d₆) δ: 13.8-13.0 (1H, br s) 8.40 (1H, s) 8.15 (1H, d, J=2.8 Hz) 8.1-8.0 (1H, br s) 7.99 (1H, d, J=9.0 Hz) 7.57-7.51 (2H, m) 7.41-7.30 (8H, m) 7.24 (1H, dd, J=9.0, 2.8 Hz) 7.13 (1H, s) 3.71 (2H, d, J=6.6 Hz) 1.12-1.05 (1H, m) 0.47-0.40 (2H, m) 0.18-0.12 (2H, m).

### Example 5:2

### 5-{5-[(4-Chlorophenyl)(methyl)amino]picolinoyl}-2-(3-phenyl-5-trifluoro-methylpyrazol-1-yl)benzoic acid

The title compound was prepared from 5-{5-[(4-chlorophenyl)methylamino]-pyridine-2-carbonyl}-2-fluorobenzoic acid methyl ester (see Example 4:1, step (d)) and 3-phenyl-5-trifluoromethylpyrazole in accordance with Example 5:1, see Table 2.

### Example 5:3

### 5-{5-[(4-Chlorophenyl)methylamino]pyridine-2-carbonyl}-2-(5-phenyl-3-trifluoro-methylpyrazol-1-yl)benzoic acid

5-{5-[(4-Chlorophenyl)methylamino]pyridine-2-carbonyl}-2-(5-phenyl-3-trifluoro-methylpyrazol-1-yl)benzoic acid methyl ester was isolated from the reaction of 5-{4-[(4-chlorophenyl)methylamino]pyridine-2-carbonyl}-2-fluorobenzoic acid methyl ester and 3-phenyl-5-trifluoromethylpyrazole (see Example 5:2). Hydrolysis in accordance with Example 1, step (d) gave the title compound, see Table 2.

### Example 5:4

### 5-{5-[(4-Chlorophenyl)(cyclopropylmethyl)amino]picolinoyl}-2-(3-phenyl-5-trifluoro-methylpyrazol-1-yl)benzoic acid

5-{5-[(4-Chlorophenyl)(cyclopropylmethyl)amino]picolinoyl}-2-(3-phenyl-5-trifluoro-methylpyrazol-1-yl)benzoic acid methyl ester was isolated from the reaction of 5-{5-[(4-chlorophenyl)(cyclopropylmethyl)amino]picolinoyl}-2-fluorobenzoic acid methyl ester and 3-phenyl-5-trifluoromethylpyrazole (see Example 5:1). Hydrolysis in accordance with Example 1, step (d) gave the title compound, see Table 2.

### Examples 5:5-5:7

The title compounds were prepared from 5-{5-[(4-chloro-phenyl)methylamino]-pyridine-2-carbonyl}-2-fluorobenzoic acid methyl ester (see Example 4:1, step (d)) and the appropriate pyrazole in accordance with Example 5:1, see Table 2.

TABLE 2

| | Chemical structure |
|---|---|
| Ex- | Name |
| ample | ¹H-NMR (DMSO-d₆, δ) |
| 5:2 | 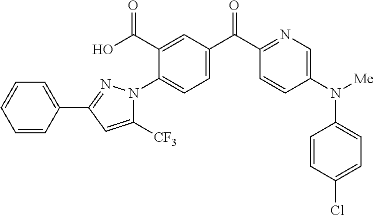 |
| | 5-{5-[(4-Chlorophenyl)(methyl)amino]picolinoyl}-2-(3-phenyl- |
| | 5-trifluoro-methylpyrazol-1-yl)benzoic acid |
| | 13.35-13.26 (1H, br s) 8.57 (1H, d, J = 2.0 Hz) 8.35 (1H, dd, J = 8.2, |
| | 2.0 Hz) 8.26 (1H, d, J = 2.8 Hz) 8.09 (1H, d, J = 8.8 Hz) 7.96-7.90 |
| | (2H, m) 7.82 (1H, d, J = 8.2 Hz) 7.73 (1H, s) 7.59-7.54 (2H, m) |
| | 7.52-7.39 (5H, m) 7.34 (1H, dd, J = 9.0, 3.2 Hz) 3.44 (3H, s) |
| 5:3 | 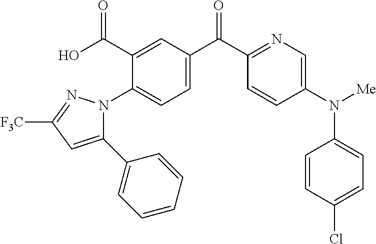 |
| | 5-{5-[(4-Chlorophenyl)methylamino]pyridine-2-carbonyl}-2- |
| | (5-phenyl-3-trifluoromethylpyrazol-1-yl)benzoic acid |
| | 13.40-13.28 (1H, br s) 8.49 (1H, d, J = 2.0 Hz) 8.25-8.20 (2H, m) |
| | 8.04 (1H, d, J = 9.0 Hz) 7.58-7.52 (3H, m) 7.43-7.29 (8H, m) 7.22 |
| | (1H, s) 3.43 (3H, s) |
| 5:4 | 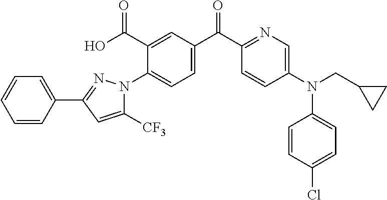 |
| | 5-{5-[(4-Chlorophenyl)(cyclopropylmethyl)amino]picolinoyl}-2- |
| | (3-phenyl-5-trifluoromethylpyrazol-1-yl)benzoic acid |
| | 13.4-13.2 (1H, br s) 8.56 (1 H, d, J = 1.8 Hz) 8.34 (1H, dd, J = 8.2, |
| | 1.8 Hz) 8.20 (1H, d, J = 2.8 Hz) 8.08 (1H, d, J = 9.0 Hz) 7.96- |
| | 7.90 (2H, m) 7.81 (1H, d, J = 8.2 Hz) 7.73 (1H, s) 7.61-7.55 (2H, m) |
| | 7.53-7.38 (5H, m) 7.30 (1H, dd, J = 9.0, 2.8 Hz) 3.76 (2H, d, J = |
| | 6.6 Hz) 1.15-1.08 (1H, m) 0.50-0.44 (2H, m) 0.22-0.15 (2H, m) |
| 5:5 | 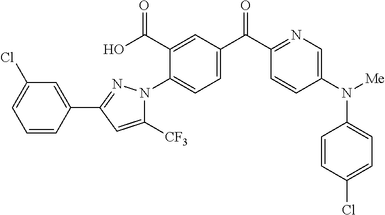 |
| | 5-{5-[(4-Chlorophenyl)(methyl)amino]picolinoyl}-2-[3-(3- |
| | chlorophenyl)-5-trifluoromethylpyrazol-1-yl]benzoic acid |
| | 8.90 (1H, d, J = 1.6 Hz) 8.46 (1H, d, J = 8.4 Hz) 8.25-8.19 (1H, m) |
| | 8.13 (1H, d, J = 8.4 Hz) 7.84 (1H, s) 7.70 (1H, d, J = 7 Hz) 7.63 (1H, |
| | d, J = 8.4 Hz) 7.44 (2H, d, J = 8.4 Hz) 7.37-7.33 (2H, m) 7.22 (2H, d, |
| | J = 8.4 Hz) 7.17 (1H, dd, J = 8.4, 1.6 Hz) 7.1 (1H, s) 3.44 (3H, s) |
| 5:6 | 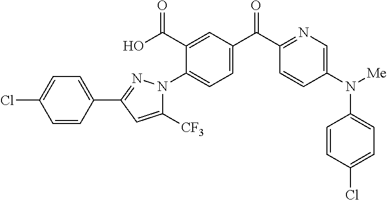 |
| | 5-{5-[(4-Chlorophenyl)(methyl)amino]picolinoyl}-2-[3-(4- |
| | chlorophenyl)-5-trifluoromethylpyrazol-1-yl]benzoic acid |
| | 8.74 (1H, d, J = 1.6 Hz) 8.35 (1H, dd, J = 8.2, 1.6 Hz) 8.11 (1H, d, J = |
| | 2.6 Hz) 8.01 (1H, d, J = 9.0 Hz) 7.67-7.61 (2H, m) 7.52 (1H, d, J = |
| | 8.2 Hz) 7.38-7.32 (2H, m) 7.31-7.26 (2H, m) 7.16-7.10 (2H, m) 7.07 |
| | (1H, dd, J = 8.8, 2.8 Hz) 6.95 (1H, s) 3.34 (3H, s) |
| 5:7 | 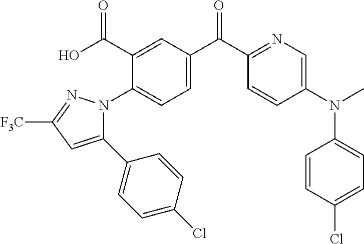 |
| | 5-{5-[(4-Chlorophenyl)(methyl)amino]picolinoyl}-2-[5-(4- |
| | chlorophenyl)-3-trifluoromethylpyrazol-1-yl]benzoic acid |
| | 8.80 (1H, d, J = 1.6 Hz) 8.35 (1H, dd, J = 8.4; 1.6 Hz ) 8.19 (1H, |
| | d, J = 1.6 Hz) 8.09 (1H, d, J = 8.6 Hz) 7.44-7.39 (2H, d, J = 8.6 Hz) |
| | 7.37 (1H, d, J = 8.6 Hz) 7.32-7.25 (2H, m, overlapped with CHCl₃) |
| | 7.21 (2H, d, J = 8.6 Hz) 7.32-7.25 (3H, m) 6.78 (1H, s) 3.44 (3H, s) |

### Example 6:1

### 5-{5-[(4-Chlorophenyl)(methyl)amino]picolinoyl}-2-(4-methyl-2-phenylimidazol-1-yl)benzoic acid

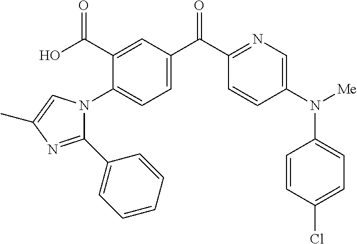

Sodium hydride (60% in mineral oil, 26 mg, 0.64 mmol) was added to 5-methyl-2-phenylimidazole (100 mg, 0.63 mmol) in DMSO (2 mL). The mixture was stirred at rt for 20 min and 5-{5-[(4-chlorophenyl)(methyl)amino]picolinoyl}-2-fluoro-benzoic acid methyl ester (0.25 g, 0.63 mmol, see Example 4:1, step (d) in DMSO (1.5 mL), was added. The mixture was heated at 130° C. for 5 d, poured into ice and extracted with EtOAc. The combined extracts were washed with brine, dried (Na₂SO₄) and concentrated. Purification by chromatography and hydrolysis in accordance with Example 1, step (d), gave the title compound.

¹H NMR (DMSO-d₆) δ: 8.53 (1H, d, J=2.0 Hz) 8.33-8.26 (2H, m) 8.09 (1H, d, J=9.0 Hz) 7.64-7.58 (2H, m) 7.54 (1H, J=8.2 Hz) 7.50-7.43 (2H, m) 7.40-7.30 (6H, m) 7.16 (1H, d, J=1.0 Hz) 3.48 (3H, s) 2.28 (3H, s).

### Example 6:2

### 5-{5-[(4-Chlorophenyl)(methyl)amino]picolinoyl}-2-(2-phenylimidazol-1-yl)benzoic acid

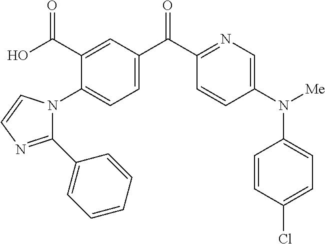

The title compound was prepared from 5-{5-[(4-chlorophenyl)methylamino]-pyridine-2-carbonyl}-2-fluoro-benzoic acid methyl ester (see Example 4:1, step (d)) and 2-phenylimidazole in accordance with Example 6:1.

¹H NMR (DMSO-d₆) δ: 13.3-13.0 (1H, br s) 8.48 (1H, d, J=1.8 Hz) 8.27-8.20 (2H, m) 8.02 (1H, d, J=8.8 Hz) 7.57-7.48 (3H, m) 7.42-7.35 (3H, m) 7.34-7.25 (6H, m) 7.16 (1H, d, J=1.2 Hz) 3.41 (3H, s).

### Example 7:1

### 5-{5-[(4-Chlorophenyl)(methyl)amino]picolinoyl}-2-{2-(3-chlorophenyl)pyrrol-1-yl}benzoic acid

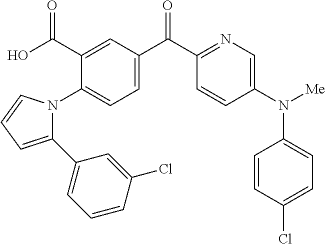

A mixture of 2-(3-chlorophenyl)pyrrole (80 mg, 0.45 mmol), 5-{5-[(4-chloro-phenyl)(methyl)amino]picolinoyl}-2-fluorobenzoic acid methyl ester (180 mg, 0.45 mmol, see Example 4:1, step (d)), 18-crown-6 (13.2 mg, 0.05 mmol), KF.Al₂O₃ (200 mg) was mixed in a vial using dry MeCN (3 mL) and then sealed and heated at 120° C. for 16 h. The reaction mixture was filtered through Celite and concentrated. Purification by chromatography and hydrolysis in accordance with Example 1, step (d) gave the title compound.

¹H NMR (DMSO-d₆) δ: 8.65 (1H, d, J=2.0 Hz) 8.25 (1H, dd, J=8.22.0 Hz) 8.17 (1H, d, J=3.0 Hz) 8.04 (1H, d, J=8.6 Hz) 7.42-7.37 (2H, m) 7.28 (1H, d, J=8.2 Hz) 7.21-6.99 (6H, m) 6.83-6.79 (2H, m) 6.42 (1H, dd, J=3.41.6 Hz) 6.36-6.34 (1H, m) 3.40 (3H, s).

### Example 7:2

### 5-{5-[(4-Chlorophenyl)(methyl)amino]picolinoyl}-2-(2-phenylpyrrol-1-yl)benzoic acid

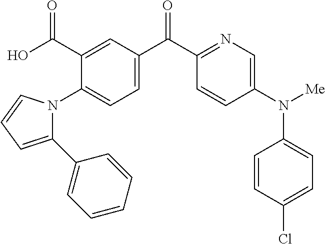

The title compound was prepared from 5-{5-[(4-chloro-phenyl)-methyl-amino]-pyridine-2-carbonyl}-2-fluoro-benzoic acid methyl ester (see Example 4:1, step (d)) and 2-phenylpyrrole in accordance with Example 7:1.

¹H NMR (DMSO-d₆) δ: 8.62 (1H, d, J=1.8 Hz) 8.23 (1H, dd, J=8.2, 1.8 Hz) 8.17 (1H, d, J=2.8 Hz) 8.03 (1H, d, J=9.0 Hz) 7.42-7.36 (2H, m) 7.29 (1H, d, J=8.2 Hz) 7.19-7.05 (8H, m) 6.84-6.79 (1H, m) 6.41 (1H, dd, J=3.4, 1.6 Hz) 6.37-6.34 (1H, m) 3.39 (3H, s).

### Examples 8

### 4-{5-[(4-Chlorophenyl)(methyl)amino]picolinoyl}[1,1′;2′,1″]terphenyl-2-carboxylic acid

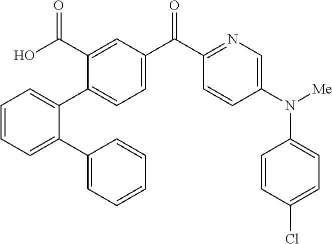

### (a) 2-Amino-5-{5-[(4-chlorophenyl)(methyl)amino]picolinoyl}benzoic acid methyl ester

THF (42 mL). Zn(s) (0.52 g, 8.0 mmol) and FeCl₃6H₂O (4.32 g, 16.0 mmol) was added to 2-azido-5-{5-[(4-chlorophenyl)(methyl)amino]picolinoyl}benzoic acid methyl ester (3.375 g, 8.0 mmol, see Example 4.1, step (e)) in EtOH (85 mL). The mixture was heated at rx for 30 min, Zn(s) (0.52 g, 8.0 mmol) was added and the mixture was heated at rx for 1 h. The mixture was filtered through Celite and concentrated. Extractive workup (EtOAc/THF/NaHCO₃ (aq)), drying (Na₂SO₄) and concentration gave the sub-title compound. Yield: 3.24 g (100%).

### (b) 5-{5-[(4-Chlorophenyl)(methyl)amino]picolinoyl}-2-iodobenzoic acid methyl ester

Water (32 mL) and HCl (aq, sat, 8.1 mL) was added to 2-amino-5-{5-[(4-chloro-phenyl)(methyl)amino]picolinoyl}benzoic acid methyl ester (3.24 g, 8.18 mmol) in MeCN (50 mL) at rt. The mixture was cooled to 0° C. and sodium nitrite (0.57 g, 8.31 mmol) in water (2.4 mL) was added. The mixture was stirred at 0° C. for 15 min and KI (1.38 g, 8.31 mmol) in water (2.4 mL) was added dropwise. The mixture was stirred at rt for 10 min and at rx for 15 min and concentrated. NaHCO₃ (aq, sat) was added. Extractive workup (EtOAc, brine), drying (Na₂SO₄) and purification by chromatography gave the sub-title compound. Yield: 2.6 g (64%).

### (c) 4-{5-[(4-Chloro-phenyl)methylamino]pyridine-2-carbonyl}-2′-trifluoro-methylbiphenyl-2-carboxylic acid

A mixture of 5-{5-[(4-chlorophenyl)(methyl)amino]picolinoyl}-2-iodobenzoic acid methyl ester (e.g. 0.40 mmol) and 2-phenylphenylboronic acid (e.g. 0.80 mmol) was employed and a coupling reaction was effected using an appropriate catalyst system and solvent (e.g. a palladium-based catalyst may be employed e.g. Pd(PPh₃)₄, optionally in the presence of another suitable additive) and an appropriate solvent system (e.g. toluene/water ratio 95/1). Reaction conditions such as those described herein may be deployed, and the product may be worked up and isolated in accordance with the procedures described herein.

12.9-12.7 (1H, br s) 8.34 (1H, d, J=1.6 Hz) 8.18 (1H, d, J=2.8 Hz) 7.98-7.90 (2H, m) 7.53-7.31 (7H, m) 7.25 (1H, dd, J=9.0, 3.0 Hz) 7.22-7.11 (6H, m) 7.06 (1H, d, J=8.2 Hz) 3.37 (3H, s)

### Example 9

### 5-{5-[(4-Chlorophenyl)(methyl)amino]picolinoyl}-2-(1-phenylimidazol-2-yl)benzoic acid

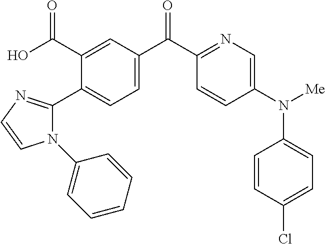

Butyl lithium (2.5 M in hexane, 0.20 mL, 0.505 mmol) was added dropwise to 1-phenylimidazole (72 mg, 0.50 mmol) in THF (1.5 mL) at −78° C. The mixture was stirred for 30 min at −78° C. and ZnCl₂ (0.5 M in THF, 3 mL, 1.05 mmol) was added. The temperature of the mixture was allowed to come to rt over 1.5 h and 5-{5-[(4-chlorophenyl)(methyl)amino]picolinoyl}-2-iodobenzoic acid methyl ester (152 mg, 0.3 mmol, see Example 8: 1, step (b)), and Pd(PPh₃)₄ (17 mg, 0.015 mmol) suspended in THF (1.5 mL) were added. The mixture was stirred at 75° C. for 4 h, diluted with EtOAc, washed with NH₄Cl, brine and water, dried (Na₂SO₄) and concentrated. Purification by chromatography and hydrolysis in accordance with Example 1, step (d), gave the title compound.

¹H NMR (DMSO-d₆) δ: 8.79 (1H, d, J=1.2 Hz) 8.15 (1H, d, J=2.8 Hz) 7.96 (1H, d, J=9.0 Hz) 7.88 (1H, dd, J=8.0, 1.6 Hz) 7.42-7.32 (6H, m) 7.28-7.21 (3H, m) 7.20-7.14 (2H, m) 7.09 (1H, dd, J=9.0, 3.0 Hz) 6.98 (1H, d, J=8.0 Hz) 3.38 (3H, s).

### Example 10

### (Z)-5-{5-[(4-Chlorophenyl)(methyl)amino]picolinoyl}-2-styrylbenzoic acid

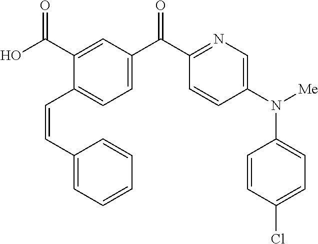

### (a) 5-{5-[(4-Chloro-phenyl)(methyl)amino]picolinoyl}-2-phenylethynylbenzoic acid

A mixture of 2-bromo-5-{5-[(4-chlorophenyl)(methyl)amino]picolinoyl}benzoic acid (230 mg, 0.5 mmol), phenylacetylene (153 mg, 1.5 mmol), Pd(PPh₃)₄ (53 mg, 0.05 mmol), BINAP (31 mg, 0.05 mmol), Cs₂CO₃ (244 mg, 0.75 mmol) and toluene (5 mL) was heated at 70° C. for 16 h. The mixture was allowed to cool and filtered through Celite. The solids were washed with EtOAc and the filtrates concentrated. Purification by chromatography gave the sub-title compound. Yield: 204 mg (84%).

### (b) (Z)-5-{5-[(4-chlorophenyl)(methyl)amino]picolinoyl}-2-styrylbenzoic acid

A mixture of 5-{5-[(4-chloro-phenyl)(methyl)amino]picolinoyl}-2-phenyl-ethynylbenzoic acid methyl ester (160 mg, 0.332 mmol), quinoline (4.3 mg, 0.033 mmol), Pd/BaSO₄ (5%) (34 mg), MeOH (4 mL) and EtOAc (4 mL) was stirred at rt for 4.5 h. The mixture was filtered through Celite, the solids washed with EtOAc and the filtrates concentrated. Purification by chromatography, hydrolysis in accordance with Example 1, step (d) and crystallization from EtOH gave the title compound. Yield: 60 mg (38%). ¹H-NMR (DMSO-d₆, 6) 13.5-12.8 (1H, br s) 8.55 (1H, d, J=1.2 Hz) 8.17 (1H, d, J=2.7 Hz) 8.00-7.86 (2H, m) 7.55-7.45 (2H, m) 7.38-7.31 (2H, m) 7.26 (1H, dd, J=9.0; 2.7 Hz) 7.23-7.10 (4H, m) 7.10-6.95 (3H, m) 6.68 (1H, d, J=12.1 Hz) 3.37 (3H, s).

### Example 11

Title compounds of the Examples were tested in the biological in vitro assay described above and were found to inhibit LTC₄ synthase. Thus, when the total concentration of title compounds in the assay was 10 μM, the following %-inhibition values where obtained.

| Ex. | % inhibition |
|---|---|
| 1 | 97 |
| 2:1 | 99 |
| 2:2 | 99 |
| 3:1 | 100 |
| 3:2 | 100 |
| 4:1 | 98 |
| 4:2 | 99 |
| 4:3 | 100 |

Title compounds of Examples 1 to 9 were also tested in the biological in vitro assay described above and were found to inhibit LTC₄ synthase. The IC₅₀ values are depicted below.

| Ex. | IC₅₀ (nM) |
|---|---|
| 1 | 288 |
| 2:1 | 326 |
| 2:2 | 214 |
| 3:1 | 154 |
| 3:2 | 36 |
| 4:1 | 116 |
| 4:2 | 47 |
| 4:3 | 45 |
| 4:4 | 16 |
| 4:5 | 56 |
| 4:6 | 107 |
| 5:1 | 297 |
| 5:2 | 104 |
| 5:3 | 177 |
| 5:4 | 65 |
| 5:5 | 116 |
| 5:6 | 115 |
| 5:7 | 250 |
| 6:1 | 235 |
| 6:2 | 486 |
| 7:1 | 163 |
| 7:2 | 64 |
| 8 | 244 |
| 9 | 217 |

1. A compound of formula I, 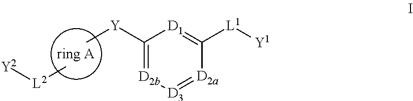
wherein
Y represents —C(O)— or —C(═N—OR²⁸)—;
R²⁸ represents hydrogen or C₁₋₆ alkyl optionally substituted by one or more fluoro atoms;
either one of D₂ₐ and D_{2b} represents D₂, and the other represents —C(T)═;
D₁, D₂ and D₃ respectively represent —C(R^{1a})═, —^{c}(R^{1b})═ and —C(R^{1c})═, or, each of these may alternatively and independently represent —N═;
ring A represents: 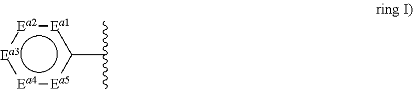
each of E^{a1}, E^{a2}, E^{a3}, E^{a4} and E^{a5} respectively represent —C(H)═, —C(R^{2b})═, —C(R^{2c})═, —C(R^{2d})═ and —C(H)═, or, each of E^{at} E^{a2}, E^{a3}, E^{a4} and E^{a5} may alternatively and independently represent —N═;
one of R^{2b}, R^{2c} and R^{2d} represents the requisite -L²-Y² group, and the others independently represent hydrogen, -L^{1a}-Y^{1a} or a substituent selected from X¹; 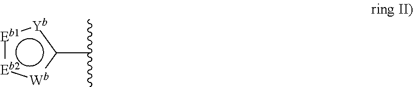
E^{b1} and E^{b2} respectively represent —C(R^{3a})═ and —C(R^{3b})═;
Y^{b} represents —C(R^{3c})═ or —N═;
W^{b} represents —N(R^{3d})—, —O— or —S—;
one of R^{3a}, R^{3b} and, if present, R^{3c} and R^{3d}, represents the requisite -L²-Y² group, and the remaining R^{3a}, R^{3b} and (if present) R^{3c} substituents represents hydrogen, -L^{1a}-Y^{1a} or a substituent selected from X², and the remaining R^{3d} substituent (if present) represents hydrogen or a substituent selected from R^{z1}; or 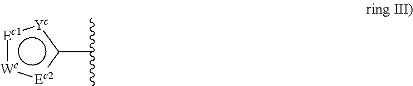
E^{c1} and E^{c2} respectively represent —C(R^{4a})═ and —C(R^{4b})═;
Y^{c} represents —C(R^{4c})═ or —N═;
W^{c} represents —N(R^{4d})—, —O— or —S—;
one of R^{4a}, R^{4b} and, if present, R^{4c} and R^{4d} represents the requisite -L²-Y² group, and the remaining R^{4a}, R^{4b} and (if present) R^{4c} substituents represent hydrogen, -L^{1a}-Y^{1a} or a substituent selected from X³, and the remaining R^{4d} substituent (if present) represents hydrogen or a substituent selected from R^{z2};
R^{z1} and R^{z2} independently represent a group selected from Z^{1a};
R^{1a}, R^{1b}, R^{1c}, independently represent hydrogen, a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
X¹, X² and X³ independently represent a group selected from Z^{2a}, or, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
Z^{1a} and Z^{2a} independently represent —R^{5a}, —C(O)R^{5b}, —C(O)OR^{5c}, —C(O)N(R^{6a})R^{7a}, —S(O)ₘR^{5j} or —S(O)₂N(R^{6h})R^{7h};
R^{5b} to R^{5h}, R^{5j}, R^{5k}, R⁵ⁿ, R^{6a} to R⁶ⁱ, R^{7a}, R^{7b}, R^{7d} and R^{7f} to R⁷ⁱ independently represent, on each occasion when used herein, H or R^{Sa}; or
any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from F, Cl, ═O, —OR^{5h} and/or R^{5a};
R⁵ⁱ, R^{5m} and R^{5p} independently represent R^{5a};
R^{5a} represents, on each occasion when used herein, C₁₋₆ alkyl optionally substituted by one or more substituents selected from halo, —CN, —N₃, ═O, —OR^{8a}, —N(R^{8b})R^{8c}, —S(O)ₙR^{8d}, —S(O)₂N(R^{8e})R^{8f} and/or —OS(O)₂N(R^{8g})R^{8h};
n represents 0, 1 or 2;
R^{8a}, R^{8b}, R^{8d}, R^{8e} and R^{8g} independently represent H or C₁₋₆ alkyl optionally substituted by one or more substituents selected from halo, ═O, —OR^{11a}, —N(R^{12a})R^{12b} and/or —S(O)₂-M¹;
R^{8c}, R^{8f} and R^{8h} independently represent H, —S(O)₂CH₃, —S(O)₂CF₃ or C₁₋₆ alkyl optionally substituted by one or more substituents selected from F, Cl, ═O, —OR^{13a}, —N(R^{14a})R^{14b} and/or —S(O)₂-M²; or
R^{8b} and R^{8c}, R^{8e} and R^{8f} or R^{8g} and R^{8h} may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from F, Cl, ═O and/or C₁₋₃ alkyl optionally substituted by one or more substituents selected from ═O and fluoro;
M¹ and M² independently represent —N(R^{15a})R^{15b} or C₁₋₃ alkyl optionally substituted by one or more fluoro atoms;
R^{11a} and R^{13a} independently represent H or C₁₋₃ alkyl optionally substituted by one or more fluoro atoms;
R^{12a}, R^{12b}, R^{14a}, R^{14b}, R^{15a} and R^{15b} independently represent H, —CH₃ or —CH₂CH₃,
T represents T¹, T², T³, T⁴, T⁵, T⁶ or T⁷;
T¹ is an aromatic ring represented by the following substructure: 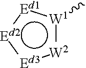
in which:
(i) W¹ represents —C═;
W² represents —C(R¹)═ or —C(Y³)═; and
one of E^{d1}, E^{d2} or E^{d3} represents —N(R²)—, —N(Y⁴)—, —O— or —S—, and the other two independently represent —C(R¹)═, —C(Y³)═ or —N═,
wherein there is one Y³ or Y⁴ group present;
(ii) W¹ represents —C═;
W² represents —N(Y⁴)—;
E^{d1}, E^{d2} and E^{d3} independently represent —C(R¹)═ or —N═; or
iii) W¹ represents —N—;
W² represents —C(R¹)═ or —C(Y³)═;
E^{d1}, E^{d2} and E^{d3} independently represent —C(R¹)═, —C(Y³)═ or —N═,
wherein there is one Y³ group present;
T² is an aromatic ring represented by the following substructure: 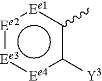
in which:
one of E^{e1}, E^{e2} E^{e3} or E^{e4} represents —C(R¹)═ and the others independently represent —C(R¹)═ or —N═;
T³ is an aromatic bicyclic ring represented by the following substructure: 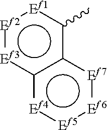
any three of E^{f1} to E^{f7} represent —C(R¹)═, and the others independently represent —C(R¹)═ or —N═;
T⁴ is an aromatic bicyclic ring represented by the following substructure: 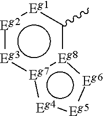
E^{g1}, E^{g2}, E^{g3} and E^{g5} independently represent —C(R¹)═ or —N═;
one of E^{g4} and E^{g6} represents —N(R²)—, —O— or —S— and the other represents —C(R¹)═ or —N═;
E^{g7} and E^{g8} both represent a carbon atom, or one of E^{g7} and E^{g8} represents a carbon atom and the other represents a nitrogen atom,
but wherein in the bicyclic ring, no more than four nitrogen atoms are present (i.e. no more than four of E^{g1} to E^{g8} may represent a nitrogen atom represented by —N═ or —N(R²)—);
T⁵ is an aromatic bicyclic ring represented by the following substructure: 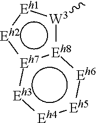
E^{h3}, E^{h4}, E^{h5} and E^{h6} independently represent —C(R¹)═ or —N═;
(i) W³ represents —C═;
E^{h1} represents —C(R¹)═ or —N═;
E^{h2} represents —N(R²)—, —O— or —S—, or, provided that E^{h7} or E^{h8} is a nitrogen atom, —C(R¹)═;
E^{h7} and E^{h8} both represent a carbon atom, or one of E^{h7} and E^{h8} represents a carbon atom and the other represents a nitrogen atom;
(ii) W³ represents —N—;
E^{h1} and E^{h2} independently represent —C(R¹)═ or —N═;
E^{h1} and E^{h8} both represent a carbon atom,
but wherein in the bicyclic ring, no more than four nitrogen atoms are present (i.e. no more than four of W³, and E^{h1} to E^{h8} may represent a nitrogen atom represented by —N═ or —N(R²)—);
T⁶ is a ring represented by the following substructure: 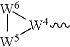
W⁴ represents —C(R³)— or —N—;
when W⁴ represents —C(R³)—, then W⁵ represents —C(R³)(Y⁵)— or —N(Y⁶)—;
when W⁴ represents —N—, then W⁵ represents —C(R³)(Y⁵)—;
W⁶ represents an C₁₋₅ alkylene or C₁₋₅ heteroalkylene chain both of which are optionally substituted by one or more substituents selected from G^{x};
T⁷ is a cyclic or acyclic alkene represented by the following substructure: 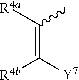
R^{4a} and R^{4b} independently represent G^{w}, or R^{4a} and R^{4a} are connected together to form, along with the two alkene carbons to which they are necessarily attached, a C₃₋₇ cycloalkylene or 3- to 8-membered heterocycloalkylene ring both optionally substituted by one or more substituents selected from G^{x};
each R¹ represents, on each occasion when used herein, hydrogen, halo, —R^{25a}, —C(O)R^{25b}—CN, —C(O)N(R^{26a})R^{27a}, —N(R^{26b})R^{27b}, —N(R^{25c})C(O)R^{26c}, —N(R^{25d})C(O)OR^{26d}, —OR^{25e}, —OS(O)₂R^{25f}, —S(O)ₘ₁R^{25g}, —OC(O)R^{25h} or —S(O)₂N(R^{26e})R^{27e};
each R² represents, on each occasion when used herein, hydrogen, —R^{25a}, —C(O)R^{25b} or —C(O)N(R^{26a})R^{27a};
each R³ represents, on each occasion when used herein, hydrogen, —R^{35a}, —CN, —N(R^{36b})R^{37b} or —OR^{35d};
G^{w} represents hydrogen, halo, —R^{45a}, —C(O)R^{45b}, —CN, —C(O)N(R^{46a})R^{47a}, —N(R^{46b})R^{47b}, —N(R^{45c})C(O)R^{46c}, —N(R^{45d})C(O)OR^{46d}, —OR^{45e}, —OS(O)₂R^{45f}, —S(O)ₘ₁R^{45g}, —OC(O)R^{45h} or —S(O)₂N(R^{46e})R^{47e};
G^{x} represents F, —R^{55a}, —C(O)R^{55b}, —CN, —C(O)N(R^{56a})R^{57a}, —N(R^{56b})R^{57b}, —N(R^{55c})C(O)R^{56c}, —N(R^{55d})C(O)OR^{56d}, —OR^{55e}, —OS(O)₂R^{55f}, —S(O)ₘ₁R^{55g}, —OC(O)R⁵⁵″, —S(O)₂N(R^{56e})R^{57e} or ═O;
m1 represents, on each occasion when used herein, 0, 1 or 2;
R^{25b} to R^{25e}, R^{25g}, R^{25h}, R^{26a} to R^{26e}, R^{27a}, R^{27b} and R^{27e} independently represent, on each occasion when used herein, H or R^{25a}; or
any of the pairs R^{26a} and R^{27a}, R^{26b} and R^{27b}, R^{26e} and R^{27e}, may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from fluoro, ═O, —O—C₁₋₄ alkyl and/or C₁₋₄ alkyl;
R^{25f} represents R^{25a};
R^{36b}, R^{37b} and R^{35d} independently represent hydrogen or R^{35a}; or
R^{36b} and R^{37b} may be linked together to form a 3- to 6-membered ring optionally containing one further heteroatom, and which ring is optionally substituted by one or more substituents selected from F and methyl;
R^{45b}, R^{46a}, R^{47a}, R^{46c}, R^{46d}, R^{45h}, R^{46e} and R^{47e} independently represent hydrogen or R^{45a};
R^{45g}, R^{46b}, R^{47b}, R^{45c}, R^{45d}, R^{45e} and R^{45f} independently represent R^{45a}; or
any of the pairs R^{46a} and R^{47a}, R^{46b} and R^{47b}, and R^{46e} and R^{47e} may be linked together to form a 3- to 6-membered ring optionally containing one further heteroatom, and which ring is optionally substituted by one or more substituents selected from F and methyl;
R^{55b} to R^{55e}, R^{55g}, R^{55h}, R^{56a} to R^{56e}, R^{57a}, R^{57b} and R^{57e} independently represent, on each occasion when used herein, H or R^{55a}; or
any of the pairs R^{56e} and R^{57a}, R^{56b} and R^{57b}, R^{56e} and R^{57e}, may be linked together to form a 3- to 6-membered ring optionally containing one further heteroatom, and which ring is optionally substituted by one or more substituents selected from F and methyl;
R^{25a} represents, on each occasion when used herein, C₁₋₆ alkyl optionally substituted by one or more substituents selected from fluoro, —CN, ═O and —O—C₁₋₄ alkyl;
R^{35a}, R^{45a} and R^{55a} independently represent C₁₋₄ alkyl optionally substituted by one or more fluoro atoms;
Y², Y³, Y⁴, Y⁵, Y⁶ and Y⁷ independently represent, on each occasion when used herein:
(a) an aryl group or a heteroaryl group (both of which groups are optionally substituted by one or more substituents selected from A); or
(b) C₁₋₁₂ alkyl or a heterocycloalkyl group, both of which are optionally substituted by one or more substituents selected from G¹ and/or Z¹,
but wherein at least one Y², Y³, Y⁴, Y⁵, Y⁶, Y⁷ group is present that represents an aryl group or a heteroaryl group (both of which groups are optionally substituted by one or more substituents selected from A);
A represents, on each occasion when used herein:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents, on each occasion when used herein, halo, cyano, —N₃, —NO₂, —ONO₂ or -A¹-R^{16a};
wherein A¹ represents a single bond or a spacer group selected from —C(O)A²-, —S—, —S(O)ₘₓA³-, —N(R^{17a})A⁴- or —OA⁵-, in which:
A² represents a single bond, —O—, —N(R^{17b})— or —C(O)—;
A³ represents a single bond, —O— or —N(R^{17c})—;
A⁴ and A⁵ independently represent a single bond, —C(O)—, —C(O)N(R^{17d})—, —C(O)O—, —S(O)₂— or —S(O)₂N(R^{17e})—;
Z¹ represents, on each occasion when used herein, ═O, ═S, ═NOR^{16b}, ═NS(O)₂N(R^{17f})R^{16c}, ═NCN or ═C(H)NO₂;
B represents, on each occasion when used herein:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents, on each occasion when used herein, halo, cyano, —N₃, —NO₂, —ONO₂ or -A⁶-R^{18a};
wherein A⁶ represents a single bond or a spacer group selected from —C(O)A⁷-, —S—, —N(R^{19a})A⁹- or —OA¹⁰-, in which:
A⁷ represents a single bond, —O—, —N(R^{19b})— or —C(O)—;
A⁸ represents a single bond, —O— or —N(R^{19c})—;
A⁹ and A¹⁰ independently represent a single bond, —C(O)—, —C(O)N(R^{19d})—, —C(O)O—, —S(O)₂— or —S(O)₂N(R^{19e})—;
Z² represents, on each occasion when used herein, ═O, ═S, ═NOR^{18b}, ═NS(O)₂N(R^{19f})R^{18c}, ═NCN or ═C(H)NO₂;
R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by one or more substituents selected from G³ and/or Z³; or
any pair of R^{16a} to R^{16c} and R^{17a} to R^{17f}, and/or R^{18a} to R^{18c} and R^{19a} to R^{19f}, may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents, on each occasion when used herein, halo, cyano, —N₃, —NO₂, —ONO₂ or -A¹¹-R^{20a};
wherein A¹¹ represents a single bond or a spacer group selected from —C(O)A¹²-, —S—, —S(O)ₘₓA¹³-, —N(R^{21a})A¹⁴- or —OA¹⁶-, in which:
A¹² represents a single bond, —O—, —N(R^{2lb})— or —C(O)—;
A¹³ represents a single bond, —O— or —N(R^{21c})—;
A¹⁴ and A¹⁵ independently represent a single bond, —C(O)—, —C(O)N(R^{21d})—, —C(O)O—, —S(O)₂— or —S(O)₂N(R^{21e})—;
Z³ represents, on each occasion when used herein, ═O, ═S, ═NOR^{20b}, ═NS(O)₂N(R^{21f})R^{20c}, ═NCN or ═C(H)NO₂;
R^{20a}, R^{20b}, R^{20c}, R^{21a}, R^{21b}, R^{21c}, R^{21d}, R^{21e} and R^{21f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{22a})R^{23a}, —OR^{22b} and ═O; and
iii) an aryl or heteroaryl group, both of which are optionally substituted by one or more substituents selected from halo, C₁₋₄ alkyl (optionally substituted by one or more substituents selected from ═O, fluoro and chloro), —N(R^{22c})R^{23b} and —OR^{22d}; or
any pair of R^{20a} to R^{20c} and R^{21a} to R^{21f} may, for example when present on the same or on adjacent atoms, be linked together to form with those, or other relevant, atoms a further 3- to 8-membered ring, optionally containing 1 to 3 heteroatoms and/or 1 or 2 double bonds, which ring) is optionally substituted by one or more substituents selected from halo, C₁₋₄ alkyl, —N(R^{22e})R^{23c}, —OR^{22f} and ═O;
Y¹ and Y^{1a} independently represent —C(O)OR^{9a} or 5-tetrazolyl;
R^{9a} represents hydrogen or C₁₋₄ alkyl optionally substituted by one or more halo atoms;
and L^{1a} independently represent a single bond or —(CH₂)ₚ-Q-(CH₂)_{q}—;
Q represents —C(R^{y1})(R^{y2})—, —C(O)—, —N(R^{y3})— or —O—;
p and q independently represent 0, 1 or 2, but wherein the sum of p and q does not exceed 2;
L² represents a single bond or a spacer group selected from —C(R^{y4})(R^{y5})—, —N(R^{17a})-A¹⁶-, and —OA¹⁷-;
A¹⁶ represents a direct bond, —C(O)—, —C(O)N(R^{17b})—, —C(O)C(R^{y6})(R^{y7})— or —S(O)₂—;
A¹⁷ represents a direct bond or —C(R^{y8})(R^{y9})—;
R^{y1}, R^{y2}, R^{y4}, R^{y5}, R^{y6}, R^{y7}, R^{y8} and R^{y9} independently represent H, fluoro or C₁₋₃ alkyl optionally substituted by one or more fluoro atoms; or
R^{y1} and R^{y2}, R^{y4} and R^{y5}, R^{y6} and R^{y7} and R^{y8} and R^{y9} may be linked together to form a 3- to 6-membered ring optionally substituted by one or more substituents selected from fluoro and C₁₋₂ alkyl;
R^{y3} represents hydrogen or C₁₋₃ alkyl;
R^{17a} and R^{17b} independently represent hydrogen, C₁₋₆ alkyl (optionally substituted by one or more substituents selected from fluoro, —CN, —OH, —OCH₃, —OCH₂CH₃ and/or ═O), aryl or heteroaryl (both of which latter two groups are optionally substituted by one or more substituents selected from halo, —R^{18a}, —C(O)R^{18b}, —CN, —C(O)N(R^{18c})R^{18d}, —N(R^{18e})R^{18f}, —N(R^{18g})C(O)R^{18h}, —N(R¹⁸ⁱ)C(O)OR^{18j}, —OR^{18k}, —OS(O)₂R^{18m}, —S(O)ₘR¹⁸ⁿ, —OC(O)R^{18p} or —S(O)₂N(R^{18q})R^{18r});
m represents, on each occasion when used herein, 0, 1 or 2;
mx represents, on each occasion when used herein, 1 or 2;
R^{18a}, R^{18b}, R^{18c}, R^{18d}, R^{18e}, R^{18f}, R^{18g}, R^{18h}, R¹⁸ⁱ, R^{18j}, R^{18k}, R¹⁸ⁿ, R^{18p}, R^{18q} and R^{18r} independently represent hydrogen or C₁₋₃ alkyl optionally substituted by one or more fluoro atoms;
R^{18m} represents C₁₋₃ alkyl optionally substituted by one or more fluoro atoms;
or a pharmaceutically-acceptable salt thereof.

2. A compound as claimed in claim 1, wherein the compounds of formula I represents: 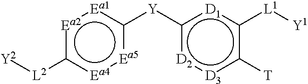
in which:
Y represents —C(O)— or —C(═N—OR²⁸)—;
R²⁸ represents hydrogen or C₁₋₄ (e.g. C₁₋₂) alkyl;
D₁, D₂ and D₃ respectively represent —C(R^{1a})═, —C(R^{1b})═ and —C(R^{1c})═;
R^{1a}, R^{1b} and R^{1c} independently represent hydrogen, R^{5a}, halo or —CN;
each of E^{a1}, E^{a2}, E^{a4} and E^{a5} respectively represent —C(H)═, —C(R^{2b})═, —C(R^{2d})═ and —C(H)═, or, one or two of E^{a1}, E^{a2}, E^{a4} and E^{a5} may alternatively and independently represent —N═;
R^{2b} and R^{2d} independently represent hydrogen or a substituent selected from X¹;
X¹ represents a group selected from R^{5a}, halo or —CN;
R^{5a} represents, on each occasion when used herein, C₁₋₆ alkyl optionally substituted by one or more substituents selected from ═O, halo, —CN and —N₃;
Y¹ represents —C(O)OR^{9a};
T represents T¹, T², T⁵ or T⁷, i.e.: 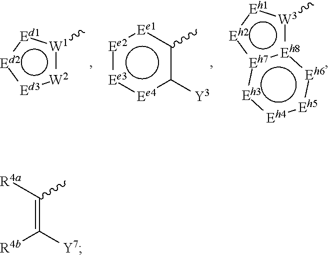
W¹ represents —N— or —C═;
E^{d1} represents —N═ or —C(R¹)═;
E^{d2} represents —N═, —C(R¹)═ or —C(Y³)═;
E^{d3} represents —N═, —C(Y³)═ or —C(R¹)═;
W² represents —C(Y³)═ or —C(R¹)═ or, when W¹ represents —C—, then W² may represent —N(Y⁴)═;
E^{e1}, E^{e2}, E^{e3} and E^{e4} independently represent —C(R¹)═(in which each R¹ is preferably hydrogen);
W³ represents —N—;
E^{h2} represents —N—;
E^{h1} represents —C(R¹)═;
E^{h3}, E^{h4}, H^{h5} and E^{h6} independently represent —C(R¹)═;
R^{4a} and R^{4b} independently represent hydrogen;
Y², Y³, Y⁴ and Y⁷ independently represent aryl or pyridyl, both of which are optionally substituted by one or more substituents selected from A;
each R¹ independently represents hydrogen, halo, —CN or R^{25a};
R^{25a} represents C₁₋₆ alkyl optionally substituted by one or more fluoro substituents;
A represents G¹ or C₁₋₆ alkyl optionally substituted by one or more substituents selected from G¹;
G¹ represents halo;
R^{9a} represents hydrogen or C₁₋₆ alkyl;
L¹ independently represent a single bond;
L² represents —N(R^{17a})-A¹⁶- or —OA¹⁷-;
A¹⁶ and A¹⁷ independently represent a direct bond;
R^{17a} represents hydrogen or C₁₋₆ alkyl.

3. A compound as claimed in claim 1, wherein:
T¹ represents (when W¹ represents —N—): 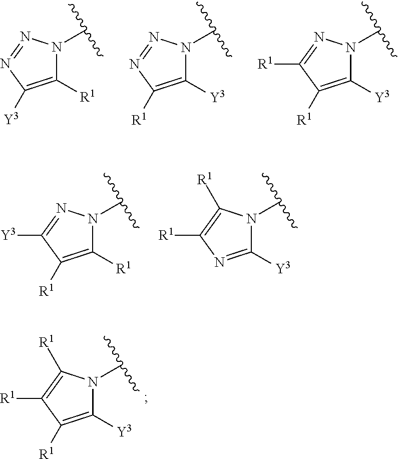
T¹ represents (when W¹ represents —C═): 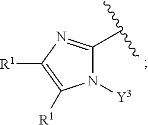
T² represents: 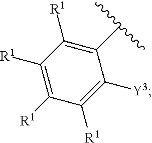
T⁵ represents: 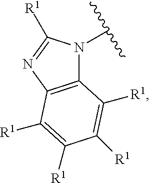
in which each R¹ is independent and as defined in claim 1 or claim 2, and Y³ is as defined in claim 1 or claim 2.

4. A compound as claimed in claim 1, wherein:
R^{1a}, R^{1b} and R^{1c} independently represent hydrogen;
ring A represents ring (I) (as depicted in claim 2);
E^{a1} and E^{a5} independently represent —C(H)═ or either one of these represents —N═;
E^{a2}, E^{a3} and E^{a4} respectively represent —C(R^{2b})═, —C(R^{2c})═ and —C(R^{2d})═;
R^{2b} represents H;
R^{2c} represents the requisite -L²-Y² group (as depicted in claim 2);
R^{2d} represents H;
T represents T¹ or T⁵;
W¹ represents —N—;
E^{d1} represents —C(R¹)═ or, preferably, —N═;
E^{d2} represents —N═;
E^{d3} represents —C(R¹)═ or —C(Y³)═;
W² represents —C(R¹)═ or —C(Y³)═;
one of E^{d3} and W² represents —C(R¹)═ and the other represents —C(Y³)═;
Y² and Y³, Y⁴, Y⁵, Y⁶ or Y⁷ both represent aryl or heteroaryl optionally substituted as defined in
any one of the preceding claims;
W³ represents —N—;
E^{h2} represents —N═;
E^{h1}, E^{h3}, E^{h4}, E^{h5}, E^{h6}, E^{h7} and independently represent —C(R¹)═;
R¹ represents hydrogen or R^{25a};
R^{25a} represents C₁₋₃ alkyl;
there are no -L^{1a}-Y^{1a} groups present;
R^{17a} represents hydrogen or C₁₋₆ alkyl optionally substituted by one or more substituents selected from —OCH₃ and —CN;
G¹ represents halo or -A¹-R^{16a};
A¹ represents a single bond or —OA⁵-;
A⁵ represents a single bond;
R^{16a} represents hydrogen or C₁₋₆ alkyl optionally substituted by one or more substituents selected from G³; and/or
G³ represents halo.

5. A compound as claimed in claim 1, wherein when there is a Y³, Y⁴, Y⁵, Y⁶ or Y⁷ group present, then it represents: a cycloalkyl or heterocycloalkyl group (both of which are optionally substituted by one or more substituents selected from G¹ and/or Z¹) or aryl or heteroaryl (both of which are optionally substituted by one or more substituents selected from A).

6. A compound as claimed in claim 1, wherein both Y² and, if present, Y³ to Y⁷ represent aryl or heteroaryl (both of which are optionally substituted by one or more substituents selected from A).

7. A compound of formula I as defined in claim 1, or a pharmaceutically acceptable salt thereof, for use as a pharmaceutical.

8. A pharmaceutical formulation including a compound of formula I, as defined in claim 1, or a pharmaceutically acceptable salt thereof, in admixture with a pharmaceutically acceptable adjuvant, diluent or carrier.

9. A compound, as defined in claim 1, or a pharmaceutically acceptable salt thereof, for use in the treatment of a disease in which inhibition of the synthesis of leukotriene C₄ is desired and/or required.

10. (canceled)

11. A compound as claimed in claim 9, wherein the disease is a respiratory disease, inflammation and/or has an inflammatory component.

12. A compound as claimed in claim 11 wherein the disease is an allergic disorder, asthma, childhood wheezing, a chronic obstructive pulmonary disease, bronchopulmonary dysplasia, cystic fibrosis, an interstitial lung disease, an ear nose and throat disease, an eye disease, a skin diseases, a rheumatic disease, vasculitis, a cardiovascular disease, a gastrointestinal disease, a urologic disease, a disease of the central nervous system, an endocrine disease, urticaria, anaphylaxis, angioedema, oedema in Kwashiorkor, dysmenorrhea, a burn-induced oxidative injury, multiple trauma, pain, toxic oil syndrome, endotoxin chock, sepsis, a bacterial infection, a fungal infection, a viral infection, sickle cell anaemia, hypereosinofilic syndrome, or a malignancy.

13. A compound as claimed in claim 12, wherein the disease is an allergic disorder, asthma, rhinitis, conjunctivitis, COPD, cystic fibrosis, dermatitis, urticaria, an eosinophilic gastrointestinal disease, an inflammatory bowel disease, rheumatoid arthritis, osteoarthritis or pain.

14. A method of treatment of a disease in which inhibition of the synthesis of leukotriene C₄ is desired and/or required, which method comprises administration of a therapeutically effective amount of a compound of formula I as defined in claim 1, or a pharmaceutically-acceptable salt thereof, to a patient suffering from, or susceptible to, such a condition.

15. A combination product comprising:
(A) a compound of formula I as defined in claim 1, or a pharmaceutically-acceptable salt thereof; and
(B) another therapeutic agent that is useful in the treatment of a respiratory disorder and/or inflammation,
wherein each of components (A) and (B) is formulated in admixture with a pharmaceutically-acceptable adjuvant, diluent or carrier.

16. A combination product as claimed in claim 15 which comprises a pharmaceutical formulation including a compound of formula I as defined in claim 1, or a pharmaceutically-acceptable salt thereof, another therapeutic agent that is useful in the treatment of a respiratory disorder and/or inflammation, and a pharmaceutically-acceptable adjuvant, diluent or carrier.

17. A combination product as claimed in claim 15 which comprises a kit of parts comprising components:
(a) a pharmaceutical formulation including a compound of formula I as defined in claim 1, or a pharmaceutically-acceptable salt thereof, in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier,
which components (a) and (b) are each provided in a form that is suitable for administration in conjunction with the other.

18. A process for the preparation of a compound of formula I as defined in claim 1, which process comprises:
(i) for compounds of formula I in which Y represents —C(O)—, oxidation of a compound of formula II, 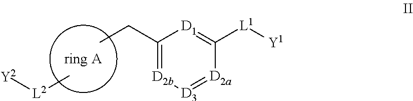
wherein ring A, D₁, D₂ₐ, D_{2b}, D₃, L¹, Y¹, L² and Y² are as defined in claim 1;
(ii) for compounds of formula I in which L² represents —N(R^{17a})A¹⁶- in which R^{17a} represents H, reaction of a compound of formula III, 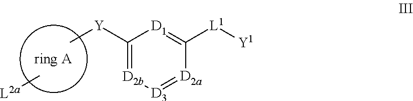
or a protected derivative thereof wherein L^{2a} represents —NH₂, and Y, ring A, D₁, D₂ₐ, D_{2b}, D₃, L¹ and Y¹ are as defined in claim 1 with:
(A) when A¹⁶ represents —C(O)N(R^{17b})—, in which R^{17b} represents H:
(a) a compound of formula IV,
Y^{a}—N═C═O IV;
or
(b) with CO (or a reagent that is a suitable source of CO) or a reagent such as phosgene or triphosgene in the presence of a compound of formula V,
Y²—NH₂ V
wherein, Y² is as defined in claim 1;
(B) when A¹⁶ represents a single bond, with a compound of formula VI,
Y²-L^{a} VI
wherein L^{a} represents a suitable leaving group and Y² is as defined in claim 1;
(C) when A¹⁶ represents —S(O)₂—, —C(O)— or —C(O)—C(R^{y6})(R^{y7})—, with a compound of formula VII,
Y²-A^{16a}-L^{a} VII
wherein A^{16a} represents —S(O)₂—, —C(O)— or —C(O)—C(R^{y6})(R^{y7})—, and Y² and L^{a} are as defined above or in claim 1;
(iii) for compounds of formula I in which one of L² represents —N(R^{17a})C(O)N(R^{17b})—, in which R^{17a} and R^{17b} both represent H, and, preferably, Y is —C(O)— or R²⁸ is C₁₋₆ alkyl optionally substituted by one or more halo atoms, reaction of a compound of formula VIII, 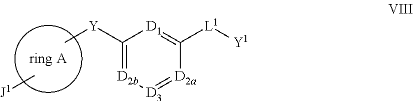
wherein J¹ represents —N═C═O, and Y, ring A, D₁, D₂ₐ, D_{2b}, D₃, L¹ and Y¹ are as defined in claim 1, with a compound of formula V as defined above;
(iv) for compounds of formula I in which, preferably, Y is —C(O)— or R²⁸ is C₁₋₆ alkyl optionally substituted by one or more halo atoms, reaction of a compound of formula IX, 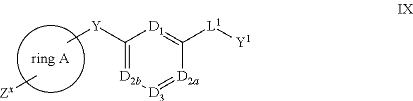
wherein Z^{x} represents a suitable leaving group, and Y, ring A, D₁, D₂ₐ, D_{2b}, D₃, L¹ and Y¹ are as defined in claim 1, with a compound of formula X,
Y²-L²-H X
wherein L² and Y² is as defined in claim 1 or above;
(iva) for compounds of formula I in which, preferably, Y is —C(O)— or R²⁸ is C₁₋₆ alkyl optionally substituted by one or more halo atoms, reaction of a compound of formula IXA, 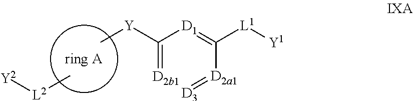
wherein one of D^{2b1} and D^{2a1} represents —C(—Z^{y})═, and the other represents D₂, Z^{y} represents (independently) a group defined in respect of Z^{x} above or, alternatively, Z^{y} may represent —N₃, and Y, ring A, D₁, D₂ₐ, D_{2b}, D₃, L¹, Y¹, C and Y² are as defined in claim 1, with a compound of formula XA,
T-H XA
wherein T is as defined in claim 1 or, in the case of reaction with a compound of formula IXA in which Z^{y} is —N₃ (and the compound to be formed is one in which T represents a triazolyl group), with an appropriate alkynyl compound;
(v) compounds of formula I in which there is a R^{17a} or R^{17b} group present that does not represent hydrogen (or if there is R⁵, R⁶, R⁷, R⁸, R⁹, R¹¹, R¹², R¹³, R¹⁴, R¹⁶, R¹⁷ or R¹⁸ group present, which is attached to a heteroatom such as nitrogen or oxygen, and which does/do not represent hydrogen), may be prepared by reaction of a corresponding compound of formula I in which such a group is present that does represent hydrogen with a compound of formula XI,
R^{wy}-L^{b} XI
wherein R^{wy} represents either R^{17a} or R^{17b} (as appropriate) as hereinbefore in claim 1 provided that it does not represent hydrogen (or R^{wy} represents a R⁵ to R¹⁸ group in which those groups do not represent hydrogen), and L^{b} represents a suitable leaving group;
(vi) for compounds of formula I that contain only saturated alkyl groups, reduction of a corresponding compound of formula I that contains an unsaturation;
(vii) for compounds of formula I in which Y¹ and/or, if present, Y^{1a} represents —C(O)OR^{9a}, in which R^{9a} represent hydrogen, hydrolysis of a corresponding compound of formula I in which R^{9a} does not represent H;
(viii) for compounds of formula I in which Y¹ and/or, if present, Y^{1a} represents —C(O)OR^{9a} and R^{9a} does not represent H:
(A) esterification (or the like) of a corresponding compound of formula I in which R^{9a} represents H; or
(B) trans-esterification (or the like) of a corresponding compound of formula I in which
R^{9a} does not represent H (and does not represent the same value of the corresponding
R^{9a} group in the compound of formula Ito be prepared),
in the presence of the appropriate alcohol of formula XII,
R^{9za}OH XII
in which R^{9za} represents R^{a} provided that it does not represent H;
(ix) for compounds of formula I in which Y¹ and/or, if present, Y^{1a} represents —C(O)OR^{9a}, in which R^{9a} is other than H, and L¹ is as hereinbefore defined, provided that it does not represent —(CH₂)ₚ-Q-(CH₂)_{q}— in which p represents 0 and Q represents —O—, reaction of a compound of formula XIII, 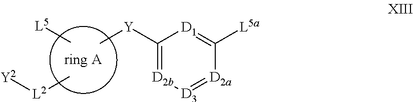
wherein at least one of L⁵ and L^{5a} represents an appropriate alkali metal group, a —Mg-halide, a zinc-based group or a suitable leaving group such as halo or —B(OH)₂, or a protected derivative thereof, and the other may represent -L¹-Y¹ or -L^{1a}-Y^{1a} (as appropriate), and Y, ring A, D₁, D₂ₐ, D_{2b}, D₃, L² and Y² are as defined in claim 1, with a compound of formula XIV,
L⁶-L^{xy}-Y^{b} XIV
wherein L^{xy} represents L¹ or L^{1a} as hereinbefore defined (provided that it does not represent —(CH₂)ₚ-Q-(CH₂)_{q}— in which p represents 0 and Q represents —O—) and Y^{b} represents —C(O)OR^{9a}, in which R^{9a} is other than H, and L⁶ represents a suitable leaving group;
(x) compounds of formula I in which L¹ preferably represents a single bond, and Y¹ represents 5-tetrazolyl, or, L^{1a} and Y^{1a}, if present, represent those groups defined by L¹ and Y¹, may be prepared in accordance with the procedures described in international patent application WO 2006/077366;
(xi) for compounds of formula I in which L¹ and/or, if present, L^{1a} represent a single bond, and Y¹ and/or, if present, Y^{1a} represent —C(O)OR^{9a} in which R^{9a} is H, reaction of a compound of formula XIII as hereinbefore defined but in which L^{6a} represents either:
(I) an alkali metal; or
(II) —Mg-halide,
with carbon dioxide, followed by acidification;
(xii) for compounds of formula I in which L¹ and/or, if present, L^{1a} represent a single bond, and Y¹ and/or, if present, Y^{1a} represent —C(O)OR^{9a}, reaction of a corresponding compound of formula XIII as hereinbefore defined but in which L^{6a} is a suitable leaving group with CO (or a reagent that is a suitable source of CO), in the presence of a compound of formula XV,
R^{9a}OH XV
wherein R^{9a} is as defined above;
(xiii) for compounds of formula I in which Y represents —C(O)—, reaction of either a compound of formula XVI or XVII, 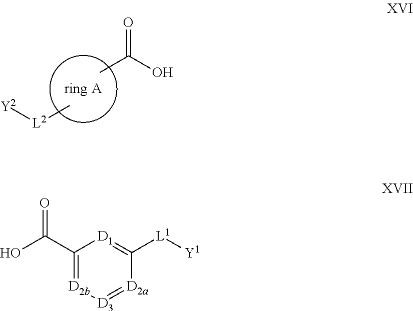
respectively with a compound of formula XVIII or XIX, 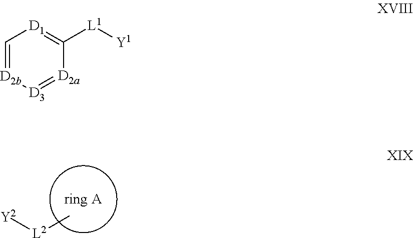
wherein (in all cases) ring A, D₁, D₂ₐ, D_{2b}, D₃, L¹, Y¹, L² and Y² are as defined in claim 1;
(xiv) for compounds of formula I in which Y represents —C(O)—, reaction of either a compound of formula XX or XXI, 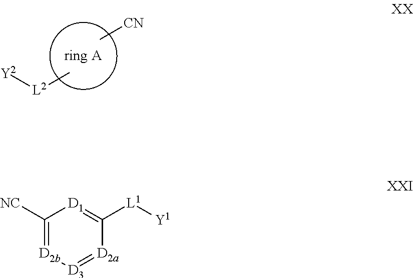
with a compound of formula XXII or XXIII, 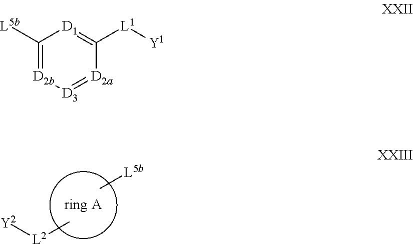
respectively, wherein L^{5b} represents L⁵ as hereinbefore defined provided that it does not represent -L¹-Y¹, an alkali metal or a —Mg-halide, and (in all cases) ring A, D₁, D₂ₐ, D_{2b}, D₃, L¹, Y¹, L² and Y² are as defined in claim 1;
(xv) for compounds of formula I in which Y represents —C(O)—, reaction of an activated derivative of a compound of formula XVI or XVII as defined above, with a compound of formula XXII or XXIII (as defined above), respectively;
(xvi) for compounds of formula I in which Y represents —C(═N—OR²⁸)—, reaction of a corresponding compound of formula I, with a compound of formula XXIIIA,
H₂N—O—R²⁸ XXIIIA
wherein R²⁸ is represents hydrogen or C₁₋₆ alkyl optionally substituted by one or more halo atoms;
(xvii) for compounds of formula I in which Y represents —C(═N—OR²⁸)— and R²⁸ represents C₁₋₆ alkyl optionally substituted by one or more halo atoms, reaction of a corresponding compound of formula I, in which R²⁸ represents hydrogen, with a compound of formula XXIIIB,
R^{28a}-L⁷ XXIIIB
wherein R^{28a} represents R²⁸, provided that it does not represent hydrogen and L⁷ represents a suitable leaving group.

19. A process for the preparation of a pharmaceutical formulation as defined in claim 8, which process comprises bringing into association a compound of formula I, as defined in claim 1, or a pharmaceutically acceptable salt thereof with a pharmaceutically-acceptable adjuvant, diluent or carrier.

20. A process for the preparation of a combination product as defined in claim 15, which process comprises bringing into association a compound of formula I, as defined in claim 1, or a pharmaceutically acceptable salt thereof with the other therapeutic agent that is useful in the treatment of a respiratory disorder and/or inflammation, and at least one pharmaceutically-acceptable adjuvant, diluent or carrier.
